SEQUENCE LISTING (66 sequences)

SEQ ID NO: 1 - protein, 628 aa, Escherichia coli
MSFSEFYQRSINEPEQFWAEQARRIDWQTPFTQTLDHSNPPFARWFCEGRTNLCHNAIDRWLEKQPEALALIAVSSETEEERTFTFRQLHDEVNAVASMLRSLGVQRGDRVLVYMPMIAEAHITLLACARIGAIHSVVFGGFASHSVAARIDDAKPVLIVSADAGARGGKIIPYKKLLDDAISQAQHQPRHVLLVDRGLAKMARVSGRDVDFASLRHQHIGARVPVAWLESNETSCILYTSGTTGKPKGVQRDVGGYAVALATSMDTIFGGKAGGVFFCASDIGWVVGHSYIVYAPLLAGMATIVYEGLPTWPDCGVWWKIVEKYQVSRMFSAPTAIRVLKKFPTAEIRKHDLSSLEVLYLAGEPLDEPTASWVSNTLDVPVIDNYWQTESGWPIMAIARGLDDRPTRLGSPGVPMYGYNVQLLNEVTGEPCGVNEKGMLVMEGPLPPGCIQTIWGDDGRFVKTYWSLFSRPVYATFDWGIRDADGYHFILGRTDDVINVAGHRLGTREIEESISSHPGVAEVAVVGVKDALKGQVAVAFVIPKESDSLEDRDVAHSQEKAIMALVDSQIGNFGRPAHVWFVSQLPKTRSGKMLRRTIQAICEGRDPGDLTTIDDPASLDQIRQAMEE

SEQ ID NO: 2 - protein, 566 aa, Escherichia coli
MHPTGPHLGPDVLFRESNMKVTLTFNEQRRAAYRQQGLWGDASLADYWQQTARAMPDKIAVVDNHGASYTYSALDHAASCLANWMLAKGIESGDRIAFQLPGWCEFTVIYLACLKIGAVSVPLLPSWREAELVWVLNKCQAKMFFAPTLFKQTRPVDLILPLQNQLPQLQQIVGVDKLAPATSSLSLSQIIADNTSLTTAITTHGDELAAVLFTSGTEGLPKGVMLTHNNILASERAYCARLNLTWQDVFMMPAPLGHATGFLHGVTAPFLIGARSVLLDIFTPDACLALLEQQRCTCMLGATPFVYDLLNVLEKQPADLSALRFFLCGGTTIPKKVARECQQRGIKLLSVYGSTESSPHAVVNLDDPLSRFMHTDGYAAAGVEIKVVDDARKTLPPGCEGEEASRGPNVFMGYFDEPELTARALDEEGWYYSGDLCRMDEAGYIKITGRKKDIIVRGGENISSREVEDILLQHPKIHDACVVAMSDERLGERSCAYVVLKAPHHSLSLEEVVAFFSRKRVAKYKYPEHIVVIEKLPRTTSGKIQKFLLRKDIMRRLTQDVCEEIE

SEQ ID NO: 3 - protein, 399 aa, Clostridium difficile
MLLEGVKVVELSSFIAAPCCAKMLGDWGAEVIKIEPIEGDGIRVMGGTFKSPASDDENPMFELENGNKKGVSINVKSKEGVEILHKLLSEADIFVTNVRVQALEKMGIAYDQIKDKYPGLIFSQILGYGEKGPLKDKPGFDYTAYFARGGVSQSVMEKGTSPANTAAGFGDHYAGLALAAGSLAALHKKAQTGKGERVTVSLFHTAIYGMGTMITTAQYGNEMPLSRENPNSPLMTTYKCKDGRWIQLALIQYNKWLGKFCKVINREYILEDDRYNNIDSMVNHVEDLVKIVGEAMLEKTLDEWSALLEEADLPFEKIQSCEDLLDDEQAWANDFLFKKTYDSGNTGVLVNTPVMFRNEGIKEYTPAPKVGQHTVEVLKSLGYDEEKINNFKDSKVVRY

SEQ ID NO: 4 - protein, 488 aa, Aquincola tertiaricarbonus
  Aquincola tertiaricarbonus L108
MEEWNFPVEYDENYLPPADSRYWFPRRETMPAAERDKAILGRLQQVCQYAWEHAPFYRRKWEEAGFQPSQLKSLEDFEARVPVVKKTDLRESQAAHPPFGDYVCVPNSEIFHVHGTSGTTGRPTAFGIGRADWRAIANAHARIMWGMGIRPGDLVCVAAVFSLYMGSWGALAGAERLRAKAFPFGAGAPGMSARLVQWLDTMKPAAFYGTPSYAIHLAEVAREEKLNPRNFGLKCLFFSGEPGASVPGVKDRIEEAYGAKVYDCGSMAEMSPFMNVAGTEQSNDGMLCWQDIIYTEVCDPANMRRVPYGQRGTPVYTHLERTSQPMIRLLSGDLTLWTNDENPCGRTYPRLPQGIFGRIDDMFTIRGENIYPSEIDAALNQMSGYGGEHRIVITRESAMDELLLRVEPSESVHAAGAAALETFRTEASHRVQTVLGVRAKVELVAPNSIARTDFKARRVIDDREVFRALNQQLQSSAGSAWSHPQFEK

SEQ ID NO: 5 - protein, 546 aa, Pseudomonas oleovorans
MLGQMMRNQLVIGSLVEHAARYHGAREVVSVETSGEVTRSCWKEVELRARKLASALGKMGLTPSDRCATIAWNNIRHLEVYYAVSGAGMVCHTINPRLFIEQITYVINHAEDKVVLLDDTFLPIIAEIHGSLPKVKAFVLMAHNNSNASAQMPGLIAYEDLIGQGDDNYIWPDVDENEASSLCYTSGTTGNPKGVLYSHRSTVLHSMTTAMPDTLNLSARDTILPVVPMFHVNAWGTPYSAAMVGAKLVLPGPALDGASLSKLIASEGVSIALGVPVVWQGLLAAQAGNGSKSQSLTRVVVGGSACPASMIREFNDIYGVEVIHAWGMTELSPFGTANTPLAHHVDLSPDEKLSLRKSQGRPPYGVELKIVNDEGIRLPEDGRSKGNLMARGHWVIKDYFHSDPGSTLSDGWFSTGDVATIDSDGFMTICDRAKDIIKSGGEWISTVELESIAIAHPHIVDAAVIAARHEKWDERPLLIAVKSPNSELTSGEVCNYFADKVARWQIPDAAIFVEELPRNGTGKILKNRLREKYGDILLRSSSSVCE

SEQ ID NO: 6 - protein, 429 aa, Clostridium kluyveri
MEWEEIYKEKLVTAEKAVSKIENHSRVVFAHAVGEPVDLVNALVKNKDNYIGLEIVHMVAMGKGEYTKEGMQRHFRHNALFVGGCTRDAVNSGRADYTPCFFYEVPSLFKEKRLPVDVALIQVSEPDKYGYCSFGVSNDYTKPAAESAKLVIAEVNKNMPRTLGDSFIHVSDIDYIVEASHPLLELQPPKLGDVEKAIGENCASLIEDGATLQLGIGAIPDAVLLFLKNKKNLGIHSEMISDGVMELVKAGVINNKKKTLHPGKIVVTFLMGTKKLYDFVNNNPMVETYSVDYVNNPLVIMKNDNMVSINSCVQVDLMGQVCSESIGLKQISGVGGQVDFIRGANLSKGGKAIIAIPSTAGKGKVSRITPLLDTGAAVTTSRNEVDYVVTEYGVAHLKGKTLRNRARALINIAHPKFRESLMNEFKKRF

SEQ ID NO: 7 - protein, 504 aa, Rhizobium trifolii
MSNHLFDAMRAAAPGNAPFIRIDNTRTWTYDDAFALSGRIASAMDALGIRPGDRVAVQVEKSAEALILYLACLRSGAVYLPLNTAYTLAELDYFIGDAEPRLVVVASSARAGVETIAKPRGAIVETLDAAGSGSLLDLARDEPADFVDASRSADDLAAILYTSGTTGRSKGAMLTHGNLLSNALTLRDFWRVTAGDRLIHALPIFHSHGLFVATNVTLLAGASMFLLSKFDPEEILSLMPQATMLMGVPTFYVRLLQSPRLDKQAVANIRLFISGSAPLLAETHTEFQARTGHAILERYGMTETNMNTSNPYEGKRIAGTVGFPLPDVTVRVTDPATGLALPPEQTGMIEIKGPNVFKGYWRMPEKTAAEFTADGFFISGDLGKIDRDGYVHIVGRGKDLVISGGYNIYPKEVEGEIDQIEGVVESAVIGVPHPDFGEGVTAVVVRKPGAALDEKAIVSALQDRLARYKQPKRIIFAEDLPRNTMGKVQKNILRQQYADLYTRT

SEQ ID NO: 8 - protein, 660 aa, Arabidopsis thaliana
MASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWIGGHSYVTYGPMLNGATVVVFEGAPNYPDPGRCWDIVDKYKVSIFYTAPTLVRSLMRDDDKFVTRHSRKSLRVLGSVGEPINPSAWRWFFNVVGDSRCPISDTWWQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADV

SEQ ID NO: 9 - protein, 660 aa, Arabidopsis thaliana
MASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWITGHSYVTYGPMLNGATVVVFEGAPNYPDPGRCWDIVDKYKVSIFYTAPTLVRSLMRDDDKFVTRHSRKSLRVLGSVGEPINPSAWRWFFNVVGDSRCPISDTWSQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADV

SEQ ID NO: 10 - protein, 545 aa, Pseudomonas chlororaphis
MRDYEHVVESFDYLQSATQDLHGELTALNACVECCDRHAHGEAVALYCEAQDGHAERYRFRDLQRQAARFGNFLREQGVKPGDRVAGLMPRTVELLIAILGTWRIGAVYQPLFTAFGPKAIEQRLNCSNARWIVTDPHNRPKLDDVTDCPSIVVTGGAPQNPADHHFWSALNRQADDCAPVLLDASAPFLLMCTSGTTGPAKPLEVPLSAILAFKGYMRDAIDLRADDRFWNLADPGWAYGLYYAVTGPLACGYATLFYDGPFTVESTRHIIAKYAINNLAGSPTAYRFLIAAGAEFADAVRGRLRAVSSAGEPLNPQVVRWFAEQLGVVIHDHYGQTEIGMVLCNHHGLRHPVREGSAGYAVPGYRIVVLDKAHRELPAGQPGVLAVDRERSPLCWFDGYLGMPTQAFAGRYYLSGDIVELNDDGSISFVGRNDDLITTSGYRVGPFDVESALIEHPAVVEAAVIGKPDPQRTELIKAFVVLNTPYLPSPELAEELRLHVRQRLAAHAYPREMEFVDHLPKTPSGKLQRFILRNQEIAKQQALG

SEQ ID NO: 11 - protein, 485 aa, Streptomyces coelicolor
MSSLFPALSPAPTGAPADRPALRFGERSLTYAELAAAAGATAGRIGGAGRVAVWATPAMETGVAVVAALLAGVAAVPLNPKSGDKELAHILSDSAPSLVLAPPDAELPPALGALERVDVDVRARGAVPEDGADDGDPALVVYTSGTTGPPKGAVIPRRALATTLDALADAWQWTGEDVLVQGLPLFHVHGLVLGILGPLRRGGSVRHLGRFSTEGAARELNDGATMLFGVPTMYHRIAETLPADPELAKALAGARLLVSGSAALPVHDHERIAAATGRRVIERYGMTETLMNTSVRADGEPRAGTVGVPLPGVELRLVEEDGTPIAALDGESVGEIQVRGPNLFTEYLNRPDATAAAFTEDGFFRTGDMAVRDPDGYVRIVGRKATDLIKSGGYKIGAGEIENALLEHPEVREAAVTGEPDPDLGERIVAWIVPADPAAPPALGTLADHVAARLAPHKRPRVVRYLDAVPRNDMGKIMKRALNRD

SEQ ID NO: 12 - protein, 661 aa, Metallosphaera sedula
MFMRYIMVEEQTLKTGSQELEEKADYNMRYYAHLMKLSKEKPAEFWGSLAQDLLDWYEPWKETMRQEDPMTRWFIGGKINASYNAVDRHLNGPRKFKAAVIWESELGERKIVTYQDMFYEVNRWANALRSLGVGKGDRVTIYMPLTPEGIAAMLASARIGAIHSVIFAGFGSQAIADRVEDAKAKVVITADAYPRRGKVVELKKTVDEALNSLGERSPVQHVLVYRRMKTDVNMKEGRDVFFDEVGKYRYVEPERMDSNDPLFILYTSGTTGKPKGIMHSTGGYLTGTAVMLLWSYGLSQENDVLFNTSDIGWIVGHSYITYSPLIMGRTVVIYESAPDYPYPDKWAEIIERYRATTFGTSATALRYFMKYGDEYVKNHDLSSIRIIVTNGEVLNYSPWKWGLEVLGGGKVFMSHQWWQTETGAPNLGYLPGIIYMPMKSGPASGFPLPGNFVEVLDENGNPSAPRVRGYLVMRPPFPPNMMMGMWNDNGERLKKTYFSKFGSLYYPGDFAMVDEDGYIWVLGRADETLKIAAHRIGAGEVESAITSHPSVAEAAVIGVPDSVKGEEVHAFVVLKQGYAPSSELAKDIQSHVRKVMGPIVSPQIHFVDKLPKTRSGKVMRRVIKAVMMGSSAGDLTTIEDEASMDEIKKAVEELKKELKTS

SEQ ID NO: 13 - protein, 659 aa, Sulfolobus tokodaii
MTEKLSEQLQQLGEQNLEEKADYNMRYYKYLYKKSIEEPDKFWGELAEELITWYEPWKQAFVQEEGLLTKWFVGGKLNASYNAVDRHLNSHRKYKAAIFWESEKGEKKVVTYQDLFYEVNKWANALRELGVKKGDRVTIYMPLTPEGVIAKLAVARLGAIHSVVFAGFGAQALADRIADAGAKVVITADAYYRRGKLVELKKTVDEALNILGDKSPVQKVLVYKRTGTEIPFKEGRDVYFDEVGKYKYIEPVPVEATEPLFILYTSGTTGKPKGIVHSTGGYLVGTAVMLLWSYGLSQENDVLFNTSDIGWIVGHSYITYSPLVMGRSIVIYESAPDYPYPDKWAEMIEKYRATTFGTSATAIRTLMKYGEDYVKQHDLSSLRIIVTNGEPLNYAPWKWGLEVVGGGKVFMSHQWWQTETGGPNIGYIPGVVYLPMKSGPAVGFALPGNKVTVVNEEGKETKPRERGYLVMLPPFPPMMMIGMWNDPDNERLKKTYFSKFPGIYYPGDYAMIDEDGYIWVMGRADETIKVAAHRIGAGEVESIVTSHPAVAEAAAVGIPDPVKGEAVHLFVVLKVGYKPSPQLAREIQEHVRKYMGAIVTPEVHFVDKLPKTRSGKIMRRVIKAVMMGQSAGDITTLEDEASMDEIKKAVEEFKKSLSQ

SEQ ID NO: 14 - protein, 652 aa, Unknown
  Description of Unknown: S. solfataricus sequence
MVQEITENIKEIEEKVDYNTRIYREIYRESIENPGKFWGKLGEDLIDWFEPWKEIYKQETLTKWFLGGKLNASYNAIDRHLNSSRKFKAAIIWESEKGERKILTYQDLFYEVNRWANALKQLGVKKGDRVTIYMPLTPEGVTAMLACARIGAIHSVVFAGFGSQALADRIADAQSKIVITADGYYRKGRLIELKKTVDDALSKLQDNSVKNIIIFRRIGIEIPFKEGRDVFFDEIGKYKYIEPEPVEATHPLFILYTSGTTGKPKGIVHSTGGYLVGTATMLLWSYGLSQENDVLFNTSDIGWIVGHSYITYSPLVMGRSVVIYESAPDYPTPDKWAELIEKYKATTFGTSATFLRYLMKYGEDYIKAHDLSSLRIIVTNGEPLNYAPWKYGLEIIGKGRVFMSHQWWQTETGAPNLGYMPGYPIFLTMKSGPASGFPLPGNKIKVVDENGNPTRPRERGYLIIEPPFPPMMMIGMWNDDGNERVIKTYFSKFPNLYYTGDFAMIDEDGYVWVSGRADETLKIAGHRIGAGEVESAITSHPAVAEAAVIGIPDPVKGEIAHAFVVLKQGYHQNNELSKEIQEHVRKIMGPIVLLEVHFVNALPKTRSGKVMRRVIKAVMTGSNIGDISTLEDEASMEEIKKAIEVLRRQLNP

SEQ ID NO: 15 - protein, 705 aa, Unknown
  Description of Unknown: Nitrosopumilus maritimus sequence
MAAVKKIFDEIIETDHKVITEESSKSILKNYGVKVPPYALVTSAEEAAKEAKKIGFPLVMKVVSPQILHKTDVGGVKVGLDNVADVKKTFTDMYGRLSKKKGVNVKGILLEKMVPKGVELIVGIQNDSQFGPIIMVGMGGIMTEVMKDVAFRMLPITTSDAKSMLNELKGSKLLKGFRGSEPIDTNLVAKMLVNIGKLGVENADYINSIDFNPVIVYPKSHYVVDAKIILNKEKKKNSISKAKPSITDMETFFTPKSVALVGASASPGKIGNSILDSLVNYDFKGKVYPINPKADKIFGQKCYPSVADIPGKVDLVVVSVDLSMTPPVLEDCAKKGVHSVVIVSGGGKELGGERAAYEAEVARLSKKHKIRIIGPNCIGMFNAANRLDCAFQGQERMVRSKLGPVAFFSQSGTMGISMLESADTFGLSKMISFGNRSDVDEADMIWYAANDPQTKVIGLYVEGFGDGRKFINVAKRVMKEKKKPIVIWKSGRTAAGAKQAASHTGSLGGSNAIIMGAFKQAGIISVDSYQELAGVLKALAWQPAAKGNKVAMTSNGAGPMIGGIDQLEKFGLAIGKLSPKLLKKMKSRFPPAVPIHNGNPADVGGGATADDYQFVIQQFMDEKNIDIAMPWFVFQDDPLEETIVDHLAGFQKKAKKPLLCGGNGGPYTEKMIKLIEKHNVPVYQDLRTWVAAASALHQWGKISKK

SEQ ID NO: 16-18 - skipped (3 entries)

SEQ ID NO: 19 - protein, 589 aa, Cupriavidus necator
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 20 - protein, 567 aa, Chromobacterium sp.
  Chromobacterium USM2
MQQFVNSLSLGQDQSDAPHPLTGAWSQLMSQTNQLLQLQSSLYQQQLGLWTQFLGQTAGNDASAPSAKPSDRRFASPEWDEHPFYSFLKQSYLQTSKWMMELVDKTQIDESAKDKLSFATRQYLDAMAPSNFMLTNPDVVKRAIETQGESLVEGMKNMMEDIQKGHISMSDESKFQIGKNLVVTPGEVVFRNELIELIQYTPTTEKVHEKPLLFVPPCINKYYLMDLQPDNSMVRHFVGQGYRVFLVSWRSAVPEMKNFTWETYIEKGVFAAAEAVQKITKQPTMNALGFCVGGVILTTALCVAQAKGLKYFDSATFMTSLIDHAEPGEISFFIDEALVASREAKMAAGGIISGKEIGRTFASLRANDLVWNYVVNNYLLGKTPAPFDLLYWNNDAVDLPLPMHTFMLRQFYINNALITPGAITLCGVPIDISKIDIPVYMFAAREDHIVLWSSAYSGLKYLSGTPSRRFVLGASGHIAGSINPVTKDKRNYWTNEQLPVNPEEWLEGAQSHPGSWWKDWDAWLAPQSGKQVPAPKMLGSKEFPPLQPAPGSYVLAKAMPPVAAALN

SEQ ID NO: 21 - protein, 559 aa, Pseudomonas sp.
  Pseudomonas sp. MBEL 6-19
MSNKSNDELKYQASENTLGLNPVVGLRGKDLLASARMVLRQAIKQPVHSVKHVAHFGLELKNVLLGKSGLQPTSDDRRFADPAWSQNPLYKRYLQTYLAWRKELHDWIDESNLAPKDVARGHFVINLMTDAMAPTNTAANPAAVKRFFETGGKSLLDGLSHLAKDLVHNGGMPSQVNMGAFEVGKSLGVTEGAVVFRNDVLELIQYKPTTEQVYERPLLVVPPQINKFYVFDLSPDKSLARFCLRNNVQTFIVSWRNPTKEQREWGLSTYIEALKEAVDVVTAITGSKDVNMLGACSGGITCTALLGHYAAIGENKVNALTLLVTVLDTTLDSDVALFVNEQTLEAAKRHSYQAGVLEGRDMAKVFAWMRPNDLIWNYWVNNYLLGNEPPVFDILFWNNDTTRLPAAFHGDLVELFKNNPLIRPNALEVCGTPIDLKQVTADIFSLAGTNDHITPWKSCYKSAQLFGGNVEFVLSSGGHIKSILNPPGNPKSRYMTSTEVAENADEWQANATKHTDSWWLHWQAWQAQRSGELKKSPTKLGSKAYPAGEAAPGTYVHER

SEQ ID NO: 22 - protein, 355 aa, Allochromatium vinosum
MFPIDIRPDKLTQEMLDYSRKLGQGMENLLNAEAIDTGVSPKQAVYSEDKLVLYRYDRPEGAPEAQPVPLLIVYALVNRPYMTDIQEDRSTIKGLLATGQDVYLIDWGYPDQADRALTLDDYINGYIDRCVDYLREAHGVDKVNLLGICQGGAFSLMYSALHPDKVRNLVTMVTPVDFKTPDNLLSAWVQNVDIDLAVDTMGNIPGELLNWTFLSLKPFSLTGQKYVNMVDLLDDPDKVKNFLRMEKWIFDSPDQAGETFRQFIKDFYQNNGFLNGGVVLGGQEVDLKDITCPVLNIFALQDHLVPPDASRALKGLTSSPDYTELAFPGGHIGIYVSGKAQKEVTPAIGKWLNER

SEQ ID NO: 23 - protein, 357 aa, Allochromatium vinosum
MSNTNFFNDDWLELQRKYWDNWTDMSRKAMGLDSASSSATTPWEAAIDQWWKAMAPAAPDLSRSFMEKMMEQGKNFFRLADTFAKRADEGNAGNGLELWTKTLEDMQKRFSGSLDDGGNTMQRLMSFWELPLDNWQRMMSSMSPMPGDMLRNMPHEQFKDSLDRALSAPGLGYTREEQSQYQELMRSAMEYQAALQEYTNVYTKLGMKSVEHMGSYIQGVIDSGKTIDSARALYDNWVACCEGAYADEVATPEYARIHGRLVNAQMALKKRMSILVDENLGALNMPTRSELRTLQDRLQETRRENKALRHSLHSLERRVAALAGEEPATKPATALRSPAPAAKAPARRRTTKTNPAD

SEQ ID NO: 24 - protein, 357 aa, Thiococcus pfennigii
MSPFPIDIRPDKLTEEMLEYSRKLGEGMQNLLKADQIDTGVTPKDVVHREDKLVLYRYRRPAQVATQTIPLLIVYALVNRPYMTDIQEDRSTIKGLLATGQDVYLIDWGYPDQADRALTLDDYINGYIDRCVDYLRETHGVDQVNLLGICQGGAFSLCYTALHSEKVKNLVTMVTPVDFQTPGNLLSAWVQNVDVDLAVDTMGNIPGELLNWTFLSLKPFSLTGQKYVNMVDLLDDEDKVKNFLRMEKWIFDSPDQAGETFRQFIKDFYQRNGFINGGVLIGDQEVDLRNIRCPVLNIYPMQDHLVPPDASKALAGLTSSEDYTELAFPGGHIGIYVSGKAQEGVTPAIGRWLNERG

SEQ ID NO: 25 - protein, 367 aa, Thiococcus pfennigii
MNDTANKTSDWLDIQRKYWETWSELGRKTLGLEKTPANPWAGALDHWWQTVSPAAPNDLVRDFMEKLAEQGKAFFGLTDYFTKGLGGSSGTQGWDTLSKTIDDMQKAFASGRIEGDETFRRLMAFWEMPLDNWQRTMSSLSPVPGDLLRNMPHDQVRDSVDRILSAPGLGYTREEQARYQDLIRRSLEYQSALNEYNGFFGQLGVKSLERMRAFLQGQAEKGVAIESARTLYDAWVGCCEEVYAEEVSSADYAHIHGRLVNAQMALKQRMSTMVDEVLGAMPLPTRSELRTLQDRLQESRGEGKRQRQEIETLKRQVAALAGGAQPAPQASAQPSTRPAPATAPAASAAPKRSTTTRRKTTKPTTGQ

SEQ ID NO: 26 - protein, 362 aa, Bacillus megaterium
MAIPYVQEWEKLIKSMPSEYKSSARRFKRAYEIMTAEAEPEVGLTPKEVIWKKNKAKLYRYTPVKDNLHKTPILLVYALINKPYILDLTPGNSLVEYLLNRGFDVYLLDWGTPGLEDSNMKLDDYIVDYIPKAAKKVLRTSKSPDLSVLGYCMGGTMTSIFAALNEDLPIKNLIFMTSPFDFSDTGLYGAFLDDRYFNLDKAVDTFGNIPPEMIDFGNKMLKPITNFYGPYVTLVDRSENQRFVESWKLMQKWVADGIPFAGEAYRQWIRDFYQQNKLINGELEVRGRKVDLKNIKANILNIAASRDHIAMPHQVAALMDAVSSEDKEYKLLQTGHVSVVFGPKAVKETYPSIGDWLEKRSK

SEQ ID NO: 27 - protein, 199 aa, Bacillus megaterium
MEQQKVFDPFQAWKDVYDKTESYWGKVIGDNMNREEFSQLMGNVLNMNLQYQQAVNEVTGRYLHQVNVPTKEDVANVASLVINVEEKVELLEEQFDDRFGELEAQQESASALKKDVTKLKSDVKSLDKKLDKVLSLLEGQQKTQDELKETIQKQIKTQGEQLQAQLLEKQEKLAEKPKAEAKSEAKPSNAQKTEQPARK

SEQ ID NO: 28 - protein, 358 aa, Unknown
  Description of Unknown: Candidatus Accumulibacter phophatis clade IIA str. UW-1 sequence
MENKNPNEQLGGVVNAWADMQKRMWGDWSSLLQNLPGGSEGPVEAAKKGVAAASKGTNEAARMLMDRMTSSQGAMNRVMDFFFKSMKIVAPNLEANKDWRPDLKGFAEQWAKESTAMLERSFGMGSHLGNLSSTLSKDLPDAMGPWLSFLMQAASSGHVGEAMLGGTSGINRLLSMEGDAALAGVGEIPLFGASREKNAKLLRLVDAVVDLRKNSLTFHTAFGDALAKAVEATVEELGKVAAKGEKITAVRQLMSLWYRTADKSLLVTFNTQEFLDKQNAFTAAQQQFKLAQRAVVEDIFRGLDMPTRSELDETYQVIHELKKEVRALKKALLPAAPAAVAKSSPAPRKAAAARAKSE

SEQ ID NO: 29 - protein, 368 aa, Unknown
  Description of Unknown: Candidatus Accumulibacter phophatis clade IIA str. UW-1 sequence
MFSFPIQILPADVAAETAALNEKLAKGISNLTNLTDDDIDIGSTPKDVVFEQDGIKVYHYHALAEPSQIMKTPLLIVPPLINGYEVADLQPDRSLVRNLLNQGIDVYLNDWGYPRQVDKYRTLDDYINGYFDDTVDFIRKHHGVDKIALFGICQGGAMSTTYSTLNPDKISHLVLTVSPIDFDAYKANHKPHEGLMFTMGADADVEKMVAVHGNVPATVLNESFMMASPFILNYGKYADVIDILDDRLALQNFLRMEKWLFGGPDAGGQMFKEFIRDFLKGNKLVKGTLEIGGRKVDLKELTIPILNIFAEKDHIVPPPCTVALGKHVGSKDYTEFAIATGHIGIYTGGLSQKVLAPTVGKWMRERGA

SEQ ID NO: 30 - protein, 594 aa, Aeromonas caviae
MSQPSYGPLFEALAHYNDKLLAMAKAQTERTAQALLQTNLDDLGQVLEQGSQQPWQLIQAQMNWWQDQLKLMQHTLLKSAGQPSEPVITPERSDRRFKAEAWSEQPIYDYLKQSYLLTARHLLASVDALEGVPQKSRERLRFFTRQYVSAMAPSNFLATNPELLKLTLESGGQNLVRGLALLAEDLERSADQLNIRLTDESAFELGRDLALTPGRVVQRTELYELIQYSPTTETVGKTPVLIVPPFINKYYIMDMRPQNSLVAWLVAQGQTVFMISWRNPGVAQAQIDLDDYVVDGVIAALDGVEAATGEREVHGIGYCIGGTALSLAMGWLAARRQKQRVRTATLFTTLLDFSQPGELGIFIHEPIIAALEAQNEAKGIMDGRQLAVSFSLLRENSLYWNYYIDSYLKGQSPVAFDLLHWNSDSTNVAGKTHNSLLRRLYLENQLVKGELKIRNTRIDLGKVKTPVLLVSAVDDHIALWQGTWQGMKLFGGEQRFLLAESGHIAGIINPPAANKYGFWHNGAEAESPESWLAGATHQGGSWWPEMMGFIQNRDEGSEPVPARVPEEGLAPAPGHYVKVRLNPVFACPTEEDAA

SEQ ID NO: 31 - protein, 609 aa, Unknown
  Description of Unknown: Casimicrobium sp. Sequence
MSTKKPGEAGNNPFAAWMENNPFAAAAANNPFAAGSADNPFAGGGKAGANPFAAGLDAMQTMMSGNLGQQPWAASAMPSFAGAPAAPEAGWWWLPAVKPEAFQAAAMKWFSPQTDVAALAAADRRFRGEGWQSQPFFQAVRDQYLRTAAFWRDVVEHADLDDRERHRAKFFLEQFLDASAPSNFFLTNPEAIQRAVETKGESVKHGMENLAHDIEAGHIAMTDETAFKVGENLAVTPGQVVFRNELIEIIHYTPTQKTVYQRPLLIVPPCINKFYILDLKPENSFVAHAVAQGFNVYLVSWRNVPEELKTLTWEDYLEEGALTAIDEVRSHAGIEKINVLGFCVGGTILASALGVLAARGELDDFIESATYLTTLLDFSEPGDIKAYLGESTYQMRAQQFGPDGTGGMMKGSELAQSFASLRANDLIWNYVVNNYLKGQDPPAFDLLFWNGDSTNLPGPMYLYYIRNFYLDNKLTEHGGINMLGEDVDLALVDVPTFVYCSREDHIVPWKSAFASAELWGGDVEFVMGASGHIAGVINPPGPKKRSYWTGNFPAPTPEAWDSKAKEHPGSWWPHWYTWLAPHSGKRVAAPKQPKSKLGAAPGTYVLAKA

SEQ ID NO: 32 - protein, 622 aa, Unknown
  Description of Unknown: Burkholderiales sequence
MSSKKSSTKGDAAGAQRAENPFAAWASGNPFLSGVANPFANAAQDATAKFAEMMNAAPGANPFAEGIQAMQTLMSSNIGQQPSIVSTVPAFAQPGPMPMPEAGWWWLPSVQPTVWQQALTTWFSPQTDVAALAAADRRFRSKSWESQPFFQLARDHYLRNCAFWREVVSKADLDDRERHRARFFVEQVLDATAPTNFFLTNPEAIERAIETKGESVKHGIENLSHDIEAGHIAMTDEKAFKVGENLAVTPGQVVFRNELIELIHYTPTEKTVYQRPLLIVPPCINKFYILDLKPENSFVAHAVAQGFDVYLVSWRNVGDDLKALTWEDYLEEGVLTAIDETRDHSGAATINTLGFCVGGTILSCALAVLASRGELDDFVESATYLTTLLDFTEPGDIKAYLGESTYQMRVQQFGPDGAGGMMKGSELAQSFASLRANDLIWNYVVNNYLKGQDPPAFDLLYWNGDSTNLPGPMYLYYLRNFYLDNKLTKPGTLDMIGEPVDLSNVDIPTYVYCSREDHIVPWKSAFASAELWGGDVEFVVGASGHIAGVINPPGPKKRSYWTGRWPADTPEAWDAKASEHAGSWWPHWYAWLAPHSGKRVPAKKQGKSPLGAAPGQFVLEKA

SEQ ID NO: 33 - protein, 573 aa, Rhizobacter sp.
MSTESQIPPSLQSLMSSAWGAAAAPSFPGMPTMPGMPTLPIGPGMPTMAAPEAGWWWLPAVNPGAMQAAATRWFSPETDVSALAASDRRFRAPSWQAQPYFQATRDQYLRTAAYWRELVAHADLEEKERHRAKFFLEQMLDAVAPSNFFLTNPEAIERAIETKGESLRHGIENLKHDIETGHIAMTDETVFQVGGNLALTPGQVVFRNDLIELLHYTPTQKTLHSRPLLIVPPCINKFYILDLKPENSFVGHAVAQGFDVYLVSWRNIPEALRALTWEDYLEQGVLTAIDEVREHAGVDTINTLGFCVGGTILSCALAVLAARGELDDFVESATYLTTLLDFSEPGDIKAYLGESTFKLREQQLNKDGDTGLMKGSELAQSFASLRANDLIWNYVVNNYLKGQDPPAFDLLYWNGDSTNLPGPMYLYYMRNFYLDNKLMEPDALTMLDEPIDLSSVNIATYVYCSREDHIVPWKSAYASAELWGGEVEFVLGASGHIAGVVNPPGPKKRSYWTGRWPAASTDEWDAKAKETPGSWWPHWYAWLAERSGKKVVKKSPSKSKLGAAPGSYVLEKM

SEQ ID NO: 34 - protein, 577 aa, Unknown
  Description of Unknown: Betaproteobacteria sequence
MDFSDYTKAHQAWQEFLSKNAGAMGGMGAGAAGMPSFAFPNAQFAFPSGAGSMPQLEAGWWWLPSVKPEAWQAAAKWYALDTKLDDLIAKDRRFRSEAWTKQPFFQGIRDQYVRTAAFWRDLLQGAALDDKEKQRARFFLEQWLDAIAPTNFFATNPEAIEKAIETKGESLKHGIENLVHDIERGHIAMTDESAFAVGKNVALSKGEVIFRNDLIELIHYAPTQKTVHERPLLIVPPCINKFYILDLKPENSFVGHAVAAGFDVYLVSWRNIPEALKTLVWEDYLESGVLTAIDEVREHASVETINTLGFCVGGTILACSLAVLAARGELEEFVESATYLTTLLDFSEPGDIQAYLGESTYAMRVKQLGEDGTNGLMSGAELAQAFASLRANDLIWSFVVKNYLKGEDPPAFDLLYWNSDSTNLPGPMYLYYLRNFYLDNKLTQPGALTMLEEPIDLSLVTIDSYVYSSREDHIVPWKSAFASAELWGGDVEFVLGASGHIAGVVNPPIPVKRSYWTSAWPAENPEAWDAKSKEHPGSWWPHWYTWLAKRSGKKSAPRKHKVSKLGAAPGQYVLEKI

SEQ ID NO: 35 - protein, 579 aa, Blastococcus sp.
MSSPSPDPAKWLRDLMQTEPAALWPAVNIADTGKQVAAVAAPWTKAVADFTAMQLTAVQQMTAPWTAALPGLGAAAEPVKDKRFAGDEWTKDPRYEAVVRTYLTQSDLLHKALDAAPLDEHSKAQWGFALRQVIDALSPANTLATNPEAMQLAMETGGASLVDGLQLFTEDLAKGRVSMTDEMAFEVGRDVGTTPGGVVYQNDLMQLIQYTPTTAKVHKRPLVIVPPCINKFYILDLRPTNSFVAHAVAQGHTVLLVSWRNAGPAQDRLTWDDYLEQGVLKAIDVARSITKADKVNTLGFCVGGTLLASALAVQAARGEQPAASMTLLTTMLDFTDTGEIGVLVTEPAVVAREAAIGRGGLLKGSELGQVFASLRANDLIWPYVVKGYLQGQAPPAFDMLFWNGDETNLPGPMFCWYVRNAYLENKLREPGGTVQLGQPVDLAAVDVPAFVYASKEDHIVPWQTAYASTQILSGDTTFVLGASGHIAGVINPPAANKRNYWTRPDPEGTDGAEGPVPLDADPDRWFEAAERVPGSWWPAWAAWLTPHAGPQVAARKKLGNAEFAVLEEAPGSYVREPAS

SEQ ID NO: 36 - protein, 597 aa, Burkholderia cepacia
MFESWLNAWRGFADPARAATASAAVNPFATFQFPASFPFQMPSMPDLGGLAGLGGMASPFAGLKLPVAAIPPERLQALQADYARDCMTLMQQAAAAKLESPELKDRRFSGDAWKSSPAHAFAAAWYLLNARYLQELADSLQTDPKTRERIRFTVQQWTAAAAPSNFLALNPDAQKSILDTQGESLRQGMMNLLGDLQRGKISQTDESQFVVGKNLGCTEGAVVYENDLIPADPSTRRRRTRCFERPLLIVSAVHQQVLHPRPAAREFASSRTRCRTVIRCFLVSWRNADASVAHKTWDDYMNEGLLAAIDAVQQVSGREQINTLGFCVGGTMLATALAVLAARGEHPAASMTLLTAMLDFSDTGILDVFVDEAHVQMREQTIGGKNGTQPGLMRGVEFANTFSFLRPNDLVWNYVVDNYLKGRTPAPFDLLYWNSDSTSLPGPMYAWYLRNTYLENKLREPGALTVCGESVDLSLIDVPTFIYGSREDHIVPWQTAYASTSILSGPLKFVLGASGHIAGVINPPAKKKRSYWVNDDLPESADDWFAGATEQPGSWWPTWVEWLDAYGGRKVAPPAEAGSAQFPVIEPAPGRYVLQRD

SEQ ID NO: 37 - protein, 562 aa, Rhodococcus ruber
MLDHVHKKLKSTLDPIGWGPAVTSVAGRAVRNPQAVTAATAEYTGRLAKIPAAATRVFNANDPDAPMPVDPRDRRFSDTAWQENPAYFSLLQSYLATRAYVEELTEAGSGDPLQDGKARQFANLMFDALAPSNFLWNPGVLTRAFETGGASLLRGARYAAHDILNRGGLPLKVDSDAFTVGENLAATPGKVVFRNDLIELIQYAPQTEQVHAVPILAAPPWINKYYILDLAPGRSLAEWAVQHGRTVFMISYRNPDESMRHITMDDYYVDGIATALDVVEEITGSPKIEVLSICLGGAMAAMAAARAFAVGDKRVSAFTMLNTLLDYSQVGELGLLTDPATLDLVEFRMRQQGFLSGKEMAGSFDMIRAKDLVFNYWVSRWMKGEKPAAFDILAWNEDSTSMPAEMHSHYLRSLYGRNELAEGLYVLDGQPLNLHDIACDTYVVGAINDHIVPWTSSYQAVNLLGGDVRYVLTNGGHVAGAVNPPGKRVWFKAVGAPDAESGTPLPADPQVWDEAATRYEHSWWEDWTAWSNKRAGELVAPPAMGSTAHPPLEDAPGTYVFS

SEQ ID NO: 38 - protein, 565 aa, Rhodococcus opacus
  Rhodococcus opacus 1
MSDTPLPTIPDELTAPLDLLLTSGSRSVAARMLPDSSWTRLGVGLAGRPGTVARRGGALVRELGAIAAGTSDRTPAKSDKRFGDAAWQQNPALRRAMQAYLATSHTAAALLDDAELDWRDHERMRFVLDNLVEGLSPTNNPLLSPLGWKAMVDTGGLSAARGVRALVRDMLSKPRVPAMVEPDAFAVGEDVAATKGAVVLQTRTFELIHYTPQTEKVHATPLLIVPPVINKYYILDIAPGRSLIEYLLQQGQQVFAISWRNPHARHRDWDADTYGSAIVEALDTVQCVAGTDSAHVLGTCSGGILAAMVAAHLTEIGEGDRIAGLTLAVTVLDQTQAGTAAAVMSERAAAAAIRDSAARGYLDGRTLAEMFAWLRPSDLVWRYWVNNYVQGRAPAAFDVLFWNSDTTRMTAALHRDLVLLGLRNALTAPGAATMLGTPVDLSTVAADAYVVGGSADHLCPWQSTYRSARLLGSKDSRFVLSSNGHIASLVNPPGNPRASFRFGQPVPETPDEWLAAAETASDSWWPDYARWLAERSGPDVDAPHGLGARQFPPLAPAPGTYVHHS

SEQ ID NO: 39 - protein, 355 aa, Rhodococcus opacus
  Rhodococcus opacus 2
MLDIAWNPTLNTLTRNAWRLSFGGGIAPVQRTPSTRIHEAPHQDLYRFDSTETDGGRPVLLVPPLAAPAHCFDLRPGQSLAAHLVGSGKAAYLVDYGTMGYSDRGLGFEDWIDDFIPTAVDRVSRLHDGAPVDLIGWSLGGTMSLLTAAGRNYLPIGSVTAIGTPIDYESVTMIAPLRMVGRFTGDRPLTTATRAMGGLPAPLVQASYRFTALQRELTKPWFIARNLHDTETLARMESIDRFMADMPGYPARFFRQVCTELILGNALAAGSFQTRNGAIALADLAVPVLAIGGTEDVIAPIPSVRAATDVLTGSPSVRFESAPGSHLGLVAGPKAKDSTWAHIDGFLDGVVAQTA

SEQ ID NO: 40 - protein, 363 aa, Rhodococcus opacus
  Rhodococcus opacus 3
MSPIGPVVDIARGLVREVERTQLRARRGIELIARRPPPQVSNTPKDEVWSFGKAKLWRYRNDDVRHGPPVLMFLGLVGDSAIFDLFPGNSWAEKLVAEGFDVFLFDWGRPEAAEGEHDLGTYMDGYFVPAVDAVRRIAGADEVSVGAYCMGSLMLTLLLGSRSNVPVRNAVLFAPPCDYDHAPGFLTGFKDGRLETRHVVDEMTGLVPEDAVRGMFRLLQPTSDIVQYVTLWENLWRDGYADAHRAINHWAWDHRSMAAPSFVEMVEDYVRENRLVNGGAELAGRPVDLHSITIPLLMIIAEKDEFVPPANSEPLAELVGSDDVEILRIPGGHAGALMGSAARKKTMPGVVDWLRRHSDTPPR

SEQ ID NO: 41-43 - skipped (3 entries)

SEQ ID NO: 44 - protein, 140 aa, Unknown
  Description of Unknown: uncultured bacterium sequence
DEVRHISNGYATLLMALADEGNHQLLARDLRYAWWNNHRVVDAAIGTFIEYGTRDRRKDRESYAEMWRRWIYDDYYRSYLVPLEKYGLEIPHDLIEEAWNQIWNKGYVHEVAQFFATGWLANYWRIDPMTDKDFEWFENK

SEQ ID NO: 45 - protein, 141 aa, Methylococcus capsulatus
  Methylococcus capsulatus (Bath)
MSVNSNAYDAGIMGLKGKDFADQFFADENQVVHESDTVVLVLKKSDEINTFIEEILLTDYKKNVNPTVNVEDRAGYWWIKANGKIEVDCDEISELLGRQFNVYDFLVDVSSTIGRAYTLGNKFTITSELMGLDRKLEDYHA

SEQ ID NO: 46 - protein, 348 aa, Methylococcus capsulatus
  Methylococcus capsulatus (Bath)
MQRVHTITAVTEDGESLRFECRSDEDVITAALRQNIFLMSSCREGGCATCKALCSEGDYDLKGCSVQALPPEEEEEGLVLLCRTYPKTDLEIELPYTHCRISFGEVGSFEAEVVGLNWVSSNTVQFLLQKRPDECGNRGVKFEPGQFMDLTIPGTDVSRSYSPANLPNPEGRLEFLIRVLPEGRFSDYLRNDARVGQVLSVKGPLGVFGLKERGMAPRYFVAGGTGLAPVVSMVRQMQEWTAPNETRIYFGVNTEPELFYIDELKSLERSMRNLTVKACVWHPSGDWEGEQGSPIDALREDLESSDANPDIYLCGPPGMIDAACELVRSRGIPGEQVFFEKFLPSGAA

SEQ ID NO: 47 - protein, 103 aa, Methylococcus capsulatus
  Methylococcus capsulatus (Bath)
MVESAFQPFSGDADEWFEEPRPQAGFFPSADWHLLKRDETYAAYAKDLDFMWRWVIVREERIVQEGCSISLESSIRAVTHVLNYFGMTEQRAPAEDRTGGVQH

SEQ ID NO: 48 - protein, 527 aa, Methylococcus capsulatus
  Methylococcus capsulatus (Bath)
MALSTATKAATDALAANRAPTSVNAQEVHRWLQSFNWDFKNNRTKYATKYKMANETKEQFKLIAKEYARMEAVKDERQFGSLQDALTRLNAGVRVHPKWNETMKVVSNFLEVGEYNAIAATGMLWDSAQAAEQKNGYLAQVLDEIRHTHQCAYVNYYFAKNGQDPAGHNDARRTRTIGPLWKGMKRVFSDGFISGDAVECSLNLQLVGEACFTNPLIVAVTEWAAANGDEITPTVFLSIETDELRHMANGYQTVVSIANDPASAKYLNTDLNNAFWTQQKYFTPVLGMLFEYGSKFKVEPWVKTWNRWVYEDWGGIWIGRLGKYGVESPRSLKDAKQDAYWAHHDLYLLAYALWPTGFFRLALPDQEEMEWFEANYPGWYDHYGKIYEEWRARGCEDPSSGFIPLMWFIENNHPIYIDRVSQVPFCPSLAKGASTLRVHEYNGQMHTFSDQWGERMWLAEPERYECQNIFEQYEGRELSEVIAELHGLRSDGKTLIAQPHVRGDKLWTLDDIKRLNCVFKNPVKAFN

SEQ ID NO: 49 - protein, 389 aa, Methylococcus capsulatus
  Methylococcus capsulatus (Bath)
MSMLGERRRGLTDPEMAAVILKALPEAPLDGNNKMGYFVTPRWKRLTEYEALTVYAQPNADWIAGGLDWGDWTQKFHGGRPSWGNETTELRTVDWFKHRDPLRRWHAPYVKDKAEEWRYTDRFLQGYSADGQIRAMNPTWRDEFINRYWGAFLFNEYGLFNAHSQGAREALSDVTRVSLAFWGFDKIDIAQMIQLERGFLAKIVPGFDESTAVPKAEWTNGEVYKSARLAVEGLWQEVFDWNESAFSVHAVYDALFGQFVRREFFQRLAPRFGDNLTPFFINQAQTYFQIAKQGVQDLYYNCLGDDPEFSDYNRTVMRNWTGKWLEPTIAALRDFMGLFAKLPAGTTDKEEITASLYRVVDDWIEDYASRIDFKADRDQIVKAVLAGLK

SEQ ID NO: 50 - protein, 170 aa, Methylococcus capsulatus
  Methylococcus capsulatus (Bath)
MAKLGIHSNDTRDAWVNKIAQLNTLEKAAEMLKQFRMDHTTPFRNSYELDNDYLWIEAKLEEKVAVLKARAFNEVDFRHKTAFGEDAKSVLDGTVAKMNAAKDKWEAEKIHIGFRQAYKPPIMPVNYFLDGERQLGTRLMELRNLNYYDTPLEELRKQRGVRVVHLQSPH

SEQ ID NO: 51-53 - skipped (3 entries)

SEQ ID NO: 54 - protein, 345 aa, Corynebacterium glutamicum
MTTAAPQEFTAAVVEKFGHDVTVKDIDLPKPGPHQALVKVLTSGICHTDLHALEGDWPVKPEPPFVPGHEGVGEVVELGPGEHDVKVGDIVGNAWLWSACGTCEYCITGRETQCNEAEYGGYTQNGSFGQYMLVDTRYAARIPDGVDYLEAAPILCAGVTVYKALKVSETRPGQFMVISGVGGLGHIAVQYAAAMGMRVIAVDIADDKLELARKHGAEFTVNARNEDSGEAVQKYTNGGAHGVLVTAVHEAAFGQALDMARRAGTIVFNGLPPGEFPASVFNIVFKGLTIRGSLVGTRQDLAEALDFFARGLIKPTVSECSLDEVNGVLDRMRNGKIDGRVAIRF

SEQ ID NO: 55 - protein, 506 aa, Corynebacterium glutamicum
MTVYANPGTEGSIVNYEKRYENYIGGKWVPPVEGQYLENISPVTGEVFCEVARGTAADVELALDAAHAAADAWGKTSVAERALILHRIADRMEEHLEEIAVAETWENGKAVRETLAADIPLAIDHFRYFAGAIRAQEDRSSQIDHNTVAYHFNEPIGVVGQIIPWNFPILMATWKLAPALAAGNAIVMKPAEQTPASILYLINIIGDLIPEGVLNIVNGLGGEAGAALSGSNRIGKIAFTGSTEVGKLINRAASDKIIPVTLELGGKSPSIFFSDVLSQDDAFAEKAVEGFAMFALNQGEVCTCPSRALVHESIADEFLELGVKRVQNIKLGNPLDTETMMGAQASQEQMDKISSYLKIGPEEGAQTLTGGKVNKVDGMENGYYIEPTVFRGTNDMRIFREEIFGPVLSVATFSDFDEAIRIANDTNYGLGAGVWSRDQNTIYRAGRAIQAGRVWVNQYHNYPAHSAFGGYKESGIGRENHLMMLNHYQQTKNLLVSYDPNPTGLF

SEQ ID NO: 56 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 57 - protein, 714 aa, Escherichia coli
MSNVQEWQQLANKELSRREKTVDSLVHQTAEGIAIKPLYTEADLDNLEVTGTLPGLPPYVRGPRATMYTAQPWTIRQYAGFSTAKESNAFYRRNLAAGQKGLSVAFDLATHRGYDSDNPRVAGDVGKAGVAIDTVEDMKVLFDQIPLDKMSVSMTMNGAVLPVLAFYIVAAEEQGVTPDKLTGTIQNDILKEYLCRNTYIYPPKPSMRIIADIIAWCSGNMPRFNTISISGYHMGEAGANCVQQVAFTLADGIEYIKAAISAGLKIDDFAPRLSFFFGIGMDLFMNVAMLRAARYLWSEAVSGFGAQDPKSLALRTHCQTSGWSLTEQDPYNNVIRTTIEALAATLGGTQSLHTNAFDEALGLPTDFSARIARNTQIIIQEESELCRTVDPLAGSYYIESLTDQIVKQARAIIQQIDEAGGMAKAIEAGLPKRMIEEASAREQSLIDQGKRVIVGVNKYKLDHEDETDVLEIDNVMVRNEQIASLERIRATRDDAAVTAALNALTHAAQHNENLLAAAVNAARVRATLGEISDALEVAFDRYLVPSQCVTGVIAQSYHQSEKSASEFDAIVAQTEQFLADNGRRPRILIAKMGQDGHDRGAKVIASAYSDLGFDVDLSPMFSTPEEIARLAVENDVHVVGASSLAAGHKTLIPELVEALKKWGREDICVVAGGVIPPQDYAFLQERGVAAIYGPGTPMLDSVRDVLNLISQHHD

SEQ ID NO: 58 - protein, 1220 aa, Chloroflexus aurantiacus
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 59-61 - skipped (3 entries)

SEQ ID NO: 62 - protein, 548 aa, Escherichia coli
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM

SEQ ID NO: 63 - protein, 97 aa, Escherichia coli [sequence omitted]

SEQ ID NO: 64 - protein, 559 aa, Methylococcus capsulatus
MAKEVVYRGSARQRMMQGIEILARAAIPTLGATGPSVMIQHRADGLPPISTRDGVTVANSIVLKDRVANLGARLLRDVAGTMSREAGDGTTTAIVLARHIAREMFKSLAVGADPIALKRGIDRAVARVSEDIGARAWRGDKESVILGVAAVATKGEPGVGRLLLEALDAVGVHGAVSIELGQRREDLLDVVDGYRWEKGYLSPYFVTDRARELAELEDVYLLMTDREVVDFIDLVPLLEAVTEAGGSLLIAADRVHEKALAGLLLNHVRGVFKAVAVTAPGFGDKRPNRLLDLAALTGGRAVLEAQGDRLDRVTLADLGRVRRAVVSADDTALLGIPGTEASRARLEGLRLEAEQYRALKPGQGSATGRLHELEEIEARIVGLSGKSAVYRVGGVTDVEMKERMVRIENAYRSVVSALEEGVLPGGGVGFLGSMPVLAELEARDADEARGIGIVRSALTEPLRIIGENSGLSGEAVVAKVMDHANPGWGYDQESGSFCDLHARGIWDAAKVLRLALEKAASVAGTFLTTEAVVLEIPDTDAFAGFSAEWAAATREDPRV

SEQ ID NO: 65 - protein, 96 aa, Methylococcus capsulatus
MKIRPLHDRVIIKRLEEERTSAGGIVIPDSAAEKPMRGEILAVGNGKVLDNGEVRALQVKVGDKVLFGKYAGTEVKVDGEDVVVMREDDILAVLES

SEQ ID NO: 66 - protein, 96 aa, Methylococcus capsulatus
MKIRPLHDRVVVIRREEEKTSPGGIVIPDTAKEKPIKGEIVAVGTGKVLDNGQVRPLAVKAGDTVLFGKYSGTEIKIDGTEYLMLREDDIMGVIES

(19) United States
(12) Patent Application Publication
(10) Pub. No.: US 2023/0407350 A1
(43) Pub. Date: Dec. 21, 2023

(54) MICROORGANISMS CAPABLE OF PRODUCING POLY(HIBA) FROM FEEDSTOCK

(71) Applicant: INDUSTRIAL MICROBES, INC., Alameda, CA (US)

(72) Inventors: Elizabeth Jane CLARKE, Alameda, CA (US); Derek Lorin GREENFIELD, Alameda, CA (US); Noah Charles HELMAN, Alameda, CA (US); Timothy Brian ROTH, Alameda, CA (US); Nyaradzo DZVOVA, Alameda, CA (US)

(73) Assignee: INDUSTRIAL MICROBES, INC., Alameda, CA (US)

(21) Appl. No.: 18/035,889

(22) PCT Filed: Nov. 10, 2021
(86) PCT No.: PCT/US2021/058728

§ 371 Date: May 8, 2023

Related U.S. Application Data
(60) Provisional application No. 63/112,093, filed on Nov. 10, 2020.
(60) Provisional application No. 63/176,027, filed on Apr. 16, 2021.

(51) Int. Cl. C12P 7/42; C12N 9/00; C12N 9/10; C12N 9/02; C12N 9/04; C12N 9/90; C12N 15/70; C12P 7/625

(52) U.S. Cl. CPC C12P 7/42; C12N 9/93; C12N 9/1029; C12N 9/0073; C12N 9/0006; C12N 9/0008; C12N 9/90; C12N 15/70; C12P 7/625; C12Y 114/13025; C12Y 101/01001; C12Y 102/0101; C12Y 602/01001; C12Y 504/99002

(57) ABSTRACT

The present disclosure relates to microorganisms capable of producing poly(hydroxyisobutyric acid) (poly(HIBA)) from feedstocks and methods of producing poly(HIBA), methacrylic acid (MAA), and methacrylate esters (MAE) from feedstocks.

### PRIORITY CLAIM

The instant application claims priority to and the benefit of U.S. Provisional Application No. 63/112,093, filed on Nov. 10, 2020, entitled “Production of Methacrylic Acid and Methacrylate Esters” and U.S. Provisional Application No. 63/176,027, filed on Apr. 16, 2021, entitled “Production of Methacrylic Acid and Methacrylate Esters”, the entire contents of which are incorporated by reference herein and relied upon.

### SEQUENCE LISTING

The instant application contains a Sequence Listing which has been submitted electronically in ASCII format and is hereby incorporated by reference in its entirety. Said ASCII copy, created on Nov. 5, 2021, is named 1107367_00025 SL.txt and is 226,241 bytes in size.

### FIELD

The present disclosure relates to microorganisms capable of producing poly(hydroxyisobutyric acid) (poly(HIBA)) from feedstocks and methods of producing poly(HIBA), methacrylic acid (MAA), and methacrylate esters (MAE) from feedstocks.

### BACKGROUND

Methacrylic acid (MAA) and methacrylate esters (MAE) are useful chemicals that are produced at large scale. More than a million tons of methacrylic acid and methacrylate esters are produced every year. These chemicals find use in common applications such as plastic acrylic glass as a lightweight replacement for glass.

Several methods exist for making MAA and MAE (See, for example, Mahboub et al., “Catalysis for the synthesis of methacrylic acid and methyl methacrylate”, Chem. Soc. Rev., Issue 20, 2018, DOI: 10.1039/c8cs00117k, which is incorporated by reference in its entirety, herein). Unfortunately, these methods result in significant emissions of greenhouse gases.

Previous research has demonstrated that a polymer of 3-hydroxypropionic acid can be converted into acrylic acid using a thermolytic process, followed by a distillation to capture the acrylic acid vapors and then condenses them at a high purity (See, for example, WO 2013/185009, which is incorporated by reference in its entirety, herein). Other researchers have investigated using fermentation of engineered microorganisms for the biological production of 2-hydroxyisobutyric acid (2-HIBA) and 3-hydroxyisobutyric acid (3-HIBA) as potential precursors to MAA (See, for example, U.S. Pat. No. 8,241,877, which is incorporated by reference in its entirety herein).

However, no one has yet succeeded in developing a commercially viable method for biologically producing MAA or 2-HIBA or 3-HIBA. This is likely due to the toxicity of MAA to the microorganism, the high cost associated with purification of product from the fermentation process, the high cost associated with the feedstock (e.g., sugar), and the efficiency of conversion of the feedstock into product.

### SUMMARY

A commercially viable method for producing MAA or poly(2-HIBA) or poly(3-HIBA) is provided herein in the form of engineered microorganisms.

A first aspect is drawn to an engineered microorganism, comprising or consisting of a CoA-ligase and a PHA polymerase, capable of producing a poly(HIBA) from a feedstock. In some embodiments, the poly(HIBA) comprises or consists of poly(2-HIBA) and/or poly(3-HIBA). In some embodiments, the CoA-ligase comprises or consists of one or more of isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: NMar 1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the PHA synthase comprises or consists of one or more of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the CoA-ligase comprises or consists of one or more of polypeptides having at least about 70%, at least about 75%, at least about 80%, at least about 85%, at least about 90%, at least about 95%, at least about 98%, or at least about 99% sequence identity to at least one of Isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar 1309 from Narosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the PHA synthase comprises or consists of one or more of polypeptides having at least about 70%, at least about 75%, at least about 80%, at least about 85%, at least about 90%, at least about 95%, at least about 98%, or at least about 99% sequence identity to at least one of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the feedstock comprises or consists of methane, ethane, propane, methanol, ethanol, propanol, glycerol, glucose, fatty acids, succinic acid, and combinations thereof. In some embodiments, the HIBA comprises or consists of 2-hydroxyisobutyric acid (2-HIBA) and/or 3-hydroxyisobutyric acid (3-HIBA).

In some embodiments, the engineered microorganism further comprises or consists or an engineered pathway for producing a hydroxyisobutyric acid (HIBA) from the feedstock. In some embodiments, the engineered pathway comprises or consists of MMO, ADH, ACDH, and/or acetyl-CoA synthase. In some embodiments, the engineered pathway further comprises or consists of a sleeping beauty mutase (Sbm). In some embodiments, the engineered pathway further comprises or consists of a methylmalonyl-CoA reductase (mmcr or mcr). In some embodiments, the engineered pathway comprises or consists of modifying one or more endogenous enzymes.

In some embodiments, the engineered microorganism is Escherichia coli.

A second aspect provides a method for producing a poly(hydroxyisobutyric acid) (poly(HIBA)) from a feedstock, the method comprising or consisting of: 1) providing a nutrient medium comprising the feedstock; and 2) culturing an engineered microorganism in the nutrient medium, the engineered microorganism comprising or consisting of a CoA-ligase and a polyhydroxyalkanoate (PHA) polymerase. In some embodiments, the poly(HIBA) comprises or consists of poly(2-hydroxyisobutyric acid) (poly(2-HIBA)) and/or poly(3-hydroxyisobutyric acid) (poly(3-HIBA)). In some embodiments, the CoA-ligase comprises or consists of one or more of isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar 1309 from Narosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the PHA synthase comprises or consists of one or more of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the CoA-ligase comprises or consists of one or more of polypeptides having at least about 70%, at least about 75%, at least about 80%, at least about 85%, at least about 90%, at least about 95%, at least about 98%, or at least about 99% sequence identity to at least one of Isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar 1309 from Narosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the PHA synthase comprises or consists of one or more of polypeptides having at least about 70%, at least about 75%, at least about 80%, at least about 85%, at least about 90%, at least about 95%, at least about 98%, or at least about 99% sequence identity to at least one of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the feedstock comprises or consists of methane, ethane, propane, methanol, ethanol, propanol, glycerol, glucose, fatty acids, succinic acid and combinations thereof. In some embodiments, the HIBA comprises or consists of 2-hydroxyisobutyric acid (2-HIBA) and/or 3-hydroxyisobutyric acid (3-HIBA).

In some embodiments, the engineered microorganism further comprises or consists of an engineered pathway for producing a hydroxyisobutyric acid (HIBA) from the feedstock. In some embodiments, the engineered pathway comprises or consists of MMO, ADH, ACDH, and/or acetyl-CoA synthase. In some embodiments, the engineered pathway further comprises or consists of a sleeping beauty mutase (Sbm). In some embodiments, the engineered pathway further comprises or consists of a methylmalonyl-CoA reductase (mmcr). In some embodiments, the engineered pathway comprises or consists of modifying one or more endogenous enzymes.

In some embodiments, the engineered microorganism is Escherichia coli.

In some embodiments, the method further comprises or consists of (i) separating the microorganism from the nutrient medium; and (ii) optionally extracting the poly(HIBA) from the microorganism; and (iii) heating the poly(HIBA) to a temperature in a range from about 150° C. to about 450° C. for a time period from about 0.5 to 120 minutes to produce methacrylic acid (MAA). In some embodiments, the method further comprises or consists of esterifying the MAA with an alcohol to produce a methacrylate ester (MAE). In some embodiments, the method further comprises or consists of separating the poly(HIBA) from the nutrient medium; depolymerizing the poly(HIBA) to HIBA; and converting the HIBA using a catalyst to produce a methacrylic acid (MAA).

A third aspect of the present disclosure provides an nucleic acid construct, comprising or consisting of one or more first polynucleotides encoding a CoA-ligase and a polyhydroxyalkanoate (PHA) polymerase in a first engineered pathway that catalyze a conversion of a feedstock to a poly(HIBA). In some embodiments, the poly(HIBA) comprises or consists of poly(2-HIBA) and/or poly(3-HIBA). In some embodiments, the CoA-ligase comprises or consists of one or more of Isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar_1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the PHA synthase comprises or consists of one or more of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the feedstock comprises or consists of methane, ethane, propane, methanol, ethanol, propanol, glycerol, glucose, fatty acids, succinic acid and combinations thereof. In some embodiments, the HIBA comprises or consists of 2-hydroxyisobutyric acid (2-HIBA) and/or 3-hydroxyisobutyric acid (3-HIBA).

In some embodiments, the nucleic acid construct further comprises or consists of one or more second elements encoding enzymes in an engineered HIBA pathway (or engineered HIBA pathway enzymes) that catalyzes a conversion of the feedstock to a hydroxyisobutyric acid (HIBA). In some embodiments, the one or more second elements are part of or the same as the one or more first polynucleotides. In some embodiments, the one or more second elements are one or more second polynucleotides different from the one or more first polynucleotides.

In some embodiments, the polynucleotides comprise or consist of one or more modifications. In some embodiments, the one or more modifications comprise or consist of polynucleotides encoding, and capable of expressing, one or more chaperone proteins. In some embodiment, the one or more chaperones comprises or consists of groEL and/or groES.

In some embodiments, the engineered HIBA pathway comprises or consists of MMO, ADH, ACDH, and/or acetyl-CoA synthase. In some embodiments, the engineered HIBA pathway further comprises or consists of a sleeping beauty mutase (Sbm). In some embodiments, the engineered HIBA pathway further comprises or consists of a methylmalonyl-CoA reductase (mmcr). In some embodiments, the engineered pathway comprises or consists of modifying one or more endogenous enzymes.

In some embodiments, the protein is expressed in an engineered microorganism in a sufficient amount. In some embodiments, the engineered microorganism is Escherichia coli.

In some embodiments, the CoA-ligase comprises or consists of one or more of polypeptides having at least about 70%, at least about 75%, at least about 80%, at least about 85%, at least about 90%, at least about 95%, or at least about 99% sequence identity to at least one of Isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar_1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12).

In some embodiments, the PHA synthase comprises or consists of one or more of polypeptides having at least about 70%, at least about 75%, at least about 80%, at least about 85%, at least about 90%, at least about 95%, or at least about 99% sequence identity to at least one of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

### BRIEF DESCRIPTION OF THE DRAWINGS

FIG. 1 shows the results of samples analyzed by pyrolysis gas chromatography-mass spectrometry showing formation of MAA.

FIG. 2 shows the results of samples analyzed by pyrolysis gas chromatography-mass spectrometry showing no formation of MAA from a control strain.

### DETAILED DESCRIPTION

Unless otherwise defined, all terms of art, notations and other scientific terminology used herein are intended to have the meanings commonly understood by those of skill in the art to which this present disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

The term “combinations thereof” includes every possible combination of elements to which the term refers.

As used herein, “ACDH” or “acetaldehyde dehydrogenase” is the enzyme that catalyzes the conversion of acetaldehyde to acetyl-CoA. In an embodiment, acetaldehyde dehydrogenase is an acetaldehyde dehydrogenase in EC 1.2.1.10.

As used herein, “Acs” or “acetyl-CoA synthase” or “ACS” shall refer to a class of proteins, enzymes, and enzyme complexes involved in metabolism of acetate. Acetyl-CoA synthetase (EC 6.2.1.1) is in the ligase class of enzymes that activate acetate to acetyl-CoA in an ATP-dependent manner. In an embodiment, acetyl-CoA synthetase is an acetyl-CoA synthase in EC 6.2.1.1.

As used herein, “ADH” or “alcohol dehydrogenase” or “ADHP” shall refer to an ethanol-active medium-chain alcohol dehydrogenase/acetaldehyde reductase. In an embodiment, alcohol dehydrogenase is an alcohol dehydrogenase in EC 1.1.1.1 or 1.1.1.2 or 1.1.2.8 or 1.1.3.13.

As used herein, the term “biomass” is intended to mean the collection of biological matter, made up of cells, that results from the culturing process of a microorganism under suitable conditions for the growth of that organism in culture. In some cases, the biomass includes simply cells and their contents and in other cases, biomass includes macromolecules, such as proteins, that are secreted into the culture outside the boundary of the cell membrane.

As used herein, the term “carbon source” is intended to mean a raw material input to an industrial process that contains carbon atoms that can be used by the microorganisms in a culture.

As used herein, the terms “chaperone,” “protein folding chaperone,” and “folding chaperone” are intended to mean one or more proteins that improve the folding of polypeptide (amino acid) chains into 3-dimensional structures. Protein folding chaperones help their substrates, namely other proteins, to become properly folded and often more highly soluble. Since most proteins must be folded in a particular shape to be functional, the expression of protein folding chaperones can assist in the proper assembly of certain enzymes in a cell and thereby can result in an increase in the enzymatic activity of the substrate proteins.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes and acts in acetyl or other acyl group transfer and in fatty acid synthesis and oxidation, pyruvate oxidation, and in other acetylation.

As used herein, “CoA-ligase” shall refer to a class of proteins, enzymes, and enzyme complexes involved in covalently linking a CoA to another metabolite, such as those designated under EC 6.2.1. In an embodiment, the CoA-ligase (e.g. an enzyme that falls under EC 6.2.1) activity contributes to the conversion of HIBA (including 2-HIBA and 3-HIBA) to HIBA-CoA (including 2-HIBA-CoA and 3-HIBA-CoA). A list of example CoA-ligase enzymes is shown in Table 1.

As used herein, the term “culturing” is intended to mean the growth or maintenance of a microorganism under laboratory or industrial conditions. The culturing of microorganisms is a standard practice in the field of microbiology.

As used herein, “engineered,” “modification,” “genetic alteration,” “genetically altered,” “genetic engineering,” “genetically engineered,” “genetic modification,” “genetically modified,” “genetic regulation,” or “genetically regulated” shall be used interchangeably and refer to direct or indirect manipulation of an organism's genome or genes to produce, for example, a desired effect, such as a desired phenotype.

As used herein, the term “enzyme” shall refer to molecules or biological catalysts that accelerate, or catalyze, chemical reactions. Like all catalysts, enzymes increase the rate of reaction by lowering the activation energy. Almost all metabolic processes in the cell need enzymes in order to occur at rates fast enough to sustain life.

As used herein, “enzymes engineered in a HIBA pathway” or “engineered HIBA pathway enzymes” shall mean a set of enzymes that catalyze the conversion of a feedstock or substrate chemical(s) into product chemical HIBA including 2-HIBA and/or 3-HIBA using one or more enzymatic steps. Enzymes engineered in a HIBA pathway are intended to be, herein, without limitation, the set of enzymes that comprises or consists of one or more of MMO, ADH, ACDH, Sbm, and mmcr. In some embodiments, the engineered pathway comprises or consists of modifying one or more endogenous enzymes.

As used herein, “ethane” shall refer to an organic chemical compound with chemical formula C₂H₆.

As used herein, “ethanol” or “ethyl alcohol” or “grain alcohol” or “drinking alcohol” or “alcohol” or “EtOH” shall refer to an organic chemical compound. It is a simple alcohol with the chemical formula C₂H₆O.

As used herein, “feedstock” or “carbon source” shall refer to a raw material input to an industrial process that contains carbon atoms that can be used by the microorganisms in a culture.

As used herein, “HCL” shall refer to a class of CoA-ligase enzymes in EC 6.2.1 that are involved in covalently linking a CoA to another metabolite. The HCL activity contributes to the conversion of HIBA (including 2-HIBA and 3-HIBA) to HIBA-CoA (including 2-HIBA-CoA and 3-HIBA-CoA).

As used herein, “hydroxyisobutyric acid” or “HIBA” shall refer to a group of four-carbon organic compounds that have both hydroxyl and carboxylic acid functional groups with a chemical formula C₄H₈O₃. There are two isomers, distinguished by the distance between the two functional groups: 2-hydroxyisobutyric acid, also known as 2-methyllactic acid, 2-hydroxy-2-methylpropanoic acid, acetonic acid, alpha-hydroxyisobutyric acid, α-hydroxyisobutyric acid, or 2-HIBA; and 3-hydroxyisobutyric acid, also known as 3-hydroxy-2-methylpropanoic acid, β-hydroxyisobutyric acid, beta-hydroxyisobutyric acid, or 3-HIBA.

As used herein, “isobutyrate-CoA synthetase” or “ICS” shall refer to a class of proteins, enzymes, and enzyme complexes that are involved in covalently linking a CoA to another metabolite. The isobutyrate-CoA synthetase (EC 6.2.1) refers to enzymes that catalyze the conversion of HIBA (including 2-HIBA and 3-HIBA) to HIBA-CoA (including 2-HIBA-CoA and 3-HIBA-CoA).

As used herein, “isocaprenoyl-CoA:2-hydroxyisocaproate CoA-transferase” or “hadA” shall refer to a class of enzymes that have CoA-transferase activity and are involved in covalently linking a CoA to another metabolite. The isocaprenoyl-CoA:2-hydroxyisocaproate CoA-transferase (hadA) (EC 6.2.1) enzyme catalyzes the conversion of HIBA (including 2-HIBA and 3-HIBA) to HIBA-CoA (including 2-HIBA-CoA and 3-HIBA-CoA).

As used herein, “methacrylic acid” or “MAA” shall refer to a compound having the chemical formula CH₂═C(CH₃)CO₂ (IUPAC name 2-methyl-2-propenoic acid) and is the acid form of methacrylate. It is understood that methacrylic acid and methacrylate can be used interchangeably throughout to refer to the compound in any of its neutral or ionized forms, including any salt forms thereof. Those skilled understand that the specific form will depend on the pH.

As used herein, “methacrylate ester” or “MAE” refers to a compound having the chemical formula CH₂═C(CH₃)COOR, wherein R is a lower alkyl, that is C1 to C6, branched or straight chain, including, without limitation, methyl, ethyl, n-propyl, n-butyl, i-propyl, sec-butyl, and tert-butyl, pentyl, or hexyl, any of which can be unsaturation thereby being, for example, propenyl, butenyl, pentyl, and hexenyl. Exemplary methacrylate esters include, without limitation, methyl methacrylate, ethyl methacrylate, and n-propyl methacrylate. Methacrylate esters as used herein also include other R groups that are medium to long chain groups, that is C7-C22, wherein the methacrylate esters are derived from fatty alcohols, such as 2-ethylhexyl, heptyl, octyl, nonyl, decyl, undecyl, lauryl, tridecyl, myristyl, pentadecyl, cetyl, palmitolyl, heptadecyl, stearyl, nonadecyl, arachidyl, heneicosyl, and behenyl alcohols, any one of which can be optionally branched and/or contain unsaturations.

As used herein, “methane” shall refer to a chemical compound with the chemical formula CH₄ (one atom of carbon and four atoms of hydrogen).

As used herein, “methanol” or “methyl alcohol” or “methyl hydrate” shall refer to is a chemical and the simplest alcohol, with the formula CH₃OH (a methyl group linked to a hydroxyl group, often abbreviated MeOH).

As used herein, “methylmalonyl-CoA” shall refer to the thioester consisting of coenzyme A linked to methylmalonic acid. It is an important intermediate in the biosynthesis of many organic compounds as well as in the process of carbon assimilation.

As used herein, “methylmalonyl-CoA reductase” or “mmcr” or “mcr” shall refer to a class of enzymes in EC 1.2.1 that catalyze the cleavage and reduction of methylmalonyl-CoA to produce 3-HIBA.

As used herein, “microbe,” “microbial,” “microbial organism,” or “microorganism” are intended to mean any organism that exists as a microscopic cell that is included within the domains of archaea, bacteria, or eukarya. Microbe is intended to encompass prokaryotic or eukaryotic cells or organisms having a microscopic size and includes bacteria, archaea, and eubacteria of all species as well as eukaryotic microorganisms such as yeast and fungi. The term also includes cell cultures of any species that can be cultured for the production of a product.

As used herein, “MMO” or “methane monooxygenase” shall refer to a class of proteins, enzymes, and enzyme complexes that are capable of oxidizing the C—H bond in methane as well as other alkanes. MMOs include soluble methane monooxygenase (EC 1.14.13.25) and particulate methane monooxygenase (EC 1.14.18.3). Soluble methane monooxygenase belongs to the class of oxidoreductase enzymes (EC 1.14.13.25). MMO activity contributes to the conversion of methane to methanol and ethane to ethanol in an engineered strain of microorganism, such as, for example, without limitation, Escherichia coli.

As used herein, “naturally occurring” shall refer to microorganisms or cultures normally found in nature.

As used herein, “NMar_1309” or “3-hydroxypropionate-CoA ligase” or “3-hydroxypropionyl-CoA synthase” shall refer to an enzyme in EC 6.2.1 or EC 6.2.1.36 that is involved in the hydroxypropionate/hydroxybutyrate (HP/HB) cycle, a modified version of the autotrophic HP/HB cycle of Crenarchaeota. NMar_1309 (EC 6.2.1.36) is involved in covalently linking a CoA to another metabolite. NMar_1309 (EC 6.2.1.36) activity contributes to the conversion of 2-HIBA to 2-HIBA-CoA and the conversion of 3-HIBA to 3-HIBA-CoA.

As used herein, “nucleic acid” or “nucleic acids” shall refer to biopolymers, or large biomolecules, essential to all known forms of life. They are composed of nucleotides, which are the monomers made of three components: a 5-carbon sugar, a phosphate group and a nitrogenous base. The two main classes of nucleic acids are deoxyribonucleic acid (DNA) and ribonucleic acid (RNA).

As used herein, the term “pathway” is intended to mean a set of enzymes that catalyze the conversion of substrate chemical(s) into product chemical(s) using one or more enzymatic steps. A pathway may be a synthetic pathway (comprised of exogenous enzymes) or a partially synthetic pathway (comprised of both exogenous and endogenous enzymes).

As used herein, “PHA polymerase” or “Poly(3-hydroxyalkanoate) polymerase” or “PHA synthase” or “Polyhydroxyalkanoic acid synthase” shall refer to a class of enzymes and enzyme complexes in EC 2.3.1, EC 2.3.1.B2, 2.3.1.B3, 2.3.1.B4, or 2.3.1.B5 that polymerize different monomers with varying substrate specificity profiles (e.g. variable preferences for the hydroxyl group in the 2-, 3-, 4-position, and for total chain length). PHA polymerase activity contributes to the conversion of 2-HIBA-CoA to poly(2-HIBA) and the conversion of 3-HIBA-CoA to poly(3-HIBA). A list of example PHA synthase enzymes is listed in Tables 2 and 3.

As used herein, “phaC” shall refer to a class of enzymes and enzyme complexes that are involved in PHA biosynthesis and function by polymerizing monomeric hydroxyalkanoate substrates such as polymerizing hydroxyacids to a higher molecular weight PHA product. PHA synthase is the key enzyme involved in PHA biosynthesis and functions by polymerizing monomeric hydroxyalkanoate substrates. In an embodiment, phaC is a Poly(3-hydroxyalkanoate) polymerase subunit PhaC (EC 2.3.1). In an embodiment, phaC is phaC from Allochromatium vinosum (SEQ ID NO: 22).

As used herein, “PhaC-PhaE” shall refer to a class of PHA synthases enzymes in EC 2.3.1 that polymerize hydroxyacids to a higher molecular weight PHA product. The PhaC-PhaE polymerizes hydroxyisobutyric Acid-coenzyme A (HIBA-CoA) including 2-HIBA-CoA and/or 3-HIBA-CoA to the high molecular weight PHA product poly-hydroxyisobutyric acid (Poly(HIBA)) including poly(2-HIBA) and/or poly(3-HIBA).

As used herein, “phaC1” shall refer to a class of PHA synthases enzymes in EC 2.3.1 that polymerize hydroxyacids to a higher molecular weight PHA product (https://www.nature.com/articles/s41598-017-05509-04). PhaC1 catalyzes the polymerization of hydroxyisobutyric acid-Coenzyme A (HIBA-CoA) including 2-HIBA-CoA and/or 3-HIBA-CoA to the high molecular weight PHA product poly-hydroxyisobutyric acid (Poly(HIBA)) including poly(2-HIBA) and/or poly(3-HIBA).

As used herein, “PhaC1437” shall refer to a quadruple mutant (E130D, S325T, S477G, and Q481K) of the PhaC enzyme in EC 2.3.1 that polymerizes hydroxyacids to a higher molecular weight PHA product (https://onlinelibrary.wiley.com/doi/abs/10.1002/bit.22547). PhaC1437 (EC 2.3.1) catalyzes the polymerization of hydroxyisobutyric Acid-coenzyme A (HIBA-CoA) including 2-HIBA-CoA and/or 3-HIBA-CoA to the high molecular weight PHA product poly-hydroxyisobutyric acid (Poly(HIBA)) including poly(2-HIBA) and/or poly(3-HIBA).

As used herein, “PHA polymerase 3” shall refer to one of the polyhydroxyalkanoate (PHA) polymerase enzymes in EC 2.3.1 that polymerizes hydroxyacids to a higher molecular weight PHA product. PHA polymerase 3 (EC 2.3.1) catalyzes the polymerization of hydroxyisobutyric Acid-coenzyme A (HIBA-CoA) including 2-HIBA-CoA and/or 3-HIBA-CoA to the high molecular weight PHA product poly-hydroxyisobutyric acid (Poly(HIBA)) including poly(2-HIBA) and/or poly(3-HIBA).

As used herein, “poly(HIBA)” or “poly(hydroxyisobutyric acid)” shall refer to a polymer of hydroxyisobutyric acid (HIBA). Poly(HIBA) includes poly(2-hydroxyisobutyric acid) (poly(2-HIBA)), poly(3-hydroxyisobutyric acid) (poly(3-HIBA)) or any copolymer or mixture thereof.

As used herein, “2-HIBA” or “poly(2-HIBA)” or “poly(2-hydroxyisobutyric acid)” or “poly(2-Hydroxy-2-methylpropanoic acid)” shall refer to a polymer of 2-hydroxyisobutyric acid (2-HIBA) with a chemical formula: H—[—O—CH(CH₃)₂CO-]ₙ—OH

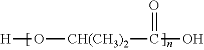

As used herein, “3-HIBA” or “poly(3-HIBA)” or “poly(3-hydroxyisobutyric acid)” or “poly(3-Hydroxy-2-methylpropanoic acid)” shall refer to a polymer of 3-hydroxyisobutyric acid (3-HIBA) with a chemical formula: H—[-O—CH₂CH(CH₃)CO-]ₙ—OH

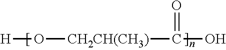

As used herein, “polynucleotide,” “oligonucleotide,” “nucleotide sequence,” and “nucleic acid sequence” are intended to mean one or more polymers of nucleic acids and include, but are not limited to, coding regions, which are transcribed or translated into a polypeptide or chaperone, appropriate regulatory or control sequences, controlling sequences, e.g., translational start and stop codons, promoter sequences, ribosome binding sites, polyadenylation signals, transcription factor binding sites, termination sequences, regulatory domains and enhancers, among others. A polynucleotide, as used herein, need not include all of its relevant or even complete coding regions on a single polymer and the invention provided herein contemplates having complete or partial coding region on different polymers.

As used herein, “propane” shall refer to a three-carbon alkane with the molecular formula C₃H₈.

As used herein, “propanol” shall refer to a primary alcohol with the formula C₃H₇O and sometimes represented as PrOH or n-PrOH.

As used herein, “peptide” shall refer to short chains of amino acids linked by peptide bonds. Chains of fewer than ten or fifteen amino acids are called oligopeptides and include dipeptides, tripeptides, and tetrapeptides.

As used herein, “polypeptide” shall refer to a longer, continuous, unbranched peptide chain. A polypeptide that contains more than approximately fifty amino acids is known as a “protein.” “Proteins” consist of one or more polypeptides arranged in a biologically functional way, often bound to ligands such as coenzymes and cofactors, or to another protein or other macromolecule such as DNA or RNA, or to complex macromolecular assemblies.

As used herein, “Sbm” or “sleeping beauty mutase” or “scpA” shall refer to a methylmalonyl-CoA mutase enzyme in EC 5.4.99.2 that catalyzes the reversible, stereospecific interconversion of succinyl-CoA to methylmalonyl-CoA.

As used herein, “3HP-CoA synthetase” shall refer to a CoA-ligase enzyme in EC 6.2.1 involved in covalently linking a CoA to another metabolite. 3HP-CoA synthetase (EC 6.2.1) activity contributes to the conversion of HIBA (including 2-HIBA and 3-HIBA) to HIBA-CoA (including 2-HIBA-CoA and 3-HIBA-CoA).

Provided herein are engineered microorganisms and methods for producing poly(HIBA) from a feedstock. The engineered microorganisms can have a CoA-ligase and a PHA polymerase. The engineered microorganisms convert feedstocks into 2-HIBA or 3-HIBA and subsequently generate a polymer from these molecules. The polymer is inert inside the cells; it can be extracted and then converted to MAA or MAE using a thermolysis-distillation downstream process. Alternatively, the polymer can be separated from the cells after lysis and then depolymerized. The monomers can subsequently be converted to MAA or MAE via dehydration using a basic catalyst, which removes the hydroxyl group and creates a carbon-carbon double bond.

The key step is the formation of the polymer of 2-HIBA or 3-HIBA, referred to as poly(HIBA) including poly(2-HIBA) and poly(3-HIBA), as this provides a metabolic sink for the HIBA monomers, avoids any product-related toxicity, and avoids acidification of the fermentation broth. The low-cost feedstocks may comprise methane, ethane, propane, methanol, ethanol, propanol, glycerol, glucose, succinic acid, fatty acids, amino acids, sugars, biomass, and combinations thereof.

A first aspect provides an engineered microorganism, comprising or consisting of a CoA-ligase and a PHA polymerase, capable of producing a poly(HIBA) from a feedstock. In some embodiments, the poly(HIBA) comprises or consists of poly(2-HIBA) and/or poly(3-HIBA). In some embodiments, the CoA-ligase comprises or consists of one or more of isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: NMar_1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the PHA synthase comprises or consists of one or more of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the CoA-ligase comprises or consists of one or more polypeptides having at least about 70%, at least about 75%, at least about 80%, at least about 85%, at least about 90%, at least about 95%, at least about 98%, or at least about 99% sequence identity to at least one of Isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar_1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the PHA synthase comprises or consists of one or more of polypeptides having at least about 70%, at least about 75%, at least about 80%, at least about 85%, at least about 90%, at least about 95%, at least about 98%, or at least about 99% sequence identity to at least one of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the engineered microorganism further comprises or consists or an engineered pathway for producing a hydroxyisobutyric acid (HIBA) from the feedstock. In some embodiments, the feedstock comprises or consists of methane, ethane, propane, methanol, ethanol, propanol, and/or combinations thereof. In some embodiments, the HIBA comprises or consists of 2-hydroxyisobutyric acid (2-HIBA) and/or 3-hydroxyisobutyric acid (3-HIBA).

In some embodiments, the engineered pathway comprises or consists of MMO, ADH, ACDH, and/or acetyl-CoA synthase. In some embodiments, the engineered pathway further comprises or consists of a sleeping beauty mutase (Sbm). In some embodiments, the engineered pathway further comprises or consists of a methylmalonyl-CoA reductase (mmcr). In some embodiments, the engineered pathway comprises or consists of modifying one or more endogenous enzymes.

In some embodiments, the engineered microorganism is Escherichia coli.

### Microorganisms

Engineered microorganisms may be derived from any microbe such as, for example, archaea, bacteria, or eukarya, as known to one skilled in the art. In some embodiments, the engineered microorganisms is derived from at least one of Escherichia coli, Bacillus subtilis, Bacillus methanolicus, Pseudomonas putida, Saccharomyces cerevisiae, Pichia pastoris, Pichia methanolica, Salmonella enterica, Corynebacterium glutamicum, Klebsiella oxytoca, Anaerobiospirillum succiniciproducens, Actinobacillus succinogenes, Mannheimia succiniciproducens, Rhizobium etli, Gluconobacter oxydans, Zymomonas mobilis, Lactococcus lactis, Lactobacillus plantarum, Streptomyces coelicolor, Clostridium acetobutylicum, Pseudomonas fluorescens, Pseudomonas chlororaphis, Schizosaccharomyces pombe, Kluyveromyces lactis, Kluyveromyces marxianus, Aspergillus terreus, Aspergillus niger, Yarrowia hpolytica, Hansenula polymorpha, Issatchenkia orientalis, Candida sonorensis, Candida methanosorbosa, Clostridium difficile, Nitrosopumilus maritimus, Sulfolobus solfataricus, Metallosphaera sedula, Allochromatium vinosum, Chromobacterium, Rhodococcus opacus, Betaproteobacterium, and Candida utilis. In some embodiments, the engineered microorganism is derived from Escherichia coli.

In some embodiments, the engineered microorganism comprises or consists of one microorganism. In some embodiments, the engineered microorganisms comprise or consist of one or more microorganisms. In some embodiments, for example, without limitation, one or more engineered microorganisms comprise or consist of CoA-ligase and one or more engineered microorganisms comprise or consist of a PHA polymerase. In some embodiments, the single microorganism or one or more microorganisms comprises or consists of Escherichia coli.

### Feedstock

The feedstock may be a carbon source or any raw material input to an industrial process that contains carbon atoms that can be used by microorganisms in a culture. For example, industrial cultures of microorganisms may use glucose as a source of carbon atoms. In some embodiments, a culture is grown in a medium containing a single usable compound that contains carbon atoms. As carbon is an element that is essential for life, the culture must have metabolic pathways for converting the single compound containing carbon atoms into many other biological molecules necessary for the organism's survival.

Industrial cultures of microorganisms may use glucose as a source of carbon atoms. In addition to typical feedstock such as sugars and amino acids, the carbon source can additionally or also be methane, methanol, ethane, ethanol, propane, propanol, glycerol, glucose, succinic acid, sugars, amino acids, biomass, or any combination of those compounds. A “secondary feedstock” may be used, which refers to a waste material which has been recycled and injected back into use as productive material.

In some embodiments, the feedstock is methane. Methane is a chemical compound with the chemical formula CH₄ (one atom of carbon and four atoms of hydrogen). Methane is the simplest alkane and the main constituent of natural gas. The relative abundance of methane on earth makes it an economically attractive fuel, although capturing and storing it poses technical challenges due to its gaseous state under normal conditions for temperature and pressure.

In some embodiments, the feedstock is methanol. Methanol is the simplest alcohol, with the formula CH₃OH (a methyl group linked to a hydroxyl group, often abbreviated MeOH). Methanol is a light, volatile, colourless, flammable liquid with a distinctive alcoholic odor similar to that of ethanol (potable alcohol). A polar solvent, methanol acquired the name wood alcohol because it was once produced chiefly by the destructive distillation of wood. Today, methanol is mainly produced industrially by hydrogenation of carbon monoxide. Methanol consists of a methyl group linked to a polar hydroxyl group.

In some embodiments, the feedstock is ethane. Ethane is an organic chemical compound with chemical formula C₂H₆. At standard temperature and pressure, ethane is a colorless, odorless gas. Like many hydrocarbons, ethane is isolated on an industrial scale from natural gas and as a petrochemical by-product of petroleum refining. Its chief use is as feedstock for ethylene production.

In some embodiments, the feedstock is ethanol. It is a simple alcohol with the chemical formula C₂H₆O. Its formula can be also written as CH₃—CH₂— OH or C₂H₅OH (an ethyl group linked to a hydroxyl group), and is often abbreviated as EtOH. Ethanol is a volatile, flammable, colorless liquid with a characteristic wine-like odor and pungent taste.

In some embodiments, the feedstock is propane. Propane is a three-carbon alkane with the molecular formula C₃H₈. Propane is a gas at standard temperature and pressure but compressible to a transportable liquid. Propane is a by-product of natural gas processing and petroleum refining. It is commonly used as a fuel in domestic and industrial applications and in low-emissions public transportation.

In some embodiments, the feedstock is propanol. Propanol is a primary alcohol with the formula C₃H₇O and sometimes represented as PrOH or n-PrOH. Propanol is a colorless liquid and has two isomers: 1-propanol with a chemical formula CH₃CH₂CH₂OH; and 2-propanol with a chemical formula CH₃CH(OH)CH₃. It is formed naturally in small amounts during many fermentation processes and used as a solvent in the pharmaceutical industry, mainly for resins and cellulose esters and sometimes as a disinfecting agent.

### HIBA

“HIBA” are four-carbon organic compounds that have both hydroxyl and carboxylic acid functional groups with a chemical formula C₄H₈O₃. There are two isomers, distinguished by the distance between the two functional groups: 2-hydroxyisobutyric acid, also known as 2-methyllactic acid, 2-hydroxy-2-methylpropanoic acid, acetonic acid, alpha-hydroxyisobutyric acid, α-hydroxyisobutyric acid, or 2-HIBA; and 3-hydroxyisobutyric acid, also known as 3-hydroxy-2-methylpropanoic acid, O-hydroxyisobutyric acid, beta-hydroxyisobutyric acid, or 3-HIBA.

In some embodiments, HIBA includes 2-HIBA, 3-HIBA, or a mixture thereof 2-hydroxyisobutyric acid or 2-HIBA is a hydroxyisobutyric acid with the hydroxyl group on the carbon adjacent to the carboxyl with a chemical formula (CH₃)₂CH(OH)COOH. 3-hydroxyisobutyric acid or 3-HIBA is an organic compound with a chemical formula CH₂(OH)CH(CH₃)COOH. Poly(2-HIBA) is a polymer of 2-hydroxyisobutyric acid (2-HIBA) with a chemical formula: H—[—O—CH(CH₃)₂CO-]ₙ—OH. Poly(3-HIBA) is a polymer of 3-hydroxyisobutyric acid (3-HIBA) with a chemical formula: H—[-O—CH₂CH(CH₃)CO-]ₙ—OH.

### MAA or MAE

MAA is a compound that has the chemical formula CH₂═C(CH₃)CO₂ (IUPAC name 2-methyl-2-propenoic acid) and is the acid form of methacrylate. MAA is a colorless, viscous liquid that is a carboxylic acid with an acrid unpleasant odor. MAA is soluble in warm water and miscible with most organic solvents. Methacrylic acid is produced industrially on a large scale as a precursor to its esters. MAA occurs naturally in small amounts in the oil of Roman chamomile.

MAE is a compound that has the chemical formula CH₂═C(CH₃)COOR, wherein R is an alkyl, branched or straight chain, including, without limitation, methyl, ethyl, n-propyl, n-butyl, i-propyl, sec-butyl, tert-butyl, pentyl, hexyl, heptyl, octyl, nonyl, decyl, undecyl, lauryl, tridecyl, myristyl, pentadecyl, cetyl, palmitolyl, heptadecyl, stearyl, nonadecyl, arachidyl, heneicosyl, and behenyl alcohols, any one of which can be optionally branched and/or contain unsaturations.

Pathway from HIBA to HIBA-CoA and Subsequently to Poly(HIBA)

Conversion of HIBA to poly(HIBA) requires two enzymatic steps as shown below in Reactions (1) and (2) for conversion of 2-HIBA and 3-HIBA respectively. As shown in reactions (1) and (2), HIBA is first converted to HIBA-CoA using a CoA-ligase (EC 6.2.1). Second, the HIBA-CoA is then polymerized using a polyhydroxyalkanoate (PHA) polymerase (such as those in EC 2.3.1.B2; 2.3.1.B3; 2.3.1.B4; or 2.3.1.B5) to form poly(HIBA).

2-HIBA→2-HIBA-CoA→poly(2-HIBA) (1)

3-HIBA→3-HIBA-CoA→poly(3-HIBA) (2)

### CoA-Ligase Enzymes

The engineered microorganism has at least one CoA-ligase. Many enzymes are capable of performing the CoA-ligation step (EC 6.2.1). These enzymes often have promiscuous activity against many substrates, although some may have higher activity against 2-HIBA vs. 3-HIBA, or vice versa.

In some embodiments, the at least one CoA-ligase comprises or consists of one or more enzymes capable of performing the CoA-ligation step by catalyzing the conversion of HIBA (including 2-HIBA and 3-HIBA) to HIBA-CoA (including 2-HIBA-CoA and 3-HIBA-CoA). In some embodiments, the at least one CoA-ligase comprise or consist of one or more enzymes from Table 1.

TABLE 1
A list of example CoA-ligases with the gene names and the source organisms.

| Gene Name | Source Organism | Reference |
|---|---|---|
| acs | Escherichia coli | https://ecocyc.org/gene?orgid=ECOLI&id=EG11448 |
| fadK | Escherichia coli | https://ecocyc.org/gene?orgid=ECOLI&id=EG12357 |
| prpE | Escherichia coli | https://ecocyc.org/gene?orgid=ECOLI&id=G6200 |
| AAE11 | Arabidopsis thaliana | https://www.uniprot.org/uniprot/Q9C8D4 |
| pcs | Chloroflexus | Birgit E Alber, et al., “Propionyl-coenzyme A |
| | aurantiacus | synthase from Chloroflexus aurantiacus, a key |
| | | enzyme of the 3-hydroxypropionate cycle for |
| | | autotrophic CO2 fixation,” J. Biol. Chem., 2002 Apr. |
| | | 5; 277(14): 12137-43. |
| | | https://pubmed.ncbi.nlm.nih.gov/11821399/ |
| HCL | Aquincola | Michael Zahn, et al., “Structures of |
| | tertiaricarbonus | 2-Hydroxyisobutyric Acid-CoA Ligase Reveal |
| | L108 | Determinants of Substrate Specificity and Describe a |
| | | Multi-Conformational Catalytic Cycle,” J. of |
| | | Molecular Biology, Volume 431, Issue 15, 12 Jul. |
| | | 2019, Pages 2747-2761. |
| | | https://www.sciencedirect.com/science/article/abs/pii/ |
| | | S0022283619303092 |
| lvaE | Pseudomonas putida | Chandran Sathesh-Prabu, et a., “Engineering the lva |
| | | operon and Optimization of Culture Conditions for |
| | | Enhanced Production of 4-Hydroxyvalerate from |
| | | Levulinic Acid in Pseudomonas putida KT2440,” J. |
| | | Agric. Food Chem. 2019, 67, 9, 2540-2546. |
| | | https://pubs.acs.org/doi/10.1021/acs.jafc.8b06884 |
| alkK | Pseudomonas | Van Beilen, et al., “DNA sequence determination and |
| | oleovorans | functional characterization of the OCT-plasmid- |
| | | encoded alkJKL genes of Pseudomonas oleovorans.” |
| | | Mol. Microbiol. 1992 NOV; 6(21): 3121-3136 (192) |
| orfZ | Clostridium kluyveri | https://biocyc.org/gene?orgid=META&id=MONOMER-13467 |
| Pct540 | Clostridium | Choi, S., et al. “One-step fermentative production of |
| | propionicum | poly(lactate-co-glycolate) from carbohydrates in |
| | | Escherichia coli.” Nat. Biotechnol. 34, 435-440 |
| | | (2016). https://doi.org/10.1038/nbt.3485 |
| HadA | Clostridium difficile | Genbank: AAV40822.1 |
| MatB | Rhizobium trifolii | Irina Koryakina, et al., “Poly Specific trans- |
| | | Acyltransferase Machinery Revealed via Engineered |
| | | Acyl-CoA Synthetases”, ACS Chem. Biol. 2013, 8, |
| | | 200-208, dx.doi.org/10.1021/cb3003489 |
| MatB | Streptomyces | Genbank: QKN66228.1 |
| | coelicolor | |
| ACS T324G | Arabidopsis thaliana | Naazneen Sofeo, et al., “Altering the Substrate |
| | | Specificity of Acetyl-CoA Synthetase by Rational |
| | | Mutagenesis of the Carboxylate Binding Pocket”, |
| | | ACS Synth. Biol. 2019, 8, 1325-1336, DOI: |
| | | 10.1021/acssynbio.9b00008 |
| ACS W427S | Arabidopsis thaliana | Naazneen Sofeo, et al., “Altering the Substrate |
| | | Specificity of Acetyl-CoA Synthetase by Rational |
| | | Mutagenesis of the Carboxylate Binding Pocket”, |
| | | ACS Synth. Biol. 2019, 8, 1325-1336, DOI: |
| | | 10.1021/acssynbio.9b00008 |
| ICS | Pseudomonas | Naazneen Sofeo, et al., “Altering the Substrate |
| | chlororaphis | Specificity of Acetyl-CoA Synthetase by Rational |
| | | Mutagenesis of the Carboxylate Binding Pocket”, |
| | | ACS Synth. Biol. 2019, 8, 1325-1336, DOI: |
| | | 10.1021/acssynbio.9b00008 |
| 3HP-CoA | Metallosphaera | Birgit E. Alber, et al., “3-Hydroxypropionyl- |
| synthetase | sedula | Coenzyme A Synthetase from Metallosphaera sedula, |
| (Modified ACS) | | an Enzyme Involved in Autotrophic CO2 Fixation”, |
| | | Journal of Bacteriology, Febuary 2008, p. 1383-1389 |
| 3HP-CoA | Sulfolobus tokodaii | Birgit E. Alber, et al., “3-Hydroxypropionyl- |
| synthetase | | Coenzyme A Synthetase from Metallosphaera sedula, |
| | | an Enzyme Involved in Autotrophic CO2 Fixation”, |
| | | Journal of Bacteriology, Febuary 2008, p. 1383-1389 |
| ACS | Sulfolobus | Birgit E. Alber, et al., “3-Hydroxypropionyl- |
| | solfataricus | Coenzyme A Synthetase from Metallosphaera sedula, |
| | | an Enzyme Involved in Autotrophic CO2 Fixation”, |
| | | Journal of Bacteriology, Febuary 2008, p. 1383-1389 |
| | | GenBank: NP_344510 |
| 3HP-CoA | Nitrosopumilus | Martin Könneke, et al., “Ammonia-oxidizing archaea |
| synthetase | maritimus | use the most energy efficient aerobic pathway for |
| | | CO2 fixation”, Proceedings of the National Academy |
| | | of Sciences PNAS, vol. 111, no. 22, p. 8239-8244, |
| | | www.pnas.org/cgi/doi/10.1073/pnas. 1402028111 |
| 3HP-CoA | Nitrosopumilus | Martin Könneke, et al., “Ammonia-oxidizing archaea |
| synthetase | maritimus (Strain | use the most energy efficient aerobic pathway for |
| (NMar-1309) | SCM1) | CO2 fixation”, Proceedings of the National Academy |
| | | of Sciences PNAS, vol. 111, no. 22, p. 8239-8244, |
| | | www.pnas.org/cgi/doi/10.1073/pnas. 1402028111 |

In some embodiments, the at least one CoA-ligase comprises or consists of one or more of isocaprenoyl-CoA:2-hydroxyisocaproate CoA-transferase, isobutyrate-CoA synthetase (ICS), NMar_1309, HCL, acs, and/or 3HP-CoA synthetase. In some embodiments, the at least one CoA-ligase comprises or consists of isocaprenoyl-CoA:2-hydroxyisocaproate CoA-transferase (HadA) from Clostridium difficile (SEQ ID NO: 3), isobutyrate-CoA synthetase (ICS) from Pseudomonas chlororaphis (SEQ ID NO: 10), NMar_1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12).

In some embodiments, the at least one CoA-ligase comprises or consists of one or more of polypeptides having at least about 70%, at least about 75%, at least about 80%, at least about 85%, at least about 90%, at least about 95%, or at least about 99% sequence identity to at least one of Isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar_1309 from Nitrosopumilus maritimus SCM1 (SEQ ID NO: 15), HCL from A. tertiaricarbonus L108 (SEQ ID NO: 4), acs from S. sulfaraticus (SEQ ID NO: 14), and/or 3HP-CoA synthetase from Metallosphaera sedula (SEQ ID NO: 12).

In some embodiments, the at least one CoA-ligase comprises or consists of isocaprenoyl-CoA:2-hydroxyisocaproate CoA-transferase. In some embodiments, the isocaprenoyl-CoA:2-hydroxyisocaproate CoA-transferase (HadA) is from Clostridium difficile (SEQ ID NO: 3). Isocaprenoyl-CoA:2-hydroxyisocaproate CoA-transferase catalyzes the conversion of HIBA (including 2-HIBA and 3-HIBA) to HIBA-CoA (including 2-HIBA-CoA and 3-HIBA-CoA).

In some embodiments, the at least one CoA-ligase comprises or consists of one or more of isobutyrate-CoA synthetase (ICS). In some embodiments, the isobutyrate-CoA synthetase (ICS) is from Pseudomonas chlororaphis (SEQ ID NO: 10). Isobutyrate-CoA synthetase catalyzes the conversion of HIBA (including 2-HIBA and 3-HIBA) to HIBA-CoA (including 2-HIBA-CoA and 3-HIBA-CoA).

In some embodiments, the at least one CoA-ligase comprises or consists of NMar_1309. In some embodiments, NMar_1309 is from Nitrosopumilus maritimus SCM1 (SEQ ID NO: 15). NMar_1309 is an enzyme involved in the hydroxypropionate/hydroxybutyrate (HP/HB) cycle, a modified version of the autotrophic HP/HB cycle of Crenarchaeota. Nmar_1309 catalyzes the formation of 3-hydroxypropionyl-CoA, ADP, and phosphate from 3-hydroxypropionate, coenzyme A (CoA) and ATP.

In some embodiments, the at least one CoA-ligase comprises of consists of HCL. In some embodiments, the HCL is from A. tertiaricarbonus L108 (SEQ ID NO: 4). HCL activity contributes to the conversion of HIBA (including 2-HIBA and 3-HIBA) to HIBA-CoA (including 2-HIBA-CoA and 3-HIBA-CoA).

In some embodiments, the at least one CoA-ligase comprises or consists of acs. In some embodiments, the acs is from Sulfolobus solfataricus (SEQ ID NO: 14). Acs is a class of enzymes involved in covalently linking a CoA to another metabolite. Acs is in the ligase class of enzymes that activate acetate to acetyl-CoA in an ATP-dependent manner. Acs activity contributes to the conversion of HIBA (including 2-HIBA and 3-HIBA) to HIBA-CoA (including 2-HIBA-CoA and 3-HIBA-CoA).

In some embodiments, the at least one CoA-ligase comprises or consists of 3HP-CoA synthetase. In some embodiments, the 3HP-CoA synthetase is from Metallosphaera sedula (SEQ ID NO: 12). 3HP-CoA synthetase activity contributes to the conversion of HIBA (including 2-HIBA and 3-HIBA) to HIBA-CoA (including 2-HIBA-CoA and 3-HIBA-CoA).

### PHA Polymerase or PHA Synthase Enzymes

The engineered microorganism has at least one polymerase or PHA synthase. PHA synthases have been categorized into four major classes based on their primary sequences, substrate specificity, and subunit composition. Class I comprise enzymes consisting of only one type of PhaC, which forms a homodimer, while Class II contains two types of synthases, PhaC1 and PhaC2. Class III and IV synthases form heterodimers, comprising PhaC-PhaE and PhaC-PhaR, respectively. Class I, III, and IV synthases tend to favor short-chain-length (SCL) monomers comprising C3-C5 carbon chain lengths.

PHA synthase enzymes have been known to polymerize different monomers with varying substrate specificity profiles (e.g. variable preferences for hydroxyl group in the 2-, 3-, 4-position, and for total chain length). A typical example of a C4 SCL monomer is (R)-3-hydroxybutyrate (3HB), and PhaC polymerizes the acyl moieties of 3-hydroxybutyryl-coenzyme A (3HB-CoA) to the high molecular weight PHA product poly-hydroxybutyrate (PHB). Class II synthases favor medium-chain-length (MCL) monomers comprising C6-C14 carbon chain lengths, such as the C6 monomer 3-hydroxyhexanoate (3HHx).

In some embodiments, the at least one PHA synthase comprises or consists of an enzyme that is capable of performing the polymerization step by catalyzing the conversion of HIBA-CoA to poly(HIBA). In some embodiments, the at least one PHA synthase comprises or consists of one or more enzymes in Table 2 or one or more enzymes in Table 3.

TABLE 2
A list of example PHA-synthase enzymes with gene names and the source organisms.

| | Gene | | |
|---|---|---|---|
| Class | Name | Source Organism | Reference |
| I | phaC | Cupriavidus necator/ | https://www.uniprot.org/uniprot/P23608 |
| | | Ralstonia eutropha | |
| I | phaC1 | Chromobacterium | Min Fey Chek, et al., “Structure of polyhydroxyalkanoate |
| | | USM2 | (PHA) synthase PhaC from Chromobacterium sp. USM2, |
| | | | producing biodegradable plastics,” Sci Rep 7, 5312 |
| | | | (2017). https://doi.org/10.1038/s41598-017-05509-4 |
| | | | https://www.nature.com/articles/s41598-017-05509-4 |
| I | phaC | Aeromonas caviae | https://www.uniprot.org/uniprot/O32471 |
| II | PhaC1437 | Pseudomonas sp. | Taek Ho Yang, et al., “Biosynthesis of polylactic acid and |
| | | MBEL 6-19 | its copolymers using evolved propionate CoA transferase |
| | | | and PHA synthase,” Biotechnology and Bioengineering, |
| | | | Vol. 105, No. 1, Jan. 1, 2010. |
| | | | https://doi.org/10.1002/bit.22547 |
| | | | https://onlinelibrary.wiley.com/doi/abs/10.1002/bit.22547 |
| III | PhaC, PhaE | Allochromatium | W. Yuan, et al, “Class I and III Polyhydroxyalkanoate |
| | | vinosum | Synthases from Ralstonia eutropha and Allochromatium |
| | | | vinosum: Characterization and Substrate Specificity |
| | | | Studies”, Archives of Biochemistry and Biophysics, Vol. |
| | | | 394, No. 1, October 2001. DOI: |
| | | | 10.1006/abbi.2001.2522 |
| III | PhaC, PhaE | Thiocapsa pfennigii | Bernd H A Rehm, “Polyester Synthases: Natural Catalysts |
| | | | for Plastics”, Biochem. J. (2003) 376, 15-33 |
| | | | DOI: 10.1042/BJ20031254 |
| III | PhaC, PhaE | Candidatus | Dong, H., Liffland, et al., “Engineering in Vivo |
| | | Accumulibacter | Production of α-Branched Polyesters”, J. Am. Chem. Soc. |
| | | phophatis clade IIA | 2019, 141, 16877-16883. DOI: 10.1021/jacs.9b08585 |
| | | str. UW-1 | |
| IV | PhaC, | Bacillus megaterium | Takeharu Tsuge, et al., “Class IV polyhydroxyalkanoate |
| | PhaR | | (PHA) synthases and PHA-producing Bacillus,” Applied |
| | | | Microbiology and Biotechnology volume 99, pages 6231- |
| | | | 6240 (2015). |
| | | | https://link.springer.com/article/10.1007/s00253-015- |
| | | | 6777-9 |

TABLE 3
A list of example PHA-synthase enzymes with
gene names and the source organisms.

| Gene | | |
|---|---|---|
| Name | Genus/Species | Gene Bank or Uniprot |
| phaC | Casimicrobium | WP_156862910.1 |
| | huifangae | |
| phaC | Burkholderiales | MBL8309203.1 |
| phaC | Rhizobacter | MBC7708889.1 |
| phaC | Betaproteobacteria | TAG82764.1 |
| phaC | Blastococcus sp. | WP_135138512.1 |
| phaC | Burkholderia cepacia | https://www.uniprot.org/uniprot/G3M4I8 |
| phbC | Rhodococcus ruber | CDZ89462.1 |
| phaC | Rhodococcus opacus | AHK27354.1 |
| | PD630 (1) | |
| phaC | Rhodococcus opacus | AHK28081.1 |
| | PD630 (2) | |
| phaC | Rhodococcus opacus | AHK31697.1 |
| | PD630 (3) | |

In some embodiments, the at least one PHA synthase comprises or consists of one or more of PhaC-PhaE, phaC1, PhaC1437, PHA polymerase 3, and/or phaC. In some embodiments, the at least one PHA synthase comprises or consists of one or more of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the at least one PHA synthase comprises or consists of one or more of polypeptides having at least about 70%, at least about 75%, at least about 80%, at least about 85%, at least about 90%, at least about 95%, at least about 98%, or at least about 99% sequence identity to at least one of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the at least one PHA synthase comprises or consists of one or more phaC. In some embodiments, the one or more phaC is from Betaproteobacterium. PhaC is involved in PHA biosynthesis and function by polymerizing monomeric hydroxyalkanoate substrates. PhaC is the key enzyme involved in PHA biosynthesis and functions by polymerizing monomeric hydroxyalkanoate substrates.

In some embodiments, the at least one PHA synthase comprises or consists of one or more PhaC-PhaE. In some embodiments, the one or more PhaC-PhaE is from Allochromatium vinosum. PhaC-PhaE is a class of PHA synthases that polymerize hydroxyacids to a higher molecular weight PHA product. The PhaC-PhaE catalyze the conversion of hydroxyisobutyric Acid-coenzyme A (HIBA-CoA) including 2-HIBA-CoA and/or 3-HIBA-CoA to poly-hydroxyisobutyric acid (Poly(HIBA)) including poly(2-HIBA) and/or poly(3-HIBA).

In some embodiments, the at least one PHA synthase comprises or consists of one or more phaC1. In some embodiments, the one or more phaC1 is from Chromobacterium USM2. PhaC1 catalyzes the polymerization of hydroxyisobutyric Acid-coenzyme A (HIBA-CoA) including 2-HIBA-CoA and/or 3-HIBA-CoA to poly-hydroxyisobutyric acid (Poly(HIBA)) including poly(2-HIBA) and/or poly(3-HIBA). PhaC1 may favor medium-chain-length (MCL) monomers comprising C6-C14 carbon chain lengths, such as the C6 monomer 3-hydroxyhexanoate (3HHx)

In some embodiments, the at least one PHA synthase comprises or consists of one or more PhaC1437. In some embodiments, the one or more PhaC1437 is from Pseudomonas. PhaC1437 is a quadruple mutant (E130D, S325T, S477G and Q481K) of the PhaC enzyme. PhaC1 catalyzes the polymerization of the hydroxyisobutyric Acid-coenzyme A (HIBA-CoA) including 2-HIBA-CoA and/or 3-HIBA-CoA to poly-hydroxyisobutyric acid (Poly(HIBA)) including poly(2-HIBA) and/or poly (3-HIBA).

In some embodiments, the at least one PHA synthase comprises or consists of one or more PHA polymerase 3. In some embodiments, the one or more PHA polymerase 3 is from Rhodococcus opacus. PHA polymerase 3 catalyzes the polymerization of hydroxyisobutyric Acid-coenzyme A (HIBA-CoA) including 2-HIBA-CoA and/or 3-HIBA-CoA to poly-hydroxyisobutyric acid (Poly(HIBA)) including poly(2-HIBA) and/or poly(3-HIBA).

### Protein Engineering to Identify Improved PHA Synthase Variants

No PHA synthase enzyme has been published previously and shown to have activity against 2-HIBA or 3-HIBA. However, even a small activity of a PHA synthase enzyme can be improved by protein engineering. Directed evolution is a method of improving enzymes that is well known to those skilled in the art. Briefly, the process consists of iterations of three steps: generating genetic diversity, assaying (screening or selecting) the diversity for a property of interest to identify beneficial, neutral, and deleterious mutations, and the recombination of a subset of the mutations which can then be screened for improved mutants. These genetic variants may be used as templates either for additional rounds of recombination of the subset of mutations or for the discovery of additional genetic diversity. Depending on the system of interest, the methods used to generate the genetic diversity, to assay the mutants, and to recombine the mutations may vary.

Many methods are available for the generation of genetic diversity in a DNA sequence: chemical mutagenesis, ultraviolet-light-induced mutagenesis, error-prone PCR, directed saturation mutagenesis, and others. Any combination of these methods may also be used. The goal is to identify mutations that are beneficial, or at worst neutral, for the desired function of the enzyme.

These can then be subject to genetic recombination and screened for improved functionality. Thus, any series of methods of generating genetic diversity that will eventually achieve this goal will be sufficient. An optimal method would connect each mutation to its effect on the enzyme's desired function(s), which may be measured along one or more features or dimensions.

Assaying for PHA in Escherichia coli has been studied by other groups. There are a range of possible methods, including optical scattering, Nile Red fluorescence (“A sensitive, viable-colony staining method using Nile Red for direct screening of bacteria that accumulate polyhydroxyalkanoic acids and other lipid storage compounds,” Archives of Microbiology 171(2):73-80 February 1999, DOI: 10.1007/s002030050681; “High-throughput screen for poly-3-hydroxybutyrate in Escherichia coli and Synechocystis sp. strain PCC6803,”Appl. and Environ. Microbiol., May 2006, p. 3412-3417, DOI: 10.1128/AEM.72.5.3412-3417.2006), biosensors, pyrolysis GCMS, and others.

Once a list of desirable mutations has been generated, one can recombine these mutations in order to test whether the combinations display a desirable activity that exceeds either of the parental sequences. These combinations can be tested either by deliberately constructing specifically desired clones, or by recombining the mutations randomly in a “one-pot” reaction.

Methods of DNA construction for recombination libraries are well-known to those skilled in the art, and include a variety of techniques, including SOE PCR, transfer PCR, and Quikchange mutagenesis (Agilent Technologies). Once the recombined mutants have been constructed, one can assay these variants using the same techniques that were used previously to assay the original mutant libraries or using other assays that measure the enzymes' properties.

### Engineered Pathways

In some embodiments, the engineered microorganism comprises or consists of an engineered pathway. A key factor in developing an economically viable process to high-quality MAA or MAE is to engineer a high-purity PHA from a low-cost feedstock. Although many biological systems can consume carbon sources such as glucose, glycerol, or fatty acids, a particularly useful feedstock is one that is low cost and generates the product at high yield. In the case of MAA and MAE, the feedstocks methane, ethane, and propane are all excellent options, because of their low cost and high yield to MAA and MAE.

If the polymer inside the cell contains a mixture of different monomers, then the resulting product will be a mixture of acids, esters, or other molecules. A mixture is undesirable for the downstream applications of these molecules, but especially in the case where it is difficult or costly to separate these mixtures into pure chemicals using standard chemical engineering methods. One way to avoid this outcome is to use a biological pathway where the biological metabolites only have a single CoA-linked moiety that can be polymerized.

One exemplary pathway from a feedstock ethane to 3-HIBA is shown below in Reactions (3)-(5). In this pathway, ethane is converted to ethanol using methane monooxygenase (MMO). The enzyme ADH converts ethanol into acetaldehyde, which is then turned into acetyl-CoA by the enzyme ACDH or by the enzyme acetyl-CoA synthase (acs) via acetate.

Acetyl-CoA is a major node of central metabolism. It is converted efficiently into succinate using the glyoxylate bypass where two molecules of acetyl-CoA produce one succinate molecule as described at (https://ecocvc.org/ECOLI/NEW-IMAGE?type=PATHWAY&object=TCA-GLYOX-BYPASS&detail-level=2), the entire contents of which are incorporated herein by reference for all purposes and relied upon. This succinate molecule is converted into methylmalonyl-CoA via genes known as sleeping beauty mutase (sbm/scpA) as described at (https://ecocyc.org/gene?orgid=ECOLI&id=EG11444), the entire contents of which are incorporated herein by reference for all purposes and relied upon. The methylmalonyl-CoA is then converted to 3-HIBA using a methylmalonyl-CoA reductase (mmcr) enzyme (for example, from Chloraflexus aurantiacus).

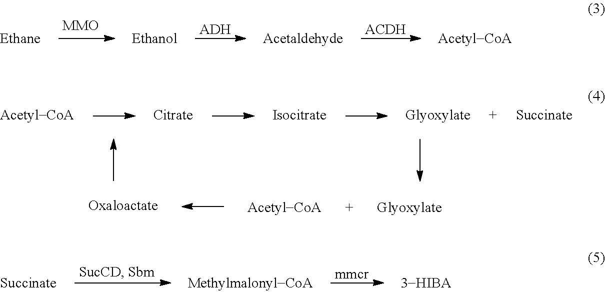

In some embodiments, the engineered pathway comprises or consists of one or more of MMO, ADH, ACDH, and/or acetyl-CoA synthase.

In some embodiments, the engineered pathway comprises or consists of one or more MMOs. MMOs are a class of proteins, enzymes, and enzyme complexes that are capable of oxidizing the C—H bond in methane as well as other alkanes. Methane monooxygenase (MMO) belongs to the class of oxidoreductase enzymes (EC 1.14.13.25). MMO activity contributes to the conversion of ethane to ethanol in an engineered strain of microorganism such as Escherichia coli.

Naturally occurring methane-consuming microorganisms have evolved at least two classes of monooxygenase enzymes: soluble monooxygenases (“sMMO”) and particulate monooxygenases (“pMMO”). Any enzyme or enzyme complex of these categories, any mutated enzyme or complex, or any researcher-designed enzyme or enzyme complex that converts methane into methanol, would be considered a methane monooxygenase enzyme. Many of these enzymes may also oxidize a wide range of substrates, such as ethane into ethanol, and thus act as an ethane monooxygenase.

In some embodiments, the one or more MMOs comprises or consists of one or more sMMOs. The sMMO from Methylococcus capsulatus (Bath) is well-studied. The Methylococcus capsulatus (Bath) can act as a hydroxylate for a large number of substrates (See, Petroleum Biotechnology by Vazquez-Duhalt and Quintero-Romero in 2004, which is incorporated by reference in its entirety herein). The sMMO from Methylococcus capsulatus (Bath) is able to hydroxylate dozens of substrates into an even larger number of products, when assayed in vitro. In some embodiments, the MMO comprises or consists of the monooxygenase from Methylococcus capsulatus (Bath).

In some embodiments, the one or more MMOs comprises or consists of a methane monooxygenase from one or more Methylosinus trichosporium OB3b, Methylomonas methanica, Methylocaldum sp.175, Methyloferula stellata, Methylocystis LW5, Solimonas aquatica (DSM 25927), Methylovulum miyakonense, Rhodococcus ruber IGEM 231, and/or Conexibacter woesei.

In some embodiments, the one or more MMOs comprises or consists of a monooxygenase as set forth in the following Table 4:

| Organism | Gene names | Accession number |
|---|---|---|
| Methylococcus capsulatus | mmoXYBZDC_G | AF525283.1, M90050.3 |
| (Bath) | | |
| Methylosinus trichosporium | mmoXYBZDC, groEL | X55394.3, EF685207.1 |
| OB3b | | |
| Methylococcus capsulatus | pmoCAB | L40804.2 |
| (Bath) | | |
| Methylosinus trichosporium | pmoCAB | U31650.2 |
| OB3b | | |

In some embodiments, the one or more MMOs comprises or consists of one or more pMMOs. This protein complex is composed of three subunits and resides in the inner membrane of the native organism.

In some embodiments, the one or more pMMOs comprises or consists of a pMMO. This protein complex is composed of three subunits and resides in the inner membrane of the native organism. To successfully express the pMMO in Escherichia coli, correct N-terminal leader sequences must be properly fused to each of the three subunits. In some embodiments, the MMO comprises or consists of the pMMO fromMethylococcus capsulatus (See, Elliot, S. et al, Regio- and Stereo selectivity of particulate methane monooxygenase from Methylococus capsulatus (Bath), J. Am. Chem. Soc. 119, 9949-9955 (1997), which is incorporated by reference in its entirety herein).

In some embodiments, the engineered pathway comprises or consists of one or more ACDHs. In some embodiments, the one or more ACDHs is from Escherichia coli or Corynebacterium glutamicum. ACDH catalyzes the conversion of acetaldehyde to acetyl-CoA.

In some embodiments, the engineered pathway comprises or consists of one or more acetyl-CoA synthases. In some embodiments, the one or more acetyl-CoA synthases are from Escherichia coli.

Acetyl-CoA synthases are a class of proteins, enzymes, and enzyme complexes involved in metabolism of acetate. Acetyl-CoA synthase is in the ligase class of enzymes that activate acetate to acetyl-CoA in an ATP-dependent manner. Acetyl-CoA synthase activity constitutes one of two distinct pathways by which Escherichia coli activates acetate to acetyl-CoA. The acetyl-CoA synthase pathway (acetate conversion to acetyl-CoA) functions in a mainly anabolic role, scavenging acetate present in the extracellular medium. Induction of acetyl-CoA synthase expression functions as the metabolic switch activating this pathway.

In some embodiments, the engineered pathway comprises or consists of one or more alcohol dehydrogenases. Alcohol dehydrogenases are a group of dehydrogenase enzymes that occur in many organisms and facilitate the interconversion between alcohols and aldehydes or ketones with the reduction of nicotinamide adenine dinucleotide (NAD⁺) to NADH. In yeast, plants, and many bacteria, some alcohol dehydrogenases catalyze the opposite reaction as part of fermentation to ensure a constant supply of NAD⁺. Alcohol dehydrogenase is more efficient in the reverse direction of acetaldehyde reduction. In some embodiments, ADH activity contributes to the conversion of ethanol to acetaldehyde in an engineered strain of microorganism such as, for example, without limitation, Escherichia coli.

In some embodiments, the engineered pathway comprises or consists of one or more sleeping beauty mutase. Sleeping beauty mutase is a methylmalonyl-CoA mutase enzyme that catalyzes the reversible, stereospecific interconversion of succinyl-CoA to methylmalonyl-CoA.

In some embodiments, the engineered pathway comprises or consists of one or more methylmalonyl-CoA reductase (mcr or mmcr). In some embodiments, the one or more methylmalonyl-CoA reductases is from Chloroflexus aurantiacus. Methylmalonyl-CoA reductase is class of enzymes that catalyze the cleavage and reduction of methylmalonyl-CoA to produce 3-HIBA. In some embodiments, the methylmalonyl-CoA reductase is from Chloroflexus aurantiacus.

### Method of Production

A second aspect provides a method for producing a poly(hydroxyisobutyric acid) (poly(HIBA)) from a feedstock, the method comprising or consisting of: 1) providing a nutrient medium comprising the feedstock and 2) culturing an engineered microorganism in the nutrient medium, the engineered microorganism comprising or consisting of a CoA-ligase and a polyhydroxyalkanoate (PHA) polymerase. In some embodiments, the poly(HIBA) comprises or consists of poly(2-hydroxyisobutyric acid) (poly(2-HIBA)) and/or poly(3-hydroxyisobutyric acid) (poly(3-HIBA)). In some embodiments, the CoA-ligase comprises or consists of one or more of isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar_1309 from Narosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the PHA synthase comprises or consists of one or more of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the CoA-ligase comprises or consists of one or more of polypeptides having at least about 70%, at least about 75%, at least about 80%, at least about 85%, at least about 90%, at least about 95%, at least about 98%, or at least about 99% sequence identity to at least one of Isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar_1309 from Narosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the PHA synthase comprises or consists of one or more of polypeptides having at least about 70%, at least about 75%, at least about 80%, at least about 85%, at least about 90%, at least about 95%, at least about 98%, or at least about 99% sequence identity to at least one of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the engineered microorganism further comprises or consists of an engineered pathway for producing a hydroxyisobutyric acid (HIBA) from the feedstock. In some embodiments, the feedstock comprises or consists of methane, ethane, propane, methanol, ethanol, propanol, and combinations thereof. In some embodiments, the HIBA comprises or consists of 2-hydroxyisobutyric acid (2-HIBA) and/or 3-hydroxyisobutyric acid (3-HIBA).

In some embodiments, the engineered pathway comprises or consists of one or more MMO, ADH, ACDH, and/or acetyl-CoA synthase (acs). In some embodiments, the engineered pathway further comprises or consists of one or more sleeping beauty mutase (Sbm). In some embodiments, the engineered pathway further comprises or consists of one or more methylmalonyl-CoA reductase (mmcr). In some embodiments, the engineered pathway comprises or consists of modifying one or more endogenous enzymes.

In some embodiments, the engineered microorganism is Escherichia coli.

In some embodiments, the method further comprises or consists of (i) separating the microorganism from the nutrient medium and (ii) optionally extracting the poly(HIBA) from the microorganism; and (iii) heating the poly(HIBA) to a temperature in a range from about 150° C. to about 450° C. for a time period from about 0.5 to 120 minutes to produce methacrylic acid (MAA). In some embodiments, the method further comprises or consists of esterifying the MAA with an alcohol to produce a methacrylate ester (MAE). In some embodiments, the method further comprises or consist of separating the poly(HIBA) from the nutrient medium; depolymerizing the poly(HIBA) to HIBA; and converting the HIBA using a catalyst to produce a methacrylic acid (MAA).

### Culturing

Microorganisms can be cultured using liquid or solid media as a source of nutrients for the microorganisms. Some microorganisms can be cultured in defined media, in which the liquid or solid media are generated by preparation using purified chemical components. The composition of the culture media can be adjusted to suit the microorganism or the industrial purpose for the culture.

Culturing parameters may include, but not be limited to, such features as the temperature of the culture media, the dissolved oxygen concentration, the dissolved carbon dioxide concentration, the rate of stirring of the liquid media, the pressure in the vessel, etc.

In some embodiments, the method further comprises of consists of separating the microorganism from the medium. In some embodiments, the method further comprises or consists of heating the poly(HIBA) to a temperature in a range of from about 150° C. to about 450° C. In some embodiments, heating is performed between about 0.5 to about 120 minutes.

The thermal decomposition of poly(HIBA) into MAA can be achieved by heating the polymer to sufficiently high temperatures. A method to convert poly(3-hydroxypropionate) into acrylic acid was described by Metabolix et al. (See, International Patent WO2013185009A1, which is incorporated by reference in its entirety herein, including any drawings). A similar process would be applicable to conversion of poly(HIBA) into methacrylic acid (MAA), as described in this patent application, Example 9.

In some embodiments, MAA is produced. In some embodiments, MAA is esterified with an alcohol to produce MAE. In some embodiments, poly(HIBA) is separated from the nutrient medium. In some embodiments, poly(HIBA) is depolymerized to HIBA. In some embodiments, HIBA is converted to MAA with a catalyst.

Performing the thermal decomposition/pyrolysis in the presence of methanol, ethanol, or other alcohol can result in the formation of an ester of the methacrylic acid molecule with the alcohol, such as methyl methacrylate, ethyl methacrylate, etc. The entire contents of the International Patent WO2013185009A1 are incorporated herein by reference for all purposes and relied upon.

### Nucleic Acid(s)

A third aspect provides a nucleic acid comprising or consisting of one or more first polynucleotides encoding a CoA-ligase and/or a polyhydroxyalkanoate (PHA) polymerase in a first engineered pathway that catalyze a conversion of a feedstock to a poly(HIBA). Nucleic acids are composed of nucleotides, which are the monomers made of three components: a 5-carbon sugar, a phosphate group, and a nitrogenous base. The two main classes of nucleic acids are deoxyribonucleic acid (DNA) and ribonucleic acid (RNA). If the sugar is ribose, the polymer is RNA; if the sugar is the ribose derivative deoxyribose, the polymer is DNA. Nucleic acids are naturally occurring chemical compounds that serve as the primary information-carrying molecules in cells and make up the genetic material.

Nucleic acids are found in abundance in all living things, where they create, encode, and then store information of every living cell of every life-form on earth. In turn, they function to transmit and express that information inside and outside the cell nucleus to the interior operations of the cell and ultimately to the next generation of each living organism. Encoded information is contained and conveyed via the nucleic acid sequence, which provides the ‘ladder-step’ ordering of nucleotides within the molecules of RNA and DNA. Nucleic acids play an especially important role in directing protein synthesis.

A peptide is a short chain of amino acids linked by peptide bonds. Chains of fewer than ten or fifteen amino acids are called oligopeptides and include dipeptides, tripeptides, and tetrapeptides. Peptides fall under the broad chemical classes of biological polymers and oligomers, alongside nucleic acids, oligosaccharides, polysaccharides, and others.

A polypeptide is normally a longer, continuous, unbranched peptide chain. A polypeptide that contains more than approximately fifty amino acids is known as a “protein.” “Proteins” consist of one or more polypeptides arranged in a biologically functional way, often bound to ligands such as coenzymes and cofactors, or to another protein or other macromolecule such as DNA or RNA, or to complex macromolecular assemblies.

The nucleic acids may encode any of the proteins set forth herein. For example, the nucleic acids may encode one or more CoA-ligase. In some embodiments, the one or more CoA-ligase comprises or consists of one or more of isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar_1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12).

The nucleic acids may further encode or more PHA synthase. In some embodiments, the one or more PHA synthase comprises or consists of one or more of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

In some embodiments, the nucleic acid construct further comprises or consists of one or more second elements encoding enzymes in an engineered HIBA pathway (or engineered HIBA pathway enzymes) that catalyze a conversion of the feedstock to a hydroxyisobutyric acid (HIBA). In some embodiments, the feedstock comprises or consists of methane, ethane, propane, methanol, ethanol, propanol, and combinations thereof. In some embodiments, the HIBA comprises or consists of 2-hydroxyisobutyric acid (2-HIBA) and/or 3-hydroxyisobutyric acid (3-HIBA).

In some embodiments, the one or more second elements are part of or the same as the one or more first polynucleotides. In some embodiments, the one or more second elements are one or more second polynucleotides different from the one or more first polynucleotides.

In some embodiments, the engineered HIBA pathway comprises or consists of MMO, ADH, ACDH, and/or acetyl-CoA synthase. In some embodiments, the engineered HIBA pathway further comprises or consists of a sleeping beauty mutase (Sbm). In some embodiments, the engineered HIBA pathway further comprises or consists of a methylmalonyl-CoA reductase (mmcr). In some embodiments, the engineered pathway comprises or consists of modifying one or more endogenous enzymes.

In some embodiments, the one or nucleic acids express a protein in an engineered microorganism in a sufficient amount. In some embodiments, the engineered microorganism is Escherichia coli.

In some embodiments, the one or more CoA-ligase comprises or consists of one or more of polypeptides having at least about 70%, at least about 75%, at least about 80%, at least about 85%, at least about 90%, at least about 95%, at least about 98%, or at least about 99% sequence identity to at least one of Isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar_1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12). In some embodiments, the one or more PHA synthase comprises or consists of one or more of polypeptides having at least about 70%, at least about 75%, at least about 80%, at least about 85%, at least about 90%, at least about 95%, at least about 98%, or at least about 99% sequence identity to at least one of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

The one or more polynucleotide may be inserted or integrated into the genome of a microorganism. In some embodiments, the one or more nucleotides are modified. It will be recognized by one skilled in the art that absolute identity to the one or more polypeptides or one or more nucleotides is not strictly necessary. For example, changes in a particular gene or polynucleotide comprising a sequence encoding a polypeptide or the one or more polypeptides can be performed and screened for activity as set forth above. Such modified or mutated polynucleotides and polypeptides can be screened for expression or function using methods known in the art and as set forth above in the methods related to directed evolution. Such modified or mutated polynucleotides and polypeptides are intended to be within the scope of the current disclosure.

Those of skill in the art will recognize that, due to the degenerate nature of the genetic code, a variety of polynucleotides differing in their nucleotide sequences can be used to encode one or more genes native to the microorganism, culture, or engineered microorganism or one or more polypeptides of the disclosure. Due to the inherent degeneracy of the genetic code, other polynucleotides, which encode substantially the same or functionally equivalent polypeptides, can also be used. The disclosure includes polynucleotides of any sequence that encode the amino acid sequences of the polypeptides and proteins of the one or more polypeptides utilized in the methods of the disclosure.

In similar fashion, a polypeptide can typically tolerate one or more amino acid substitutions, deletions, and insertions in its amino acid sequence without loss or significant loss of a desired activity. The disclosure includes such one or more polypeptides with different amino acid sequences from the specific proteins described herein so long as the modified or variant polypeptides have an activity that is identical or similar to the referenced polypeptide. Accordingly, the amino acid sequences encoded by the polynucleotide sequences shown herein merely illustrate embodiments of the disclosure.

The disclosure also includes one or more polypeptides with different amino acid sequences than the specific proteins described herein if the modified or variant polypeptides have an activity that is desirable yet different from referenced polypeptide. In some embodiments, an enzyme may be altered by modifying the gene that encodes the enzyme so that the expressed protein is more or less active than the wild type version.

As will be understood by those of skill in the art, it can be advantageous to modify a coding sequence to enhance expression in a particular host, such as, without limitation, Escherichia coli. The genetic code is redundant with 64 possible codons, but most organisms typically use a subset of these codons. Codons can be substituted, without any resultant change to the amino acid sequence of the corresponding protein, to increase or decrease the translation rate of the sequence, in a process sometimes called “codon optimization”. The tables set forth herein are annotated to show some of the codon optimized sequences disclosed herein.

Optimized coding sequences can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. In addition, homologs of polynucleotides or enzymes or the proteins encoded by the one or more polynucleotides are encompassed by the disclosure.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

It is recognized that residue positions that are not identical often differ by conservative amino acid substitutions. In cases where two or more amino acid sequences differ from each other by conservative substitutions, the percent sequence identity or degree of homology may practically be adjusted upwards to correct for the conservative nature of the substitution. Means for making this adjustment are well known to those of skill in the art (See, e.g., Pearson W. R., 1994, Methods in Mol Biol 25: 365-89, which is incorporated in its entirety herein).

Sequence homology and sequence identity for polypeptides is typically measured using sequence analysis software. A typical algorithm used to compare a molecular sequence to a database containing a large number of sequences from different organisms is the computer program BLAST. When searching a database containing sequences from a large number of different organisms, it is typical to compare amino acid sequences.

Furthermore, any of the one or more polynucleotides native to the microbe or microorganism, culture, or engineered microorganism or genes encoding the enzymes or one or more polypeptides or genes native to the native microorganism, culture, or engineered microorganism (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast, bacteria, or any other suitable cell or organism.

For example, amino acid sequence variants of the one or more polypeptides can be prepared by mutations in the DNA. Methods for mutagenesis and nucleotide sequence alterations include, for example, Kunkel, (1985) Proc Natl Acad Sci USA 82:488-92; Kunkel, et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff, et al., (1978) Atlas of Protein Sequence and Structure (Natl Biomed Res Found, Washington, D.C.), each of which is incorporated by reference in their entirety.

Techniques known to those skilled in the art may be suitable to identify additional homologous (or otherwise analogous) genes and homologous (or otherwise analogous) enzymes. Generally, analogous genes and/or analogous enzymes can be identified by functional analysis and will have functional similarities. As an example, to identify homologous or analogous biosynthetic pathway genes, proteins, or enzymes, techniques may include, but are not limited to, cloning a gene by PCR using primers based on a published sequence of a gene/enzyme of interest or by degenerate PCR using degenerate primers designed to amplify a conserved region among a gene of interest.

Further, one skilled in the art can use techniques to identify homologous or analogous genes, proteins, or enzymes with functional homology or similarity. Techniques include examining a cell or cell culture for the catalytic activity of an enzyme through in vitro enzyme assays for the activity (e.g. as described herein or in Kiritani, K., Branched-Chain Amino Acids Methods Enzymology, 1970), then isolating the enzyme with the activity through purification, determining the protein sequence of the enzyme through techniques such as Edman degradation, design of PCR primers to the likely nucleic acid sequence, amplification of the DNA sequence through PCR, and cloning of the nucleic acid sequence. To identify homologous or similar genes and/or homologous or similar proteins, analogous genes and/or analogous proteins, techniques also include comparison of data concerning a candidate gene or enzyme with databases such as BRENDA, KEGG, or MetaCYC. The candidate gene or proteins may be identified within the above-mentioned databases in accordance with the teachings herein.

In some embodiments, the microorganism, culture, or engineered microorganism expressing one or more polypeptides has one or more genes native to the microorganism, culture, or engineered microorganism that have been genetically modified, deleted, or whose expression has been reduced or eliminated. Reduction or elimination of expression may occur through any method known to one skilled in the art and all ways of genetically modifying, deleting, and/or of reducing or eliminating expression of genes native to the microorganism, culture, or engineered microorganism are provided herein. In particular, one skilled in the art will understand that any form of genetic alteration or genetic engineering or genetic modification, may be used as an alternative to deletion. In some embodiments, other forms of genetic modification that may be used as an alternative to deletion include, for example, without limitation, gene knockouts, mutation, gene targeting, homologous recombination, gene knockdown, gene silencing, gene addition, molecular cloning, gene attenuation, genome editing, CRISPR interference, or any technique that may be used to suppress or alter or enhance a particular phenotype.

In some embodiments, the polynucleotide native to the microbe, culture, or engineered microorganism can be altered in other ways, including, but not limited to, expressing a modified form of a polypeptide where the modified form of the polypeptide exhibits increased or decreased solubility in the microorganism or engineered microorganism, expressing an altered form of a polypeptide that lacks a domain through which activity is inhibited, or expressing an altered form of a polypeptide that is more or less affected by feed-back or feed-forward regulation by another molecule in a pathway expressed in the microorganism, culture, or engineered microorganism. In some embodiments, the strength of the promoter, enhancer, or operator to which the nucleotide sequence for the one or more genes native to the microorganism, culture, or engineered microorganism is operably linked may also be manipulated, decreased, or increased or different promoters, enhancers, or operators may be introduced.

### Expression of Nucleic Acid(s)

Expression of one or more polynucleotides in one or more engineered microorganisms can be accomplished by introducing one or more exogenous polynucleotides into a microorganism or culturing a nucleic acid comprising a nucleotide sequence encoding one or more polypeptides under the control of regulatory elements that permit expression in the microorganism or culture.

Nucleic acids can be introduced into a microorganism or culture by any method known to one of skill in the art without limitation (See, for example, Hinnen et al. (1978) Proc. Natl. Acad. Sci. USA 75:1292-3; Cregg et al. (1985) Mol. Cell. Biol. 5:3376-3385; Goeddel et al. eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY, each of which is incorporated by reference in its entirety herein). Exemplary techniques include, but are not limited to, spheroplasting, electroporation, PEG 1000 mediated transformation, and lithium acetate- or lithium chloride-mediated transformation.

In some embodiments, the nucleic acid comprises or consists of one or more plasmids. In some embodiments, the nucleic acid comprises or consists of one or more extrachromosomal plasmids. In some embodiments, the nucleic acid is a chromosomal integration vector that can integrate the nucleotide sequence into the chromosome of the microorganism or culture.

### Modification

Engineered microorganisms provided herein comprise or consist of a CoA-ligase and a PHA polymerase, capable of producing a poly(HIBA) from a feedstock. One skilled in the art would be able to produce the engineered microorganisms according to the methods set forth herein. Expression of genes and genomes may be modified. In some embodiments, expression of the one of more polynucleotides is modified. For example, the copy number of an enzyme or one of more polynucleotides in a microorganism or culture may be altered by modifying the transcription of the gene that encodes a polypeptide. This can be achieved, for example, by modifying the copy number of the nucleotide sequence encoding the one of more polynucleotides (e.g., by using a higher or lower copy number expression vector comprising the nucleotide sequence, or by introducing additional copies of the nucleotide sequence into the genome of the microorganism or culture, or by introducing additional nucleotide sequences into the genome of the microorganism or culture that express the same or similar polypeptide, or by genetically modifying or deleting or disrupting the nucleotide sequence in the genome of the microorganism or culture), by changing the order of coding sequences on a polycistronic mRNA of an operon, or by breaking up an operon into individual genes, each with its own control elements. The strength of the promoter, enhancer, or operator to which the nucleotide sequence is operably linked may also be manipulated or increased or decreased or different promoters, enhancers, or operators may be introduced.

Alternatively, or in addition, the copy number of one or more polypeptides may be altered by modifying the level of translation of an mRNA that encodes one or more polypeptides. This can be achieved, for example, by modifying the stability of the mRNA, modifying the sequence of the ribosome binding site, modifying the distance or sequence between the ribosome binding site and the start codon of the enzyme coding sequence, modifying the entire intercistronic region located upstream of or adjacent to the 5′ side of the start codon of the enzyme coding region, stabilizing the 3′-end of the mRNA transcript using hairpins and specialized sequences, modifying the codon usage of an enzyme, altering expression of rare codon tRNAs used in the biosynthesis of the enzyme, and/or increasing the stability of an enzyme, as, for example, via mutation of its coding sequence.

Expression of one or more polynucleotides may be modified or regulated by targeting particular genes. For example, without limitation, in some embodiments of the methods described herein, a microorganism, culture, or engineered microorganism is contacted with one or more nucleases capable of cleaving, i.e., causing a break at a designated region within a selected site. In some embodiments, the break is a single-stranded break, that is, one but not both strands of the target site is cleaved. In some embodiments, the break is a double-stranded break. In some embodiments, a break-inducing agent is used. A break-inducing agent is any agent that recognizes and/or binds to a specific polynucleotide recognition sequence to produce a break at or near a recognition sequence. Examples of break-inducing agents include, but are not limited to, endonucleases, site-specific recombinases, transposases, topoisomerases, and zinc finger nucleases, and include modified derivatives, variants, and fragments thereof.

In some embodiments, a recognition sequence within a selected target site can be endogenous or exogenous to a microorganism, culture, or engineered microorganism's genome. When the recognition site is an endogenous or exogenous sequence, it may be a recognition sequence recognized by a naturally occurring, or native break-inducing agent. Alternatively, an endogenous or exogenous recognition site could be recognized and/or bound by a modified or engineered break-inducing agent designed or selected to specifically recognize the endogenous or exogenous recognition sequence to produce a break. In some embodiments, the modified break-inducing agent is derived from a native, naturally occurring break-inducing agent. In other embodiments, the modified break-inducing agent is artificially created or synthesized. Methods for selecting such modified or engineered break-inducing agents are known in the art.

In some embodiments, the one or more nucleases is a CRISPR/Cas-derived RNA-guided endonuclease. CRISPR may be used to recognize, genetically modify, and/or silence genetic elements at the RNA or DNA level or to express heterologous or homologous genes. CRISPR may also be used to regulate endogenous or exogenous nucleic acids. Any CRISPR/Cas system known in the art finds use as a nuclease in the methods and compositions provided herein. CRISPR systems that find use in the methods and compositions provided herein also include those described in International Publication Numbers WO 2013/142578 A1, WO 2013/098244 A1 and Nucleic Acids Res (2017) 45 (1): 496-508, the contents of which are hereby incorporated in their entireties.

In some embodiments, the one or more nucleases is a TAL-effector DNA binding domain-nuclease fusion protein (TALEN). TAL effectors of plant pathogenic bacteria in the genus Xanthomonas play important roles in disease, or trigger defense, by binding host DNA and activating effector-specific host genes. (See, e.g., Gu et al. (2005) Nature 435:1122-5; Yang et al., (2006) Proc. Natl. Acad. Sci. USA 103:10503-8; Kay et al., (2007) Science 318:648-51; Sugio et al., (2007) Proc. Natl. Acad. Sci. USA 104:10720-5; Romer et al., (2007) Science 318:645-8; Boch et al., (2009) Science 326(5959):1509-12; and Moscou and Bogdanove, (2009) 326(5959):1501, each of which is incorporated by reference in their entirety). A TAL effector comprises a DNA binding domain that interacts with DNA in a sequence-specific manner through one or more tandem repeat domains. The repeated sequence typically comprises 34 amino acids, and the repeats are typically 91-100% homologous with each other. Polymorphism of the repeats is usually located at positions 12 and 13, and there appears to be a one-to-one correspondence between the identity of repeat variable-diresidues at positions 12 and 13 with the identity of the contiguous nucleotides in the TAL-effector's target sequence.

The TAL-effector DNA binding domain may be engineered to bind to a desired sequence and fused to a nuclease domain, e.g., from a type II restriction endonuclease, typically a nonspecific cleavage domain from a type II restriction endonuclease such as FokI (See, e.g., Kim et al. (1996) Proc. Natl. Acad. Sci. USA 93:1156-1160, which is incorporated by reference in its entirety herein). Other useful endonucleases may include, for example, HhaI, HindIII, Nod, BbvCI, EcoRI, BglI, and AlwI. Thus, in some embodiments, the TALEN comprises a TAL effector domain comprising a plurality of TAL effector repeat sequences that, in combination, bind to a specific nucleotide sequence in a target DNA sequence, such that the TALEN cleaves target DNA within or adjacent to the specific nucleotide sequence. TALENS useful for the methods provided herein include those described in WO10/079430 and U.S. Patent Application Publication No. 2011/0145940, which is incorporated by reference herein in its entirety.

In some embodiments, the one or more of the nucleases is a zinc-finger nuclease (ZFN). ZFNs are engineered break-inducing agents comprised of a zinc finger DNA binding domain and a break-inducing agent domain. Engineered ZFNs consist of two zinc finger arrays (ZFAs), each of which is fused to a single subunit of a non-specific endonuclease, such as the nuclease domain from the FokI enzyme, which becomes active upon dimerization.

Useful zinc-finger nucleases include those that are known and those that are engineered to have specificity for one or more sites. Zinc finger domains are amenable for designing polypeptides which specifically bind a selected polynucleotide recognition sequence. Thus, they are amenable to modifying or regulating expression by targeting particular genes.

### Chaperones

Some embodiments further comprise one or more chaperones. Protein folding chaperones are proteins that improve the folding of polypeptide (amino acid) chains into 3-dimensional structures. Protein folding chaperones help their substrates, namely other proteins, to become properly folded and often more highly soluble. Since most proteins must be folded in a particular shape to be functional, the expression of protein folding chaperones can assist in the proper assembly of certain enzymes in a cell and thereby can result in an increase in the enzymatic activity of the substrate proteins.

In some embodiments, the at least one polynucleotide comprises or consists of one or more modifications. In some embodiments, the one or more modifications comprises or consists of polynucleotides encoding, and capable of expressing, one or more chaperone protein. In some embodiments, the one or more chaperone protein comprises or consists of groEL and/or groES. In some embodiments, the groEL and/or groES are Escherichia coli groEL and/or groES, Methylococcus capsulatus groEL and/or groES, or both. In some embodiments, the one or more chaperones comprise one or more polypeptides, each of the one or more polypeptides having an amino acid sequence, the amino acid sequence being more than about 70%, more than about 75%, more than about 80%, more than about 85%, more than about 90%, more than about 95%, or more than about 99% identical or identical to any of one of SEQ ID NOs: 62 to 66, respectively.

### EXAMPLES

### Example 1

### In Vivo Polymerization of 3-HIBA

This study evaluates the activity of a panel of strains against substrates 3-HIBA for in vivo polymerization of 3-HIBA and further conversion of poly(HIBA) to methacrylic acid (MAA).

In this study, a panel of strains was constructed containing two plasmids, one expressing a CoA-ligase and one expressing a polyhydroxyalkanoate (PHA) synthase.

Strain NH283 is a strain of E. coli bacteria (NEB Express ΔcaraBAD::cat) and was constructed as described in publication WO2017087731A1, paragraph [0153].

Strain LC706 is equivalent to strain BW25113 (CGSC 7636, “Datsenko, KA, BL Wanner 2000. One-step inactivation of chromosomal genes in Escherichia coli K-12 using PCR products,”Proc. Natl. Acad. Sci. U.S.A. 97(12):6640-5.”), which is a standard, widely-available strain of E. coli K-12.

The strains tested in this study are shown in Table 5 below. These strains were cultured under conditions where 3-HIBA was present and then assayed for poly(3-HIBA) and methacrylic acid (MAA).

The panel of strains was inoculated into deep-well 96-well culture plates with 500 μL of LB supplemented with carbenicillin (100 μg/mL) and kanamycin (50 μg/mL). The plate was covered with a breathable seal (Nunc Breathe-Easier) and incubated at 37° C. and 800 rpm for 16 hours. All the strains on this plate were subcultured into 500 μt of LB supplemented with carbenicillin (100 μg/mL) and kanamycin (50 μg/mL) starting with approximately 5 μL of the overnight culture. These strains were cultured at 37° C. and 800 rpm for 24 hours. These strains were subcultured by pipetting 5 μL of each overnight culture into 500 μt of a media composed of LB supplemented with a 1:10 dilution of 20×PBS (final concentration 2×PBS), 0.25% (w/v) racemic 3-hydroxyisobutyrate (sodium salt), 10 g/L glycerol, carbenicillin (100 μg/mL) and kanamycin (50 μg/mL). This plate was covered with a breathable seal and placed in an air-tight container and incubated at 37° C. and 900 rpm for 4 days. These strains tested in this study are shown in Table 5 below.

### PHA Base Digest Protocol

0.5 to 1.5 mL of cell culture samples were collected in either 1.5 mL polypropylene tubes or 2 mL polypropylene 96-well plates and spun down in a table-top centrifuge to collect solid biomass. The supernatant was discarded. The resulting cell pellets were re-suspended in 1 mL of cold water and spun down again. This wash step was repeated a minimum of three times. After the final wash, the solid biomass pellets were covered with Nunc breath-easier nylon breathable seals and placed in an oven at 55° C. to dry for 48 to 72 hours. 200 μL of aqueous 1 N sodium hydroxide solution was added to each dried pellet, covered either with a polypropylene tube lid or silicone plate mat, and incubated in a 70-80° C. oven for 4 hours. Samples were then removed, cooled, and neutralized with 200 μL of aqueous 1 N hydrochloric acid. 150 μL of each sample was then transferred to a 96-well PTFE plate filter and mixed with 15 μL of 100 mM citric acid. Samples were spun through the filter and collected in Corning Costar polypropylene 96-well plates, sealed with zone-free polyethylene plate seals (Excel Scientific), and placed in an Agilent 1290 HPLC system for analysis.

For HPLC analysis, 20 μL of each sample was separated using a Biorad Aminex HPX-87H column and 0.008 N sulfuric acid as a mobile phase at a flow-rate of 0.6 mL/min. Effluent was detected via both DAD and RID and compared to a set of prepared standards of 3-hydroxyisobutyrate (racemic) and methacrylic acid. From this comparison, rough estimates of poly-3-hydroxyisobutyrate were calculated assuming any of these species measured from dry biomass corresponded to digested polymer. Agilent software was used to integrate the peaks corresponding to the digested polymer in order to provide a quantitative measurement of the detected molecular species.

A background level was determined by analyzing samples containing only the media or strains that did not contain both a CoA-ligase and a PHA synthase enzyme.

The test results are shown in Table 5 below. As shown in Table 5, a number of strains exhibited significant peaks at both the 3-HIBA and MAA retention times, indicating the presence of poly(3-HIBA) in the cells. These peaks were not present or above a background level in the control strains. The following strains showed significant signals in this assay: sTRiM0256, sTRiM0404, sTRiM0470 (redundant with sTRiM0256), sTRiM0249, sTRiM0250, sTRiM0459, sTRiM0180, sTRiM0257, sTRiM0426, sTRiM0179, sTRiM0222, sTRiM0258, sTRiM0214, sTRiM0447, sTRiM0397, and sTRiM0251, as shown in Table 5 below. Applicant surprisingly found that certain combinations of enzymes showed significant signals in this assay, while some other combinations demonstrated no activity in producing poly(3-HIBA) or MAA, as shown in Table 5.

TABLE 5
The test results of the 3-HIBA and MAA screen area to indicate the activities
of a panel of strains (each of which was constructed containing two plasmids,
one expressing a CoA-ligase and one expressing a polyhydroxyalkanoate (PHA)
synthase) against substrates 3-HIBA to catalyze the polymerization of 3-HIBA
to poly(3-HIBA) and further conversion poly(3-HIBA) to methacrylic acid (MAA).

| | | | | 3HIBA |
|---|---|---|---|---|
| | | | | Screen Area |
| | | | | (Methacrylic |
| Strain | Strain Details | Ligase Gene | Polymerase Gene | Acid - A.U.) |
| sTRiM0256 | NH283 pNH306 | hadA (C. difficile) | phaC-phaE (A. | 54486 |
| | pNH299 | | vinosum) | |
| sTRiM0404 | NH283 pNH321 | pcICS (P. | phaC-phaE (A. | 44483 |
| | pNH306 | chlororaphis) | vinosum) | |
| sTRiM0470 | NH283 pNH299 | hadA (C. difficile) | phaC-phaE (A. | 25318 |
| | pNH306 | | vinosum) | |
| sTRiM0249 | LC706 pNH306 | hadA (C. difficile) | phaC-phaE (A. | 22838 |
| | pNH299 | | vinosum) | |
| sTRiM0250 | LC706 pNH306 | hadA (C. difficile, | phaC-phaE (A. | 21117 |
| | pNH299b | codon optimized) | vinosum) | |
| sTRiM0459 | NH283 pNH326 | Nmar_1309 (N. | phaC-phaE (A. | 17488 |
| | pNH306 | maritimus SCM1) | vinosum) | |
| sTRiM0180 | LC706 pNH305 | hadA (C. difficile, | phaC1437 | 17273 |
| | pNH299b | codon optimized) | (Pseudomonas sp.) | |
| sTRiM0257 | NH283 pNH306 | hadA (C. difficile, | phaC-phaE (A. | 11854 |
| | pNH299b | codon optimized) | vinosum) | |
| sTRiM0426 | NH283 pNH323 | 3HP-CoA synthetase | phaC-phaE (A. | 11032 |
| | pNH306 | (M. sedula) | vinosum) | |
| sTRiM0179 | LC706 pNH305 | hadA (C. difficile) | phaC1437 | 10241 |
| | pNH299 | | (Pseudomonas sp.) | |
| sTRiM0222 | NH283 pNH305 | hadA (C. difficile, | phaC1437 | 5443 |
| | pNH299b | codon optimized) | (Pseudomonas sp.) | |
| sTRiM0258 | NH283 pNH306 | HCL (A. | phaC-phaE (A. | 3865 |
| | pNH300 | tertiaricarbonus | vinosum) | |
| | | L108) | | |
| sTRiM0214 | NH283 pNH304 | hadA (C. difficile) | phaC1 | 1819 |
| | pNH299 | | (Chromobacterium | |
| | | | USM2) | |
| sTRiM0447 | NH283 pNH325 | acs (putative, S. | PHA polymerase 3 | 1696 |
| | pNH336 | sulfaraticus) | (R. opacus PD630) | |
| sTRiM0397 | NH283 pNH321 | pcICS (P. | phaC | 1556 |
| | pNH330 | chlororaphis) | (Betaproteobacteria | |
| | | | bacterium) | |
| sTRiM0251 | LC706 pNH306 | HCL (A. | phaC-phaE (A. | 1440 |
| | pNH300 | tertiaricarbonus | vinosum) | |
| | | L108) | | |
| sTRiM0471 | NH283 pNH311 | matB T207S (R. | phaC (C. huifangae) | 1310 |
| | pNH327 | trifoli) | | |
| sTRiM0173 | LC706 pNH304 | hadA (C. difficile, | phaC1 | 1249 |
| | pNH299b | codon optimized) | (Chromobacterium | |
| | | | USM2) | |
| sTRiM0245 | NH283 pNH309 | alkK (P. oleovorans) | phaC (A. caviae) | 1155 |
| | pNH301 | | | |
| sTRiM0460 | NH283 pNH299 | hadA (C. difficile) | phaC (C. huifangae) | 1084 |
| | pNH327 | | | |
| sTRiM0395 | NH283 pNH321 | pcICS (P. | phaC | 973 |
| | pNH328 | chlororaphis) | (Burkholderiales | |
| | | | bacterium) | |
| sTRiM0403 | NH283 pNH321 | pcICS (P. | PHA polymerase 3 | 952 |
| | pNH336 | chlororaphis) | (R. opacus PD630) | |
| sTRiM0172 | LC706 pNH304 | hadA (C. difficile) | phaC1 | 944 |
| | pNH299 | | (Chromobacterium | |
| | | | USM2) | |
| sTRiM0427 | NH283 pNH324 | 3HP-CoA synthetase | phaC (C. huifangae) | 915 |
| | pNH327 | (S. tokodaii) | | |
| sTRiM0415 | NH283 pNH322 | matB (S. coelicolor) | phaC-phaE (A. | 902 |
| | pNH306 | | vinosum) | |
| sTRiM0325 | LC706 | hadA (C. difficile, | phaE-phaC (Chang | 890 |
| | pNH299b | codon optimized) | lab metagenomics | |
| | pNH310 | | “CAP”) | |
| sTRiM0416 | NH283 pNH323 | 3HP-CoA synthetase | phaC (C. huifangae) | 820 |
| | pNH327 | (M. sedula) | | |
| sTRiM0414 | NH283 pNH322 | matB (S. coelicolor) | PHA polymerase 3 | 812 |
| | pNH336 | | (R. opacus PD630) | |
| sTRiM0446 | NH283 pNH325 | acs (putative, S. | PHA polymerase 2 | 809 |
| | pNH335 | sulfaraticus) | (R. opacus PD630) | |
| sTRiM0463 | NH283 pNH299 | hadA (C. difficile) | phaC | 794 |
| | pNH330 | | (Betaproteobacteria | |
| | | | bacterium) | |
| sTRiM0181 | LC706 pNH305 | HCL (A. | phaC1437 | 710 |
| | pNH300 | tertiaricarbonus | (Pseudomonas sp.) | |
| | | L108) | | |
| sTRiM0253 | LC706 pNH306 | orfZ (C. kluyveri) | phaC-phaE (A. | 702 |
| | pNH302 | | vinosum) | |
| sTRiM0223 | NH283 pNH305 | HCL (A. | phaC1437 | 677 |
| | pNH300 | tertiaricarbonus | (Pseudomonas sp.) | |
| | | L108) | | |
| sTRiM0244 | NH283 pNH309 | HCL (A. | phaC (A. caviae) | 615 |
| | pNH300 | tertiaricarbonus | | |
| | | L108) | | |
| sTRiM0472 | NH283 pNH311 | matB T207S (R. | phaC | 613 |
| | pNH328 | trifoli) | (Burkholderiales | |
| | | | bacterium) | |
| sTRiM0437 | NH283 pNH324 | 3HP-CoA synthetase | phaC-phaE (A. | 596 |
| | pNH306 | (S. tokodaii) | vinosum) | |
| sTRiM0215 | NH283 pNH304 | hadA (C. difficile, | phaC1 | 553 |
| | pNH299b | codon optimized) | (Chromobacterium | |
| | | | USM2) | |
| sTRiM0448 | NH283 pNH325 | acs (putative, S. | phaC-phaE (A. | 535 |
| | pNH306 | sulfaraticus) | vinosum) | |
| sTRiM0458 | NH283 pNH326 | Nmar_1309 (N. | PHA polymerase 3 | 491 |
| | pNH336 | maritimus SCM1) | (R. opacus PD630) | |
| sTRiM0402 | NH283 pNH321 | pcICS (P. | PHA polymerase 2 | 439 |
| | pNH335 | chlororaphis) | (R. opacus PD630) | |
| sTRiM0260 | NH283 pNH306 | orfZ (C. kluyveri) | phaC-phaE (A. | 413 |
| | pNH302 | | vinosum) | |
| sTRiM0324 | LC706 pNH299 | hadA (C. difficile) | phaE-phaC (Chang | 398 |
| | pNH310 | | lab metagenomics | |
| | | | “CAP”) | |
| sTRiM0376 | NH283 pNH319 | AtACS T324G (A. | phaC (Blastococcus | 374 |
| | pNH331 | thaliana) | sp. CT_GayMR19) | |
| sTRiM0461 | NH283 pNH299 | hadA (C. difficile) | phaC | 367 |
| | pNH328 | | (Burkholderiales | |
| | | | bacterium) | |
| sTRiM0326 | LC706 pNH300 | HCL (A. | phaE-phaC (Chang | 341 |
| | pNH310 | tertiaricarbonus | lab metagenomics | |
| | | L108) | “CAP”) | |
| sTRiM0438 | NH283 pNH325 | acs (putative, S. | phaC (C. huifangae) | 332 |
| | pNH327 | sulfaraticus) | | |
| sTRiM0450 | NH283 pNH326 | Nmar_1309 (N. | phaC | 325 |
| | pNH328 | maritimus SCM1) | (Burkholderiales | |
| | | | bacterium) | |
| sTRiM0449 | NH283 pNH326 | Nmar_1309 (N. | phaC (C. huifangae) | 282 |
| | pNH327 | maritimus SCM1) | | |
| sTRiM0192 | LC706 pNH308 | fadK (E. coli) | phaC-phaR (B. | 241 |
| | pNH298-1 | | megaterium) | |
| sTRiM0166 | LC706 pNH303 | hadA (C. difficile, | phaC1 (C. necator) | 238 |
| | pNH299b | codon optimized) | | |
| sTRiM0205 | NH283 pNH303 | prpE (E. coli) | phaC1 (C. necator) | 235 |
| | pNH297 | | | |
| sTRiM0425 | NH283 pNH323 | 3HP-CoA synthetase | PHA polymerase 3 | 235 |
| | pNH336 | (M. sedula) | (R. opacus PD630) | |
| sTRiM0309 | NH283 pNH311 | matB T207S (R. | phaC1437 | 221 |
| | pNH305 | trifoli) | (Pseudomonas sp.) | |
| sTRiM0191 | LC706 pNH308 | prpE (E. coli) | phaC-phaR (B. | 217 |
| | pNH297 | | megaterium) | |
| sTRiM0327 | LC706 pNH301 | alkK (P. oleovorans) | phaE-phaC (Chang | 209 |
| | pNH310 | | lab metagenomics | |
| | | | “CAP”) | |
| sTRiM0410 | NH283 pNH322 | matB (S. coelicolor) | phaC (B. cepacia) | 0 |
| | pNH332 | | | |
| sTRiM0430 | NH283 pNH324 | 3HP-CoA synthetase | phaC | 0 |
| | pNH330 | (S. tokodaii) | (Betaproteobacteria | |
| | | | bacterium) | |
| sTRiM0431 | NH283 pNH324 | 3HP-CoA synthetase | phaC (Blastococcus | 0 |
| | pNH331 | (S. tokodaii) | sp. CT_GayMR19) | |
| sTRiM0440 | NH283 pNH325 | acs (putative, S. | phaC (Rhizobacter | 0 |
| | pNH329 | sulfaraticus) | sp.) | |
| sTRiM0442 | NH283 pNH325 | acs (putative, S. | phaC (Blastococcus | 0 |
| | pNH331 | sulfaraticus) | sp. CT_GayMR19) | |
| sTRiM0476 | NH283 pNH311 | matB T207S (R. | phaC (B. cepacia) | 0 |
| | pNH332 | trifoli) | | |

### Example 2

### Conversion of 3-HIBA to Poly(3-HIBA) and Pyrolysis of Poly(3-HIBA) to MAA.

This study compares the activities of two specific strains sTRIM0256 and sTRIM0290 against substrates 3-HIBA for in vivo polymerization of 3-HIBA and further the conversion of poly(HIBA) to methacrylic acid (MAA).

Strain sTRIM0256 was constructed as described above in Example 1 and Table 5. This strain comprises an Escherichia coli bacterium with two plasmids constitutively expressing a CoA-ligase (hadA from Clostridium difficile, SEQ ID NO: 3) and a PHA synthase (phaC and phaE from Allochromatium vinosum, SEQ ID NO: 22 and 23). Strain sTRIM0290 is identical to sTRIM0256 but is lacking any PHA synthase. These strains were cultured under conditions where 3-HIBA was present and then assayed for poly(3-HIBA) and methacrylic acid (MAA).

Both strains were inoculated into 2 mL of LB supplemented with carbenicillin at a final concentration of 100 μg/mL and kanamycin at a final concentration of 50 μg/mL. These strains were incubated for 16 hours at 37° C., shaking at 280 rpm. From these cultures, 1 mL was transferred into 25 mL of LB supplemented with 2×PBS, 10 g/L glycerol, racemic 3-hydroxyisobutyric acid to a final concentration of 0.25% (w/v), carbenicillin (100 μg/mL), and kanamycin (50 μg/mL). These cultures were incubated in 125 mL baffled flasks at 37° C., shaking at 200 rpm for 4 days. After 4 days, the cultures were each split into 2 mL tubes and spun in a centrifuge at 13000 rpm. The supernatant was discarded. The samples were resuspended in 2 mL cold water, spun in the centrifuge, and the supernatant was discarded. The samples were washed similarly two more times in 1 mL of cold water. After the final wash, the tubes were left open to dry in an oven at 55° C. for 3 days.

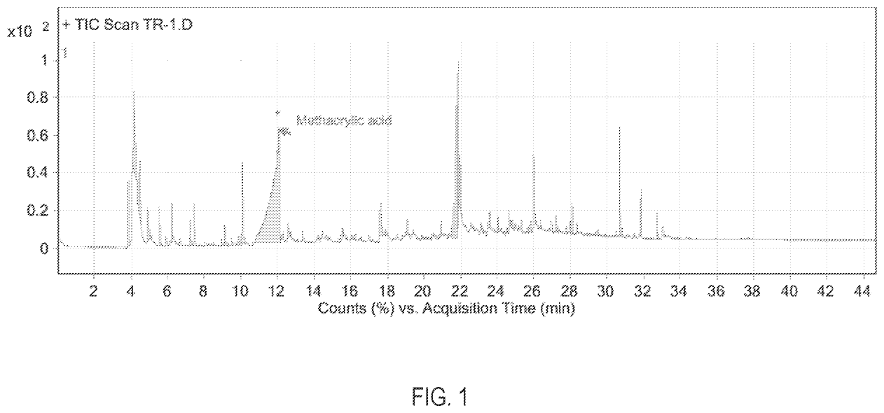

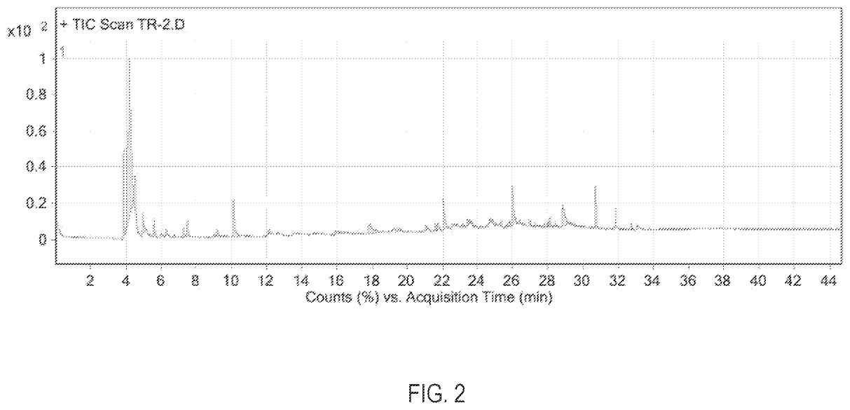

Samples were analyzed by pyrolysis gas chromatography-mass spectrometry (GCMS). The test results for strains sTRIM0256 and sTRIM0290 are shown in FIG. 1 and FIG. 2 respectively. The sample from strain sTRIM0256 resulted in a spectrum shown in FIG. 1 which exhibited a significant peak that corresponded to methacrylic acid (MAA).

The peak that appeared at the acquisition time between 10 minutes to 12 minutes was identified to be methacrylic acid via pyrolysis GCMS. The control strain sTRIM0290 did not result in a peak that corresponded to methacrylic acid (MAA) as shown in FIG. 2.

The test results in FIG. 1 and FIG. 2 clearly demonstrate that strain sTRIM0256 have high activity in catalyzing the conversion of 3-HIBA to poly(3-HIBA), while sTRIM0290 does not have the activity in catalyzing the polymerization of 3-HIBA.

This disclosure provides various discussions and information about many features relating to microorganisms capable of producing poly(HIBA) from feedstocks and method of producing methacrylic acid (MAA) and methacrylate esters (MAE) from feedstocks.

### Example S: Sequences

Table S provides sequences referred to herein in the present specification.

TABLE S
Sequences.

| SEQ ID | Molecule/ | | |
|---|---|---|---|
| NO | Gene Name | Organism | Sequence |
| 1 | prpE | E. coli | MSFSEFYQRSINEPEQFWAEQARRIDWQTPFTQTLDHSNPP |
| | | | FARWFCEGRTNLCHNAIDRWLEKQPEALALIAVSSETEEER |
| | | | TFTFRQLHDEVNAVASMLRSLGVQRGDRVLVYMPMIAEAHI |
| | | | TLLACARIGAIHSVVFGGFASHSVAARIDDAKPVLIVSADA |
| | | | GARGGKIIPYKKLLDDAISQAQHQPRHVLLVDRGLAKMARV |
| | | | SGRDVDFASLRHQHIGARVPVAWLESNETSCILYTSGTTGK |
| | | | PKGVQRDVGGYAVALATSMDTIFGGKAGGVFFCASDIGWVV |
| | | | GHSYIVYAPLLAGMATIVYEGLPTWPDCGVWWKIVEKYQVS |
| | | | RMFSAPTAIRVLKKFPTAEIRKHDLSSLEVLYLAGEPLDEP |
| | | | TASWVSNTLDVPVIDNYWQTESGWPIMAIARGLDDRPTRLG |
| | | | SPGVPMYGYNVOLLNEVTGEPCGVNEKGMLVMEGPLPPGCI |
| | | | QTIWGDDGRFVKTYWSLFSRPVYATFDWGIRDADGYHFILG |
| | | | RTDDVINVAGHRLGTREIEESISSHPGVAEVAVVGVKDALK |
| | | | GQVAVAFVIPKESDSLEDRDVAHSQEKAIMALVDSQIGNFG |
| | | | RPAHVWFVSQLPKTRSGKMLRRTIQAICEGRDPGDLTTIDD |
| | | | PASLDQIRQAMEE |
| 2 | fadK | E. coli | MHPTGPHLGPDVLFRESNMKVTLTFNEQRRAAYRQQGLWGD |
| | | | ASLADYWQQTARAMPDKIAVVDNHGASYTYSALDHAASCLA |
| | | | NWMLAKGIESGDRIAFQLPGWCEFTVIYLACLKIGAVSVPL |
| | | | LPSWREAELVWVLNKCQAKMFFAPTLFKQTRPVDLILPLQN |
| | | | QLPQLQQIVGVDKLAPATSSLSLSQIIADNTSLTTAITTHG |
| | | | DELAAVLFTSGTEGLPKGVMLTHNNILASERAYCARLNLTW |
| | | | QDVFMMPAPLGHATGFLHGVTAPFLIGARSVLLDIFTPDAC |
| | | | LALLEQQRCTCMLGATPFVYDLLNVLEKQPADLSALRFFLC |
| | | | GGTTIPKKVARECQQRGIKLLSVYGSTESSPHAVVNLDDPL |
| | | | SRFMHTDGYAAAGVEIKVVDDARKTLPPGCEGEEASRGPNV |
| | | | FMGYFDEPELTARALDEEGWYYSGDLCRMDEAGYIKITGRK |
| | | | KDIIVRGGENISSREVEDILLQHPKIHDACVVAMSDERLGE |
| | | | RSCAYVVLKAPHHSLSLEEVVAFFSRKRVAKYKYPEHIVVI |
| | | | EKLPRTTSGKIQKFLLRKDIMRRLTQDVCEEIE |
| 3 | hadA | Clostridium | MLLEGVKVVELSSFIAAPCCAKMLGDWGAEVIKIEPIEGDG |
| | | difficile | IRVMGGTFKSPASDDENPMFELENGNKKGVSINVKSKEGVE |
| | | | ILHKLLSEADIFVTNVRVQALEKMGIAYDQIKDKYPGLIFS |
| | | | QILGYGEKGPLKDKPGFDYTAYFARGGVSQSVMEKGTSPAN |
| | | | TAAGFGDHYAGLALAAGSLAALHKKAQTGKGERVTVSLFHT |
| | | | AIYGMGTMITTAQYGNEMPLSRENPNSPLMTTYKCKDGRWI |
| | | | QLALIQYNKWLGKFCKVINREYILEDDRYNNIDSMVNHVED |
| | | | LVKIVGEAMLEKTLDEWSALLEEADLPFEKIQSCEDLLDDE |
| | | | QAWANDFLFKKTYDSGNTGVLVNTPVMFRNEGIKEYTPAPK |
| | | | VGQHTVEVLKSLGYDEEKINNFKDSKVVRY |
| 4 | HCL | Aquincola | MEEWNFPVEYDENYLPPADSRYWFPRRETMPAAERDKAILG |
| | | tertiaricarbonus | RLQQVCQYAWEHAPFYRRKWEEAGFQPSQLKSLEDFEARVP |
| | | L108 | VVKKTDLRESQAAHPPFGDYVCVPNSEIFHVHGTSGTTGRP |
| | | | TAFGIGRADWRAIANAHARIMWGMGIRPGDLVCVAAVFSLY |
| | | | MGSWGALAGAERLRAKAFPFGAGAPGMSARLVQWLDTMKPA |
| | | | AFYGTPSYAIHLAEVAREEKLNPRNFGLKCLFFSGEPGASV |
| | | | PGVKDRIEEAYGAKVYDCGSMAEMSPFMNVAGTEQSNDGML |
| | | | CWQDIIYTEVCDPANMRRVPYGQRGTPVYTHLERTSQPMIR |
| | | | LLSGDLTLWTNDENPCGRTYPRLPQGIFGRIDDMFTIRGEN |
| | | | IYPSEIDAALNQMSGYGGEHRIVITRESAMDELLLRVEPSE |
| | | | SVHAAGAAALETFRTEASHRVQTVLGVRAKVELVAPNSIAR |
| | | | TDFKARRVIDDREVFRALNQQLQSSAGSAWSHPQFEK |
| 5 | alkK | Pseudomonas | MLGQMMRNQLVIGSLVEHAARYHGAREVVSVETSGEVTRSC |
| | | oleovorans | WKEVELRARKLASALGKMGLTPSDRCATIAWNNIRHLEVYY |
| | | | AVSGAGMVCHTINPRLFIEQITYVINHAEDKVVLLDDTFLP |
| | | | IIAEIHGSLPKVKAFVLMAHNNSNASAQMPGLIAYEDLIGQ |
| | | | GDDNYIWPDVDENEASSLCYTSGTTGNPKGVLYSHRSTVLH |
| | | | SMTTAMPDTLNLSARDTILPVVPMFHVNAWGTPYSAAMVGA |
| | | | KLVLPGPALDGASLSKLIASEGVSIALGVPVVWQGLLAAQA |
| | | | GNGSKSQSLTRVVVGGSACPASMIREFNDIYGVEVIHAWGM |
| | | | TELSPFGTANTPLAHHVDLSPDEKLSLRKSQGRPPYGVELK |
| | | | IVNDEGIRLPEDGRSKGNLMARGHWVIKDYFHSDPGSTLSD |
| | | | GWFSTGDVATIDSDGFMTICDRAKDIIKSGGEWISTVELES |
| | | | IAIAHPHIVDAAVIAARHEKWDERPLLIAVKSPNSELTSGE |
| | | | VCNYFADKVARWQIPDAAIFVEELPRNGTGKILKNRLREKY |
| | | | GDILLRSSSSVCE |
| 6 | orfZ | Clostridium | MEWEEIYKEKLVTAEKAVSKIENHSRVVFAHAVGEPVDLVN |
| | | kluyveri | ALVKNKDNYIGLEIVHMVAMGKGEYTKEGMQRHFRHNALFV |
| | | | GGCTRDAVNSGRADYTPCFFYEVPSLFKEKRLPVDVALIQV |
| | | | SEPDKYGYCSFGVSNDYTKPAAESAKLVIAEVNKNMPRTLG |
| | | | DSFIHVSDIDYIVEASHPLLELQPPKLGDVEKAIGENCASL |
| | | | IEDGATLOLGIGAIPDAVLLFLKNKKNLGIHSEMISDGVME |
| | | | LVKAGVINNKKKTLHPGKIVVTFLMGTKKLYDFVNNNPMVE |
| | | | TYSVDYVNNPLVIMKNDNMVSINSCVQVDLMGQVCSESIGL |
| | | | KQISGVGGQVDFIRGANLSKGGKAIIAIPSTAGKGKVSRIT |
| | | | PLLDTGAAVTTSRNEVDYVVTEYGVAHLKGKTLRNRARALI |
| | | | NIAHPKFRESLMNEFKKRF |
| 7 | MatB | Rhizobium | MSNHLFDAMRAAAPGNAPFIRIDNTRTWTYDDAFALSGRIA |
| | T207S | trifolii | SAMDALGIRPGDRVAVQVEKSAEALILYLACLRSGAVYLPL |
| | | | NTAYTLAELDYFIGDAEPRLVVVASSARAGVETIAKPRGAI |
| | | | VETLDAAGSGSLLDLARDEPADFVDASRSADDLAAILYTSG |
| | | | TTGRSKGAMLTHGNLLSNALTLRDFWRVTAGDRLIHALPIF |
| | | | HSHGLFVATNVTLLAGASMFLLSKFDPEEILSLMPQATMLM |
| | | | GVPTFYVRLLQSPRLDKQAVANIRLFISGSAPLLAETHTEF |
| | | | QARTGHAILERYGMTETNMNTSNPYEGKRIAGTVGFPLPDV |
| | | | TVRVTDPATGLALPPEQTGMIEIKGPNVFKGYWRMPEKTAA |
| | | | EFTADGFFISGDLGKIDRDGYVHIVGRGKDLVISGGYNIYP |
| | | | KEVEGEIDQIEGVVESAVIGVPHPDFGEGVTAVVVRKPGAA |
| | | | LDEKAIVSALQDRLARYKQPKRIIFAEDLPRNTMGKVQKNI |
| | | | LRQQYADLYTRT |
| 8 | ACS | Arabidopsis | MASEENDLVFPSKEFSGQALVSSPQQYMEMHKRSMDDPAAF |
| | T324G | thaliana | WSDIASEFYWKQKWGDQVFSENLDVRKGPISIEWFKGGITN |
| | | | ICYNCLDKNVEAGLGDKTAIHWEGNELGVDASLTYSELLQR |
| | | | VCQLANYLKDNGVKKGDAVVIYLPMLMELPIAMLACARIGA |
| | | | VHSVVFAGFSADSLAQRIVDCKPNVILTCNAVKRGPKTINL |
| | | | KAIVDAALDQSSKDGVSVGICLTYDNSLATTRENTKWQNGR |
| | | | DVWWQDVISQYPTSCEVEWVDAEDPLFLLYTSGSTGKPKGV |
| | | | LHTTGGYMIYTATTFKYAFDYKSTDVYWCTADCGWIgGHSY |
| | | | VTYGPMLNGATVVVFEGAPNYPDPGRCWDIVDKYKVSIFYT |
| | | | APTLVRSLMRDDDKFVTRHSRKSLRVLGSVGEPINPSAWRW |
| | | | FFNVVGDSRCPISDTWWQTETGGFMITPLPGAWPQKPGSAT |
| | | | FPFFGVQPVIVDEKGNEIEGECSGYLCVKGSWPGAFRTLFG |
| | | | DHERYETTYFKPFAGYYFSGDGCSRDKDGYYWLTGRVDDVI |
| | | | NVSGHRIGTAEVESALVLHPQCAEAAVVGIEHEVKGQGIYA |
| | | | FVTLLEGVPYSEELRKSLVLMVRNQIGAFAAPDRIHWAPGL |
| | | | PKTRSGKIMRRILRKIASRQLEELGDTSTLADPSVVDQLIA |
| | | | LADV |
| 9 | ACS | Arabidopsis | MASEENDLVFPSKEFSGQALVSSPQQYMEMHKRSMDDPAAF |
| | W427S | thaliana | WSDIASEFYWKQKWGDQVFSENLDVRKGPISIEWFKGGITN |
| | | | ICYNCLDKNVEAGLGDKTAIHWEGNELGVDASLTYSELLQR |
| | | | VCQLANYLKDNGVKKGDAVVIYLPMLMELPIAMLACARIGA |
| | | | VHSVVFAGFSADSLAQRIVDCKPNVILTCNAVKRGPKTINL |
| | | | KAIVDAALDQSSKDGVSVGICLTYDNSLATTRENTKWQNGR |
| | | | DVWWQDVISQYPTSCEVEWVDAEDPLFLLYTSGSTGKPKGV |
| | | | LHTTGGYMIYTATTFKYAFDYKSTDVYWCTADCGWITGHSY |
| | | | VTYGPMLNGATVVVFEGAPNYPDPGRCWDIVDKYKVSIFYT |
| | | | APTLVRSLMRDDDKFVTRHSRKSLRVLGSVGEPINPSAWRW |
| | | | FFNVVGDSRCPISDTWsQTETGGFMITPLPGAWPQKPGSAT |
| | | | FPFFGVQPVIVDEKGNEIEGECSGYLCVKGSWPGAFRTLFG |
| | | | DHERYETTYFKPFAGYYFSGDGCSRDKDGYYWLTGRVDDVI |
| | | | NVSGHRIGTAEVESALVLHPQCAEAAVVGIEHEVKGQGIYA |
| | | | FVTLLEGVPYSEELRKSLVLMVRNQIGAFAAPDRIHWAPGL |
| | | | PKTRSGKIMRRILRKIASRQLEELGDTSTLADPSVVDQLIA |
| | | | LADV |
| 10 | ICS | Pseudomonas | MRDYEHVVESFDYLQSATQDLHGELTALNACVECCDRHAHG |
| | | chlororaphis | EAVALYCEAQDGHAERYRFRDLQRQAARFGNFLREQGVKPG |
| | | | DRVAGLMPRTVELLIAILGTWRIGAVYQPLFTAFGPKAIEQ |
| | | | RLNCSNARWIVTDPHNRPKLDDVTDCPSIVVTGGAPQNPAD |
| | | | HHFWSALNRQADDCAPVLLDASAPFLLMCTSGTTGPAKPLE |
| | | | VPLSAILAFKGYMRDAIDLRADDRFWNLADPGWAYGLYYAV |
| | | | TGPLACGYATLFYDGPFTVESTRHIIAKYAINNLAGSPTAY |
| | | | RFLIAAGAEFADAVRGRLRAVSSAGEPLNPQVVRWFAEQLG |
| | | | VVIHDHYGQTEIGMVLCNHHGLRHPVREGSAGYAVPGYRIV |
| | | | VLDKAHRELPAGQPGVLAVDRERSPLCWFDGYLGMPTQAFA |
| | | | GRYYLSGDIVELNDDGSISFVGRNDDLITTSGYRVGPFDVE |
| | | | SALIEHPAVVEAAVIGKPDPQRTELIKAFVVLNTPYLPSPE |
| | | | LAEELRLHVRQRLAAHAYPREMEFVDHLPKTPSGKLQRFIL |
| | | | RNQEIAKQQALG |
| 11 | matB | Streptomyces | MSSLFPALSPAPTGAPADRPALRFGERSLTYAELAAAAGAT |
| | | coelicolor | AGRIGGAGRVAVWATPAMETGVAVVAALLAGVAAVPLNPKS |
| | | | GDKELAHILSDSAPSLVLAPPDAELPPALGALERVDVDVRA |
| | | | RGAVPEDGADDGDPALVVYTSGTTGPPKGAVIPRRALATTL |
| | | | DALADAWQWTGEDVLVQGLPLFHVHGLVLGILGPLRRGGSV |
| | | | RHLGRFSTEGAARELNDGATMLFGVPTMYHRIAETLPADPE |
| | | | LAKALAGARLLVSGSAALPVHDHERIAAATGRRVIERYGMT |
| | | | ETLMNTSVRADGEPRAGTVGVPLPGVELRLVEEDGTPIAAL |
| | | | DGESVGEIQVRGPNLFTEYLNRPDATAAAFTEDGFFRTGDM |
| | | | AVRDPDGYVRIVGRKATDLIKSGGYKIGAGEIENALLEHPE |
| | | | VREAAVTGEPDPDLGERIVAWIVPADPAAPPALGTLADHVA |
| | | | ARLAPHKRPRVVRYLDAVPRNDMGKIMKRALNRD |
| 12 | ACS | Metallosphaera | MFMRYIMVEEQTLKTGSQELEEKADYNMRYYAHLMKLSKEK |
| | | sedula | PAEFWGSLAQDLLDWYEPWKETMRQEDPMTRWFIGGKINAS |
| | | | YNAVDRHLNGPRKFKAAVIWESELGERKIVTYQDMFYEVNR |
| | | | WANALRSLGVGKGDRVTIYMPLTPEGIAAMLASARIGAIHS |
| | | | VIFAGFGSQAIADRVEDAKAKVVITADAYPRRGKVVELKKT |
| | | | VDEALNSLGERSPVQHVLVYRRMKTDVNMKEGRDVFFDEVG |
| | | | KYRYVEPERMDSNDPLFILYTSGTTGKPKGIMHSTGGYLTG |
| | | | TAVMLLWSYGLSQENDVLFNTSDIGWIVGHSYITYSPLIMG |
| | | | RTVVIYESAPDYPYPDKWAEIIERYRATTFGTSATALRYFM |
| | | | KYGDEYVKNHDLSSIRIIVINGEVLNYSPWKWGLEVLGGGK |
| | | | VFMSHQWWQTETGAPNLGYLPGIIYMPMKSGPASGFPLPGN |
| | | | FVEVLDENGNPSAPRVRGYLVMRPPFPPNMMMGMWNDNGER |
| | | | LKKTYFSKFGSLYYPGDFAMVDEDGYIWVLGRADETLKIAA |
| | | | HRIGAGEVESAITSHPSVAEAAVIGVPDSVKGEEVHAFVVL |
| | | | KQGYAPSSELAKDIQSHVRKVMGPIVSPQIHFVDKLPKTRS |
| | | | GKVMRRVIKAVMMGSSAGDLTTIEDEASMDEIKKAVEELKK |
| | | | ELKTS |
| 13 | ACS | Sulfolobus | MTEKLSEQLQQLGEQNLEEKADYNMRYYKYLYKKSIEEPDK |
| | | tokodaii | FWGELAEELITWYEPWKQAFVQEEGLLTKWFVGGKLNASYN |
| | | | AVDRHLNSHRKYKAAIFWESEKGEKKVVTYQDLFYEVNKWA |
| | | | NALRELGVKKGDRVTIYMPLTPEGVIAKLAVARLGAIHSVV |
| | | | FAGFGAQALADRIADAGAKVVITADAYYRRGKLVELKKTVD |
| | | | EALNILGDKSPVQKVLVYKRTGTEIPFKEGRDVYFDEVGKY |
| | | | KYIEPVPVEATEPLFILYTSGTTGKPKGIVHSTGGYLVGTA |
| | | | VMLLWSYGLSQENDVLFNTSDIGWIVGHSYITYSPLVMGRS |
| | | | IVIYESAPDYPYPDKWAEMIEKYRATTFGTSATAIRTLMKY |
| | | | GEDYVKQHDLSSLRIIVINGEPLNYAPWKWGLEVVGGGKVF |
| | | | MSHQWWQTETGGPNIGYIPGVVYLPMKSGPAVGFALPGNKV |
| | | | TVVNEEGKETKPRERGYLVMLPPFPPMMMIGMWNDPDNERL |
| | | | KKTYFSKFPGIYYPGDYAMIDEDGYIWVMGRADETIKVAAH |
| | | | RIGAGEVESIVTSHPAVAEAAAVGIPDPVKGEAVHLFVVLK |
| | | | VGYKPSPQLAREIQEHVRKYMGAIVTPEVHFVDKLPKTRSG |
| | | | KIMRRVIKAVMMGQSAGDITTLEDEASMDEIKKAVEEFKKS |
| | | | LSQ |
| 14 | ACS | S. solfataricus | MVQEITENIKEIEEKVDYNTRIYREIYRESIENPGKFWGKL |
| | | | GEDLIDWFEPWKEIYKQETLTKWFLGGKLNASYNAIDRHLN |
| | | | SSRKFKAAIIWESEKGERKILTYQDLFYEVNRWANALKQLG |
| | | | VKKGDRVTIYMPLTPEGVTAMLACARIGAIHSVVFAGFGSQ |
| | | | ALADRIADAQSKIVITADGYYRKGRLIELKKTVDDALSKLQ |
| | | | DNSVKNIIIFRRIGIEIPFKEGRDVFFDEIGKYKYIEPEPV |
| | | | EATHPLFILYTSGTTGKPKGIVHSTGGYLVGTATMLLWSYG |
| | | | LSQENDVLFNTSDIGWIVGHSYITYSPLVMGRSVVIYESAP |
| | | | DYPTPDKWAELIEKYKATTFGTSATFLRYLMKYGEDYIKAH |
| | | | DLSSLRIIVTNGEPLNYAPWKYGLEIIGKGRVFMSHQWWQT |
| | | | ETGAPNLGYMPGYPIFLTMKSGPASGFPLPGNKIKVVDENG |
| | | | NPTRPRERGYLIIEPPFPPMMMIGMWNDDGNERVIKTYFSK |
| | | | FPNLYYTGDFAMIDEDGYVWVSGRADETLKIAGHRIGAGEV |
| | | | ESAITSHPAVAEAAVIGIPDPVKGEIAHAFVVLKQGYHQNN |
| | | | ELSKEIQEHVRKIMGPIVLLEVHFVNALPKTRSGKVMRRVI |
| | | | KAVMTGSNIGDISTLEDEASMEEIKKAIEVLRRQLNP |
| 15 | Nmar_1309 | Nitrosopumilus | MAAVKKIFDEIIETDHKVITEESSKSILKNYGVKVPPYALV |
| | | maritimus | TSAEEAAKEAKKIGFPLVMKVVSPQILHKTDVGGVKVGLDN |
| | | | VADVKKTFTDMYGRLSKKKGVNVKGILLEKMVPKGVELIVG |
| | | | IQNDSQFGPIIMVGMGGIMTEVMKDVAFRMLPITTSDAKSM |
| | | | LNELKGSKLLKGFRGSEPIDTNLVAKMLVNIGKLGVENADY |
| | | | INSIDFNPVIVYPKSHYVVDAKIILNKEKKKNSISKAKPSI |
| | | | TDMETFFTPKSVALVGASASPGKIGNSILDSLVNYDFKGKV |
| | | | YPINPKADKIFGQKCYPSVADIPGKVDLVVVSVDLSMTPPV |
| | | | LEDCAKKGVHSVVIVSGGGKELGGERAAYEAEVARLSKKHK |
| | | | IRIIGPNCIGMFNAANRLDCAFQGQERMVRSKLGPVAFFSQ |
| | | | SGTMGISMLESADTFGLSKMISFGNRSDVDEADMIWYAAND |
| | | | PQTKVIGLYVEGFGDGRKFINVAKRVMKEKKKPIVIWKSGR |
| | | | TAAGAKQAASHTGSLGGSNAIIMGAFKQAGIISVDSYQELA |
| | | | GVLKALAWQPAAKGNKVAMTSNGAGPMIGGIDQLEKFGLAI |
| | | | GKLSPKLLKKMKSRFPPAVPIHNGNPADVGGGATADDYQFV |
| | | | IQQFMDEKNIDIAMPWFVFQDDPLEETIVDHLAGFQKKAKK |
| | | | PLLCGGNGGPYTEKMIKLIEKHNVPVYQDLRTWVAAASALH |
| | | | QWGKISKK |
| 16 | | | |
| 17 | | | |
| 18 | | | |
| 19 | phaC | Cupriavidus | MATGKGAAASTQEGKSQPFKVTPGPFDPATWLEWSRQWQGT |
| | | necator | EGNGHAAASGIPGLDALAGVKIAPAQLGDIQQRYMKDFSAL |
| | | | WQAMAEGKAEATGPLHDRRFAGDAWRTNLPYRFAAAFYLLN |
| | | | ARALTELADAVEADAKTRQRIRFAISQWVDAMSPANFLATN |
| | | | PEAQRLLIESGGESLRAGVRNMMEDLTRGKISQTDESAFEV |
| | | | GRNVAVTEGAVVFENEYFQLLQYKPLTDKVHARPLLMVPPC |
| | | | INKYYILDLQPESSLVRHVVEQGHTVFLVSWRNPDASMAGS |
| | | | TWDDYIEHAAIRAIEVARDISGQDKINVLGFCVGGTIVSTA |
| | | | LAVLAARGEHPAASVTLLTTLLDFADTGILDVFVDEGHVQL |
| | | | REATLGGGAGAPCALLRGLELANTFSFLRPNDLVWNYVVDN |
| | | | YLKGNTPVPFDLLFWNGDATNLPGPWYCWYLRHTYLQNELK |
| | | | VPGKLTVCGVPVDLASIDVPTYIYGSREDHIVPWTAAYAST |
| | | | ALLANKLRFVLGASGHIAGVINPPAKNKRSHWTNDALPESP |
| | | | QQWLAGAIEHHGSWWPDWTAWLAGQAGAKRAAPANYGNARY |
| | | | RAIEPAPGRYVKAKA |
| 20 | phaC | Chromobacterium | MQQFVNSLSLGQDQSDAPHPLTGAWSQLMSQTNQLLQLQSS |
| | | USM2 | LYQQQLGLWTQFLGQTAGNDASAPSAKPSDRRFASPEWDEH |
| | | | PFYSFLKQSYLQTSKWMMELVDKTQIDESAKDKLSFATRQY |
| | | | LDAMAPSNFMLTNPDVVKRAIETQGESLVEGMKNMMEDIQK |
| | | | GHISMSDESKFQIGKNLVVTPGEVVFRNELIELIQYTPTTE |
| | | | KVHEKPLLFVPPCINKYYLMDLQPDNSMVRHFVGQGYRVFL |
| | | | VSWRSAVPEMKNFTWETYIEKGVFAAAEAVQKITKQPTMNA |
| | | | LGFCVGGVILTTALCVAQAKGLKYFDSATFMTSLIDHAEPG |
| | | | EISFFIDEALVASREAKMAAGGIISGKEIGRTFASLRANDL |
| | | | VWNYVVNNYLLGKTPAPFDLLYWNNDAVDLPLPMHTFMLRQ |
| | | | FYINNALITPGAITLCGVPIDISKIDIPVYMFAAREDHIVL |
| | | | WSSAYSGLKYLSGTPSRRFVLGASGHIAGSINPVTKDKRNY |
| | | | WTNEQLPVNPEEWLEGAQSHPGSWWKDWDAWLAPQSGKQVP |
| | | | APKMLGSKEFPPLQPAPGSYVLAKAMPPVAAALN |
| 21 | phaC1437 | Pseudomonas sp. | MSNKSNDELKYQASENTLGLNPVVGLRGKDLLASARMVLRQ |
| | | MBEL 6-19 | AIKQPVHSVKHVAHFGLELKNVLLGKSGLQPTSDDRRFADP |
| | | | AWSQNPLYKRYLQTYLAWRKELHDWIDESNLAPKDVARGHF |
| | | | VINLMTDAMAPTNTAANPAAVKRFFETGGKSLLDGLSHLAK |
| | | | DLVHNGGMPSQVNMGAFEVGKSLGVTEGAVVFRNDVLELIQ |
| | | | YKPTTEQVYERPLLVVPPQINKFYVFDLSPDKSLARFCLRN |
| | | | NVQTFIVSWRNPTKEQREWGLSTYIEALKEAVDVVTAITGS |
| | | | KDVNMLGACSGGITCTALLGHYAAIGENKVNALTLLVTVLD |
| | | | TTLDSDVALFVNEQTLEAAKRHSYQAGVLEGRDMAKVFAWM |
| | | | RPNDLIWNYWVNNYLLGNEPPVFDILFWNNDTTRLPAAFHG |
| | | | DLVELFKNNPLIRPNALEVCGTPIDLKQVTADIFSLAGTND |
| | | | HITPWKSCYKSAQLFGGNVEFVLSSGGHIKSILNPPGNPKS |
| | | | RYMTSTEVAENADEWQANATKHTDSWWLHWQAWQAQRSGEL |
| | | | KKSPTKLGSKAYPAGEAAPGTYVHER |
| 22 | phaC | Allochromatium | MFPIDIRPDKLTQEMLDYSRKLGQGMENLLNAEAIDTGVSP |
| | | vinosum | KQAVYSEDKLVLYRYDRPEGAPEAQPVPLLIVYALVNRPYM |
| | | | TDIQEDRSTIKGLLATGQDVYLIDWGYPDQADRALTLDDYI |
| | | | NGYIDRCVDYLREAHGVDKVNLLGICQGGAFSLMYSALHPD |
| | | | KVRNLVTMVTPVDFKTPDNLLSAWVQNVDIDLAVDTMGNIP |
| | | | GELLNWTFLSLKPFSLTGQKYVNMVDLLDDPDKVKNFLRME |
| | | | KWIFDSPDQAGETFRQFIKDFYQNNGFLNGGVVLGGQEVDL |
| | | | KDITCPVLNIFALQDHLVPPDASRALKGLTSSPDYTELAFP |
| | | | GGHIGIYVSGKAQKEVTPAIGKWLNER |
| 23 | phaE | Allochromatium | MSNTNFFNDDWLELQRKYWDNWTDMSRKAMGLDSASSSATT |
| | | vinosum | PWEAAIDQWWKAMAPAAPDLSRSFMEKMMEQGKNFFRLADT |
| | | | FAKRADEGNAGNGLELWTKTLEDMQKRFSGSLDDGGNTMQR |
| | | | LMSFWELPLDNWQRMMSSMSPMPGDMLRNMPHEQFKDSLDR |
| | | | ALSAPGLGYTREEQSQYQELMRSAMEYQAALQEYTNVYTKL |
| | | | GMKSVEHMGSYIQGVIDSGKTIDSARALYDNWVACCEGAYA |
| | | | DEVATPEYARIHGRLVNAQMALKKRMSILVDENLGALNMPT |
| | | | RSELRTLQDRLQETRRENKALRHSLHSLERRVAALAGEEPA |
| | | | TKPATALRSPAPAAKAPARRRTTKTNPAD |
| 24 | phaC | Thiocapsa | MSPFPIDIRPDKLTEEMLEYSRKLGEGMQNLLKADQIDTGV |
| | | pfennigi | TPKDVVHREDKLVLYRYRRPAQVATQTIPLLIVYALVNRPY |
| | | | MTDIQEDRSTIKGLLATGQDVYLIDWGYPDQADRALTLDDY |
| | | | INGYIDRCVDYLRETHGVDQVNLLGICQGGAFSLCYTALHS |
| | | | EKVKNLVTMVTPVDFQTPGNLLSAWVQNVDVDLAVDTMGNI |
| | | | PGELLNWTFLSLKPFSLTGQKYVNMVDLLDDEDKVKNFLRM |
| | | | EKWIFDSPDQAGETFRQFIKDFYQRNGFINGGVLIGDQEVD |
| | | | LRNIRCPVLNIYPMQDHLVPPDASKALAGLTSSEDYTELAF |
| | | | PGGHIGIYVSGKAQEGVTPAIGRWLNERG |
| 25 | phaE | Thiocapsa | MNDTANKTSDWLDIQRKYWETWSELGRKTLGLEKTPANPWA |
| | | pfennigi | GALDHWWQTVSPAAPNDLVRDFMEKLAEQGKAFFGLTDYFT |
| | | | KGLGGSSGTQGWDTLSKTIDDMQKAFASGRIEGDETFRRLM |
| | | | AFWEMPLDNWQRTMSSLSPVPGDLLRNMPHDQVRDSVDRIL |
| | | | SAPGLGYTREEQARYQDLIRRSLEYQSALNEYNGFFGQLGV |
| | | | KSLERMRAFLQGQAEKGVAIESARTLYDAWVGCCEEVYAEE |
| | | | VSSADYAHIHGRLVNAQMALKQRMSTMVDEVLGAMPLPTRS |
| | | | ELRTLQDRLQESRGEGKRQRQEIETLKRQVAALAGGAQPAP |
| | | | QASAQPSTRPAPATAPAASAAPKRSTTTRRKTTKPTTGQ |
| 26 | phaC | Bacillus | MAIPYVQEWEKLIKSMPSEYKSSARRFKRAYEIMTAEAEPE |
| | | megaterium | VGLTPKEVIWKKNKAKLYRYTPVKDNLHKTPILLVYALINK |
| | | | PYILDLTPGNSLVEYLLNRGFDVYLLDWGTPGLEDSNMKLD |
| | | | DYIVDYIPKAAKKVLRTSKSPDLSVLGYCMGGTMTSIFAAL |
| | | | NEDLPIKNLIFMTSPFDFSDTGLYGAFLDDRYFNLDKAVDT |
| | | | FGNIPPEMIDFGNKMLKPITNFYGPYVTLVDRSENQRFVES |
| | | | WKLMQKWVADGIPFAGEAYRQWIRDFYQQNKLINGELEVRG |
| | | | RKVDLKNIKANILNIAASRDHIAMPHQVAALMDAVSSEDKE |
| | | | YKLLQTGHVSVVFGPKAVKETYPSIGDWLEKRSK |
| 27 | phaE | Bacillus | MEQQKVFDPFQAWKDVYDKTESYWGKVIGDNMNREEFSQLM |
| | | megaterium | GNVLNMNLQYQQAVNEVTGRYLHQVNVPTKEDVANVASLVI |
| | | | NVEEKVELLEEQFDDRFGELEAQQESASALKKDVTKLKSDV |
| | | | KSLDKKLDKVLSLLEGQQKTQDELKETIQKQIKTQGEQLQA |
| | | | QLLEKQEKLAEKPKAEAKSEAKPSNAQKTEQPARK |
| 28 | phaE | Candidatus | MENKNPNEQLGGVVNAWADMQKRMWGDWSSLLQNLPGGSEG |
| | | Accumulibacter | PVEAAKKGVAAASKGTNEAARMLMDRMTSSQGAMNRVMDFF |
| | | phophatis | FKSMKIVAPNLEANKDWRPDLKGFAEQWAKESTAMLERSFG |
| | | clade IIA | MGSHLGNLSSTLSKDLPDAMGPWLSFLMQAASSGHVGEAML |
| | | str. UW-1 | GGTSGINRLLSMEGDAALAGVGEIPLFGASREKNAKLLRLV |
| | | | DAVVDLRKNSLTFHTAFGDALAKAVEATVEELGKVAAKGEK |
| | | | ITAVRQLMSLWYRTADKSLLVTFNTQEFLDKQNAFTAAQQQ |
| | | | FKLAQRAVVEDIFRGLDMPTRSELDETYQVIHELKKEVRAL |
| | | | KKALLPAAPAAVAKSSPAPRKAAAARAKSE |
| 29 | phaC | Candidatus | MFSFPIQILPADVAAETAALNEKLAKGISNLTNLTDDDIDI |
| | | Accumulibacter | GSTPKDVVFEQDGIKVYHYHALAEPSQIMKTPLLIVPPLIN |
| | | phophatis | GYEVADLQPDRSLVRNLLNQGIDVYLNDWGYPRQVDKYRTL |
| | | clade IIA | DDYINGYFDDTVDFIRKHHGVDKIALFGICQGGAMSTTYST |
| | | str. UW-1 | LNPDKISHLVLTVSPIDFDAYKANHKPHEGLMFTMGADADV |
| | | | EKMVAVHGNVPATVLNESFMMASPFILNYGKYADVIDILDD |
| | | | RLALQNFLRMEKWLFGGPDAGGQMFKEFIRDFLKGNKLVKG |
| | | | TLEIGGRKVDLKELTIPILNIFAEKDHIVPPPCTVALGKHV |
| | | | GSKDYTEFAIATGHIGIYTGGLSQKVLAPTVGKWMRERGA |
| 30 | phaC | Aeromonas | MSQPSYGPLFEALAHYNDKLLAMAKAQTERTAQALLQTNLD |
| | | caviae | DLGQVLEQGSQQPWQLIQAQMNWWQDQLKLMQHTLLKSAGQ |
| | | | PSEPVITPERSDRRFKAEAWSEQPIYDYLKQSYLLTARHLL |
| | | | ASVDALEGVPQKSRERLRFFTRQYVSAMAPSNFLATNPELL |
| | | | KLTLESGGQNLVRGLALLAEDLERSADQLNIRLTDESAFEL |
| | | | GRDLALTPGRVVQRTELYELIQYSPTTETVGKTPVLIVPPF |
| | | | INKYYIMDMRPQNSLVAWLVAQGQTVFMISWRNPGVAQAQI |
| | | | DLDDYVVDGVIAALDGVEAATGEREVHGIGYCIGGTALSLA |
| | | | MGWLAARRQKQRVRTATLFTTLLDFSQPGELGIFIHEPIIA |
| | | | ALEAQNEAKGIMDGRQLAVSFSLLRENSLYWNYYIDSYLKG |
| | | | QSPVAFDLLHWNSDSTNVAGKTHNSLLRRLYLENQLVKGEL |
| | | | KIRNTRIDLGKVKTPVLLVSAVDDHIALWQGTWQGMKLFGG |
| | | | EQRFLLAESGHIAGIINPPAANKYGFWHNGAEAESPESWLA |
| | | | GATHQGGSWWPEMMGFIQNRDEGSEPVPARVPEEGLAPAPG |
| | | | HYVKVRLNPVFACPTEEDAA |
| 31 | phaC | Casimicrobium | MSTKKPGEAGNNPFAAWMENNPFAAAAANNPFAAGSADNPF |
| | | | AGGGKAGANPFAAGLDAMQTMMSGNLGQQPWAASAMPSFAG |
| | | | APAAPEAGWWWLPAVKPEAFQAAAMKWFSPQTDVAALAAAD |
| | | | RRFRGEGWQSQPFFQAVRDQYLRTAAFWRDVVEHADLDDRE |
| | | | RHRAKFFLEQFLDASAPSNFFLTNPEAIQRAVETKGESVKH |
| | | | GMENLAHDIEAGHIAMTDETAFKVGENLAVTPGQVVFRNEL |
| | | | IEIIHYTPTQKTVYQRPLLIVPPCINKFYILDLKPENSFVA |
| | | | HAVAQGFNVYLVSWRNVPEELKTLTWEDYLEEGALTAIDEV |
| | | | RSHAGIEKINVLGFCVGGTILASALGVLAARGELDDFIESA |
| | | | TYLTTLLDFSEPGDIKAYLGESTYQMRAQQFGPDGTGGMMK |
| | | | GSELAQSFASLRANDLIWNYVVNNYLKGQDPPAFDLLFWNG |
| | | | DSTNLPGPMYLYYIRNFYLDNKLTEHGGINMLGEDVDLALV |
| | | | DVPTFVYCSREDHIVPWKSAFASAELWGGDVEFVMGASGHI |
| | | | AGVINPPGPKKRSYWTGNFPAPTPEAWDSKAKEHPGSWWPH |
| | | | WYTWLAPHSGKRVAAPKQPKSKLGAAPGTYVLAKA |
| 32 | phaC | Burkholderiales | MSSKKSSTKGDAAGAQRAENPFAAWASGNPFLSGVANPFAN |
| | | | AAQDATAKFAEMMNAAPGANPFAEGIQAMQTLMSSNIGQQP |
| | | | SIVSTVPAFAQPGPMPMPEAGWWWLPSVQPTVWQQALTTWF |
| | | | SPQTDVAALAAADRRFRSKSWESQPFFQLARDHYLRNCAFW |
| | | | REVVSKADLDDRERHRARFFVEQVLDATAPTNFFLTNPEAI |
| | | | ERAIETKGESVKHGIENLSHDIEAGHIAMTDEKAFKVGENL |
| | | | AVTPGQVVFRNELIELIHYTPTEKTVYQRPLLIVPPCINKF |
| | | | YILDLKPENSFVAHAVAQGFDVYLVSWRNVGDDLKALTWED |
| | | | YLEEGVLTAIDETRDHSGAATINTLGFCVGGTILSCALAVL |
| | | | ASRGELDDFVESATYLTTLLDFTEPGDIKAYLGESTYQMRV |
| | | | QQFGPDGAGGMMKGSELAQSFASLRANDLIWNYVVNNYLKG |
| | | | QDPPAFDLLYWNGDSTNLPGPMYLYYLRNFYLDNKLTKPGT |
| | | | LDMIGEPVDLSNVDIPTYVYCSREDHIVPWKSAFASAELWG |
| | | | GDVEFVVGASGHIAGVINPPGPKKRSYWTGRWPADTPEAWD |
| | | | AKASEHAGSWWPHWYAWLAPHSGKRVPAKKQGKSPLGAAPG |
| | | | QFVLEKA |
| 33 | phaC | Rhizobacter | MSTESQIPPSLQSLMSSAWGAAAAPSFPGMPTMPGMPTLPI |
| | | | GPGMPTMAAPEAGWWWLPAVNPGAMQAAATRWFSPETDVSA |
| | | | LAASDRRFRAPSWQAQPYFQATRDQYLRTAAYWRELVAHAD |
| | | | LEEKERHRAKFFLEQMLDAVAPSNFFLTNPEAIERAIETKG |
| | | | ESLRHGIENLKHDIETGHIAMTDETVFQVGGNLALTPGQVV |
| | | | FRNDLIELLHYTPTQKTLHSRPLLIVPPCINKFYILDLKPE |
| | | | NSFVGHAVAQGFDVYLVSWRNIPEALRALTWEDYLEQGVLT |
| | | | AIDEVREHAGVDTINTLGFCVGGTILSCALAVLAARGELDD |
| | | | FVESATYLTTLLDFSEPGDIKAYLGESTFKLREQQLNKDGD |
| | | | TGLMKGSELAQSFASLRANDLIWNYVVNNYLKGQDPPAFDL |
| | | | LYWNGDSTNLPGPMYLYYMRNFYLDNKLMEPDALTMLDEPI |
| | | | DLSSVNIATYVYCSREDHIVPWKSAYASAELWGGEVEFVLG |
| | | | ASGHIAGVVNPPGPKKRSYWTGRWPAASTDEWDAKAKETPG |
| | | | SWWPHWYAWLAERSGKKVVKKSPSKSKLGAAPGSYVLEKM |
| 34 | phaC | Betaproteo- | MDFSDYTKAHQAWQEFLSKNAGAMGGMGAGAAGMPSFAFPN |
| | | bacteria | AQFAFPSGAGSMPQLEAGWWWLPSVKPEAWQAAAKWYALDT |
| | | | KLDDLIAKDRRFRSEAWTKQPFFQGIRDQYVRTAAFWRDLL |
| | | | QGAALDDKEKQRARFFLEQWLDAIAPTNFFATNPEAIEKAI |
| | | | ETKGESLKHGIENLVHDIERGHIAMTDESAFAVGKNVALSK |
| | | | GEVIFRNDLIELIHYAPTQKTVHERPLLIVPPCINKFYILD |
| | | | LKPENSFVGHAVAAGFDVYLVSWRNIPEALKTLVWEDYLES |
| | | | GVLTAIDEVREHASVETINTLGFCVGGTILACSLAVLAARG |
| | | | ELEEFVESATYLTTLLDFSEPGDIQAYLGESTYAMRVKQLG |
| | | | EDGTNGLMSGAELAQAFASLRANDLIWSFVVKNYLKGEDPP |
| | | | AFDLLYWNSDSTNLPGPMYLYYLRNFYLDNKLTQPGALTML |
| | | | EEPIDLSLVTIDSYVYSSREDHIVPWKSAFASAELWGGDVE |
| | | | FVLGASGHIAGVVNPPIPVKRSYWTSAWPAENPEAWDAKSK |
| | | | EHPGSWWPHWYTWLAKRSGKKSAPRKHKVSKLGAAPGQYVL |
| | | | EKI |
| 35 | phaC | Blastococcus | MSSPSPDPAKWLRDLMQTEPAALWPAVNIADTGKQVAAVAA |
| | | | PWTKAVADFTAMQLTAVQQMTAPWTAALPGLGAAAEPVKDK |
| | | | RFAGDEWTKDPRYEAVVRTYLTQSDLLHKALDAAPLDEHSK |
| | | | AQWGFALRQVIDALSPANTLATNPEAMQLAMETGGASLVDG |
| | | | LQLFTEDLAKGRVSMTDEMAFEVGRDVGTTPGGVVYQNDLM |
| | | | QLIQYTPTTAKVHKRPLVIVPPCINKFYILDLRPTNSFVAH |
| | | | AVAQGHTVLLVSWRNAGPAQDRLTWDDYLEQGVLKAIDVAR |
| | | | SITKADKVNTLGFCVGGTLLASALAVQAARGEQPAASMTLL |
| | | | TTMLDFTDTGEIGVLVTEPAVVAREAAIGRGGLLKGSELGQ |
| | | | VFASLRANDLIWPYVVKGYLQGQAPPAFDMLFWNGDETNLP |
| | | | GPMFCWYVRNAYLENKLREPGGTVQLGQPVDLAAVDVPAFV |
| | | | YASKEDHIVPWQTAYASTQILSGDTTFVLGASGHIAGVINP |
| | | | PAANKRNYWTRPDPEGTDGAEGPVPLDADPDRWFEAAERVP |
| | | | GSWWPAWAAWLTPHAGPQVAARKKLGNAEFAVLEEAPGSYV |
| | | | REPAS |
| 36 | phaC | Burkholderia | MFESWLNAWRGFADPARAATASAAVNPFATFQFPASFPFQM |
| | | cepacia | PSMPDLGGLAGLGGMASPFAGLKLPVAAIPPERLQALQADY |
| | | | ARDCMTLMQQAAAAKLESPELKDRRFSGDAWKSSPAHAFAA |
| | | | AWYLLNARYLQELADSLQTDPKTRERIRFTVQQWTAAAAPS |
| | | | NFLALNPDAQKSILDTQGESLRQGMMNLLGDLQRGKISQTD |
| | | | ESQFVVGKNLGCTEGAVVYENDLIPADPSTRRRRTRCFERP |
| | | | LLIVSAVHQQVLHPRPAAREFASSRTRCRTVIRCFLVSWRN |
| | | | ADASVAHKTWDDYMNEGLLAAIDAVQQVSGREQINTLGFCV |
| | | | GGTMLATALAVLAARGEHPAASMTLLTAMLDFSDTGILDVF |
| | | | VDEAHVQMREQTIGGKNGTQPGLMRGVEFANTFSFLRPNDL |
| | | | VWNYVVDNYLKGRTPAPFDLLYWNSDSTSLPGPMYAWYLRN |
| | | | TYLENKLREPGALTVCGESVDLSLIDVPTFIYGSREDHIVP |
| | | | WQTAYASTSILSGPLKFVLGASGHIAGVINPPAKKKRSYWV |
| | | | NDDLPESADDWFAGATEQPGSWWPTWVEWLDAYGGRKVAPP |
| | | | AEAGSAQFPVIEPAPGRYVLQRD |
| 37 | phaC | Rhodococcus | MLDHVHKKLKSTLDPIGWGPAVTSVAGRAVRNPQAVTAATA |
| | (phbC) | ruber | EYTGRLAKIPAAATRVFNANDPDAPMPVDPRDRRFSDTAWQ |
| | | | ENPAYFSLLQSYLATRAYVEELTEAGSGDPLQDGKARQFAN |
| | | | LMFDALAPSNFLWNPGVLTRAFETGGASLLRGARYAAHDIL |
| | | | NRGGLPLKVDSDAFTVGENLAATPGKVVFRNDLIELIQYAP |
| | | | QTEQVHAVPILAAPPWINKYYILDLAPGRSLAEWAVQHGRT |
| | | | VFMISYRNPDESMRHITMDDYYVDGIATALDVVEEITGSPK |
| | | | IEVLSICLGGAMAAMAAARAFAVGDKRVSAFTMLNTLLDYS |
| | | | QVGELGLLTDPATLDLVEFRMRQQGFLSGKEMAGSFDMIRA |
| | | | KDLVFNYWVSRWMKGEKPAAFDILAWNEDSTSMPAEMHSHY |
| | | | LRSLYGRNELAEGLYVLDGQPLNLHDIACDTYVVGAINDHI |
| | | | VPWTSSYQAVNLLGGDVRYVLTNGGHVAGAVNPPGKRVWFK |
| | | | AVGAPDAESGTPLPADPQVWDEAATRYEHSWWEDWTAWSNK |
| | | | RAGELVAPPAMGSTAHPPLEDAPGTYVFS |
| 38 | phaC | Rhodococcus | MSDTPLPTIPDELTAPLDLLLTSGSRSVAARMLPDSSWTRL |
| | | opacus 1 | GVGLAGRPGTVARRGGALVRELGAIAAGTSDRTPAKSDKRF |
| | | | GDAAWQQNPALRRAMQAYLATSHTAAALLDDAELDWRDHER |
| | | | MRFVLDNLVEGLSPTNNPLLSPLGWKAMVDTGGLSAARGVR |
| | | | ALVRDMLSKPRVPAMVEPDAFAVGEDVAATKGAVVLQTRTF |
| | | | ELIHYTPQTEKVHATPLLIVPPVINKYYILDIAPGRSLIEY |
| | | | LLQQGQQVFAISWRNPHARHRDWDADTYGSAIVEALDTVQC |
| | | | VAGTDSAHVLGTCSGGILAAMVAAHLTEIGEGDRIAGLTLA |
| | | | VTVLDQTQAGTAAAVMSERAAAAAIRDSAARGYLDGRTLAE |
| | | | MFAWLRPSDLVWRYWVNNYVQGRAPAAFDVLFWNSDTTRMT |
| | | | AALHRDLVLLGLRNALTAPGAATMLGTPVDLSTVAADAYVV |
| | | | GGSADHLCPWQSTYRSARLLGSKDSRFVLSSNGHIASLVNP |
| | | | PGNPRASFRFGQPVPETPDEWLAAAETASDSWWPDYARWLA |
| | | | ERSGPDVDAPHGLGARQFPPLAPAPGTYVHHS |
| 39 | phaC | Rhodococcus | MLDIAWNPTLNTLTRNAWRLSFGGGIAPVQRTPSTRIHEAP |
| | | opacus 2 | HQDLYRFDSTETDGGRPVLLVPPLAAPAHCFDLRPGQSLAA |
| | | | HLVGSGKAAYLVDYGTMGYSDRGLGFEDWIDDFIPTAVDRV |
| | | | SRLHDGAPVDLIGWSLGGTMSLLTAAGRNYLPIGSVTAIGT |
| | | | PIDYESVTMIAPLRMVGRFTGDRPLTTATRAMGGLPAPLVQ |
| | | | ASYRFTALQRELTKPWFIARNLHDTETLARMESIDRFMADM |
| | | | PGYPARFFRQVCTELILGNALAAGSFQTRNGAIALADLAVP |
| | | | VLAIGGTEDVIAPIPSVRAATDVLTGSPSVRFESAPGSHLG |
| | | | LVAGPKAKDSTWAHIDGFLDGVVAQTA |
| 40 | phaC | Rhodococcus | MSPIGPVVDIARGLVREVERTQLRARRGIELIARRPPPQVS |
| | | opacus 3 | NTPKDEVWSFGKAKLWRYRNDDVRHGPPVLMFLGLVGDSAI |
| | | | FDLFPGNSWAEKLVAEGFDVFLFDWGRPEAAEGEHDLGTYM |
| | | | DGYFVPAVDAVRRIAGADEVSVGAYCMGSLMLTLLLGSRSN |
| | | | VPVRNAVLFAPPCDYDHAPGFLTGFKDGRLETRHVVDEMTG |
| | | | LVPEDAVRGMFRLLQPTSDIVQYVTLWENLWRDGYADAHRA |
| | | | INHWAWDHRSMAAPSFVEMVEDYVRENRLVNGGAELAGRPV |
| | | | DLHSITIPLLMIIAEKDEFVPPANSEPLAELVGSDDVEILR |
| | | | IPGGHAGALMGSAARKKTMPGVVDWLRRHSDTPPR |
| 41 | | | |
| 42 | | | |
| 43 | | | |
| 44 | MMO | uncultured | DEVRHISNGYATLLMALADEGNHQLLARDLRYAWWNNHRVV |
| | | bacterium | DAAIGTFIEYGTRDRRKDRESYAEMWRRWIYDDYYRSYLVP |
| | | | LEKYGLEIPHDLIEEAWNQIWNKGYVHEVAQFFATGWLANY |
| | | | WRIDPMTDKDFEWFENK |
| 45 | mmoB | Methylococcus | MSVNSNAYDAGIMGLKGKDFADQFFADENQVVHESDTVVLV |
| | | capsulatus | LKKSDEINTFIEEILLTDYKKNVNPTVNVEDRAGYWWIKAN |
| | | (Bath) | GKIEVDCDEISELLGRQFNVYDFLVDVSSTIGRAYTLGNKF |
| | | | TITSELMGLDRKLEDYHA |
| 46 | mmoC | Methylococcus | MQRVHTITAVTEDGESLRFECRSDEDVITAALRQNIFLMSS |
| | | capsulatus | CREGGCATCKALCSEGDYDLKGCSVQALPPEEEEEGLVLLC |
| | | (Bath) | RTYPKTDLEIELPYTHCRISFGEVGSFEAEVVGLNWVSSNT |
| | | | VQFLLQKRPDECGNRGVKFEPGQFMDLTIPGTDVSRSYSPA |
| | | | NLPNPEGRLEFLIRVLPEGRFSDYLRNDARVGQVLSVKGPL |
| | | | GVFGLKERGMAPRYFVAGGTGLAPVVSMVRQMQEWTAPNET |
| | | | RIYFGVNTEPELFYIDELKSLERSMRNLTVKACVWHPSGDW |
| | | | EGEQGSPIDALREDLESSDANPDIYLCGPPGMIDAACELVR |
| | | | SRGIPGEQVFFEKFLPSGAA |
| 47 | momoD | Methylococcus | MVESAFQPFSGDADEWFEEPRPQAGFFPSADWHLLKRDETY |
| | | capsulatus | AAYAKDLDFMWRWVIVREERIVQEGCSISLESSIRAVTHVL |
| | | (Bath) | NYFGMTEQRAPAEDRTGGVQH |
| 48 | mmoX | Methylococcus | MALSTATKAATDALAANRAPTSVNAQEVHRWLQSFNWDFKN |
| | | capsulatus | NRTKYATKYKMANETKEQFKLIAKEYARMEAVKDERQFGSL |
| | | (Bath) | QDALTRLNAGVRVHPKWNETMKVVSNFLEVGEYNAIAATGM |
| | | | LWDSAQAAEQKNGYLAQVLDEIRHTHQCAYVNYYFAKNGQD |
| | | | PAGHNDARRTRTIGPLWKGMKRVFSDGFISGDAVECSLNLQ |
| | | | LVGEACFTNPLIVAVTEWAAANGDEITPTVFLSIETDELRH |
| | | | MANGYQTVVSIANDPASAKYLNTDLNNAFWTQQKYFTPVLG |
| | | | MLFEYGSKFKVEPWVKTWNRWVYEDWGGIWIGRLGKYGVES |
| | | | PRSLKDAKQDAYWAHHDLYLLAYALWPTGFFRLALPDQEEM |
| | | | EWFEANYPGWYDHYGKIYEEWRARGCEDPSSGFIPLMWFIE |
| | | | NNHPIYIDRVSQVPFCPSLAKGASTLRVHEYNGQMHTFSDQ |
| | | | WGERMWLAEPERYECQNIFEQYEGRELSEVIAELHGLRSDG |
| | | | KTLIAQPHVRGDKLWTLDDIKRLNCVFKNPVKAFN |
| 49 | mmoY | Methylococcus | MSMLGERRRGLTDPEMAAVILKALPEAPLDGNNKMGYFVTP |
| | | capsulatus | RWKRLTEYEALTVYAQPNADWIAGGLDWGDWTQKFHGGRPS |
| | | (Bath) | WGNETTELRTVDWFKHRDPLRRWHAPYVKDKAEEWRYTDRF |
| | | | LQGYSADGQIRAMNPTWRDEFINRYWGAFLFNEYGLFNAHS |
| | | | QGAREALSDVTRVSLAFWGFDKIDIAQMIQLERGFLAKIVP |
| | | | GFDESTAVPKAEWTNGEVYKSARLAVEGLWQEVFDWNESAF |
| | | | SVHAVYDALFGQFVRREFFQRLAPRFGDNLTPFFINQAQTY |
| | | | FQIAKQGVQDLYYNCLGDDPEFSDYNRTVMRNWTGKWLEPT |
| | | | IAALRDFMGLFAKLPAGTTDKEEITASLYRVVDDWIEDYAS |
| | | | RIDFKADRDQIVKAVLAGLK |
| 50 | mmoZ | Methylococcus | MAKLGIHSNDTRDAWVNKIAQLNTLEKAAEMLKQFRMDHTT |
| | | capsulatus | PFRNSYELDNDYLWIEAKLEEKVAVLKARAFNEVDFRHKTA |
| | | (Bath) | FGEDAKSVLDGTVAKMNAAKDKWEAEKIHIGFRQAYKPPIM |
| | | | PVNYFLDGERQLGTRLMELRNLNYYDTPLEELRKQRGVRVV |
| | | | HLQSPH |
| 51 | | | |
| 52 | | | |
| 53 | | | |
| 54 | ADH | Corynebacterium | MTTAAPQEFTAAVVEKFGHDVTVKDIDLPKPGPHQALVKVL |
| | | glutamicum | TSGICHTDLHALEGDWPVKPEPPFVPGHEGVGEVVELGPGE |
| | | | HDVKVGDIVGNAWLWSACGTCEYCITGRETQCNEAEYGGYT |
| | | | QNGSFGQYMLVDTRYAARIPDGVDYLEAAPILCAGVTVYKA |
| | | | LKVSETRPGQFMVISGVGGLGHIAVQYAAAMGMRVIAVDIA |
| | | | DDKLELARKHGAEFTVNARNEDSGEAVQKYTNGGAHGVLVT |
| | | | AVHEAAFGQALDMARRAGTIVFNGLPPGEFPASVFNIVFKG |
| | | | LTIRGSLVGTRQDLAEALDFFARGLIKPTVSECSLDEVNGV |
| | | | LDRMRNGKIDGRVAIRF |
| 55 | ACDH | Corynebacterium | MTVYANPGTEGSIVNYEKRYENYIGGKWVPPVEGQYLENIS |
| | | glutamicum | PVTGEVFCEVARGTAADVELALDAAHAAADAWGKTSVAERA |
| | | | LILHRIADRMEEHLEEIAVAETWENGKAVRETLAADIPLAI |
| | | | DHFRYFAGAIRAQEDRSSQIDHNTVAYHFNEPIGVVGQIIP |
| | | | WNFPILMATWKLAPALAAGNAIVMKPAEQTPASILYLINII |
| | | | GDLIPEGVLNIVNGLGGEAGAALSGSNRIGKIAFTGSTEVG |
| | | | KLINRAASDKIIPVTLELGGKSPSIFFSDVLSQDDAFAEKA |
| | | | VEGFAMFALNQGEVCTCPSRALVHESIADEFLELGVKRVQN |
| | | | IKLGNPLDTETMMGAQASQEQMDKISSYLKIGPEEGAQTLT |
| | | | GGKVNKVDGMENGYYIEPTVFRGTNDMRIFREEIFGPVLSV |
| | | | ATFSDFDEAIRIANDTNYGLGAGVWSRDQNTIYRAGRAIQA |
| | | | GRVWVNQYHNYPAHSAFGGYKESGIGRENHLMMLNHYQQTK |
| | | | NLLVSYDPNPTGLF |
| 56 | adhe | E. Coli | MAVTNVAELNALVERVKKAQREYASFTQEQVDKIFRAAALA |
| | | | AADARIPLAKMAVAESGMGIVEDKVIKNHFASEYIYNAYKD |
| | | | EKTCGVLSEDDTFGTITIAEPIGIICGIVPTTNPTSTAIFK |
| | | | SLISLKTRNAIIFSPHPRAKDATNKAADIVLQAAIAAGAPK |
| | | | DLIGWIDQPSVELSNALMHHPDINLILATGGPGMVKAAYSS |
| | | | GKPAIGVGAGNTPVVIDETADIKRAVASVLMSKTFDNGVIC |
| | | | ASEQSVVVVDSVYDAVRERFATHGGYLLQGKELKAVQDVIL |
| | | | KNGALNAAIVGQPAYKIAELAGFSVPENTKILIGEVTVVDE |
| | | | SEPFAHEKLSPTLAMYRAKDFEDAVEKAEKLVAMGGIGHTS |
| | | | CLYTDQDNQPARVSYFGQKMKTARILINTPASQGGIGDLYN |
| | | | FKLAPSLTLGCGSWGGNSISENVGPKHLINKKTVAKRAENM |
| | | | LWHKLPKSIYFRRGSLPIALDEVITDGHKRALIVTDRFLFN |
| | | | NGYADQITSVLKAAGVETEVFFEVEADPTLSIVRKGAELAN |
| | | | SFKPDVIIALGGGSPMDAAKIMWVMYEHPETHFEELALRFM |
| | | | DIRKRIYKFPKMGVKAKMIAVTTTSGTGSEVTPFAVVTDDA |
| | | | TGQKYPLADYALTPDMAIVDANLVMDMPKSLCAFGGLDAVT |
| | | | HAMEAYVSVLASEFSDGQALQALKLLKEYLPASYHEGSKNP |
| | | | VARERVHSAATIAGIAFANAFLGVCHSMAHKLGSQFHIPHG |
| | | | LANALLICNVIRYNANDNPTKQTAFSQYDRPQARRRYAEIA |
| | | | DHLGLSAPGDRTAAKIEKLLAWLETLKAELGIPKSIREAGV |
| | | | QEADFLANVDKLSEDAFDDQCTGANPRYPLISELKQILLDT |
| | | | YYGRDYVEGETAAKKEAAPAKAEKKAKKSA |
| 57 | Sbm | Escherichia | MSNVQEWQQLANKELSRREKTVDSLVHQTAEGIAIKPLYTE |
| | | coli | ADLDNLEVTGTLPGLPPYVRGPRATMYTAQPWTIRQYAGFS |
| | | | TAKESNAFYRRNLAAGQKGLSVAFDLATHRGYDSDNPRVAG |
| | | | DVGKAGVAIDTVEDMKVLFDQIPLDKMSVSMTMNGAVLPVL |
| | | | AFYIVAAEEQGVTPDKLTGTIQNDILKEYLCRNTYIYPPKP |
| | | | SMRIIADIIAWCSGNMPRENTISISOYHMGEAGANCVQQVA |
| | | | FTLADGIEYIKAAISAGLKIDDFAPRLSFFFGIGMDLFMNV |
| | | | AMLRAARYLWSEAVSGFGAQDPKSLALRTHCQTSGWSLTEQ |
| | | | DPYNNVIRTTIEALAATLGGTQSLHINAFDEALGLPTDFSA |
| | | | RIARNTQUIQEESELCRTVDPLAGSYYIESLTDQIVKQARA |
| | | | DIQQIDEAGGMAKAIEAGLPKRMIEEASAREQSLIDQGKRV |
| | | | IVGVNKYKLDHEDETDVLEIDNVMVRNEQIASLERIRATRD |
| | | | DAAVTAALNALTHAAQHNENLLAAAVNAARVRATLGEISDA |
| | | | LEVAFDRYLVPSQCVTGVIAQSYHQSEKSASEFDAIVAQTE |
| | | | QFLADNGRRPRILIAKMGQDGHDRGAKVIASAYSDLGFDVD |
| | | | LSPMFSTPEEIARLAVENDVHVVGASSLAAGHKTLIPELVE |
| | | | ALKKWGREDICVVAGGVIPPQDYAFLQERGVAAIYGPGTPM |
| | | | LDSVRDVLNLISQHHD |
| 58 | mcr | Chloroflexus | MSGTGRLAGKIALITGGAGNIGSELTRRFLAEGATVIISGR |
| | | aurantiacus | NRAKLTALAERMQAEAGVPAKRIDLEVMDGSDPVAVRAGIE |
| | | | AIVARHGQIDILVNNAGSAGAQRRLAEIPLTEAELGPGAEE |
| | | | TLHASIANLLGMGWHLMRIAAPHMPVGSAVINVSTIFSRAE |
| | | | YYGRIPYVTPKAALNALSQLAARELGARGIRVNTIFPGPIE |
| | | | SDRIRTVFQRMDQLKGRPEGDTAHHFLNTMRLCRANDQGAL |
| | | | ERRFPSVGDVADAAVFLASAESAALSGETIEVTHGMELPAC |
| | | | SETSLLARTDLRTIDASGRTTLICAGDQIEEVMALTGMLRT |
| | | | CGSEVIIGFRSAAALAQFEQAVNESRRLAGADFTPPIALPL |
| | | | DPRDPATIDAVFDWGAGENTGGIHAAVILPATSHEPAPCVI |
| | | | EVDDERVLNFLADEITGTIVIASRLARYWQSQRLTPGARAR |
| | | | GPRVIFLSNGADQNGNVYGRIQSAAIGQLIRVWRHEAELDY |
| | | | QRASAAGDHVLPPVWANQIVRFANRSLEGLEFACAWTAQLL |
| | | | HSQRHINEITLNIPANISATTGARSASVGWAESLIGLHLGK |
| | | | VALITGGSAGIGGQIGRLLALSGARVMLAARDRHKLEQMQA |
| | | | MIQSELAEVGYTDVEDRVHIAPGCDVSSEAQLADLVERTLS |
| | | | AFGTVDYLINNAGIAGVEEMVIDMPVEGWRHTLFANLISNY |
| | | | SLMRKLAPLMKKQGSGYILNVSSYFGGEKDAAIPYPNRADY |
| | | | AVSKAGQRAMAEVFARFLGPEIQINAIAPGPVEGDRLRGTG |
| | | | ERPGLFARRARLILENKRLNELHAALIAAARTDERSMHELV |
| | | | ELLLPNDVAALEQNPAAPTALRELARRFRSEGDPAASSSSA |
| | | | LLNRSIAAKLLARLHNGGYVLPADIFANLPNPPDPFFTRAQ |
| | | | IDREARKVRDGIMGMLYLQRMPTEFDVAMATVYYLADRNVS |
| | | | GETFHPSGGLRYERTPTGGELFGLPSPERLAELVGSTVYLI |
| | | | GEHLTEHLNLLARAYLERYGARQVVMIVETETGAETMRRLL |
| | | | HDHVEAGRLMTIVAGDQIEAAIDQAITRYGRPGPVVCTPFR |
| | | | PLPTVPLVGRKDSDWSTVLSEAEFAELCEHQLTHHFRVARK |
| | | | IALSDGASLALVTPETTATSTTEQFALANFIKTTLHAFTAT |
| | | | IGVESERTAQRILINQVDLTRRARAEEPRDPHERQQELERF |
| | | | IEAVLLVTAPLPPEADTRYAGRIHRGRAITV |
| 59 | | | |
| 60 | | | |
| 61 | | | |
| 62 | groEL | Escherichia | MAAKDVKFGNDARVKMLRGVNVLADAVKVTLGPKGRNVVLD |
| | | coli | KSFGAPTITKDGVSVAREIELEDKFENMGAQMVKEVASKAN |
| | | | DAAGDGTTTATVLAQAIITEGLKAVAAGMNPMDLKRGIDKA |
| | | | VTAAVEELKALSVPCSDSKAIAQVGTISANSDETVGKLIAE |
| | | | AMDKVGKEGVITVEDGTGLQDELDVVEGMQFDRGYLSPYFI |
| | | | NKPETGAVELESPFILLADKKISNIREMLPVLEAVAKAGKP |
| | | | LLIIAEDVEGEALATLVVNTMRGIVKVAAVKAPGFGDRRKA |
| | | | MLQDIATLTGGTVISEEIGMELEKATLEDLGQAKRVVINKD |
| | | | TTTIIDGVGEEAAIQGRVAQIRQQIEEATSDYDREKLQERV |
| | | | AKLAGGVAVIKVGAATEVEMKEKKARVEDALHATRAAVEEG |
| | | | VVAGGGVALIRVASKLADLRGQNEDQNVGIKVALRAMEAPL |
| | | | RQIVLNCGEEPSVVANTVKGGDGNYGYNAATEEYGNMIDMG |
| | | | ILDPTKVTRSALQYAASVAGLMITTECMVTDLPKNDAADLG |
| | | | AAGGMGGMGGMGGMM |
| 63 | groES | Escherichia | MNIRPLHDRVIVKRKEVETKSAGGIVLTGSAAAKSTRGEVL |
| | | coli | AVGNGRILENGEVKPLDVKVGDIVIFNDGYGVKSEKIDNEE |
| | | | VLIMSESDILAIVEA |
| 64 | groEL2 | Methylococcus | MAKEVVYRGSARQRMMQGIEILARAAIPTLGATGPSVMIQH |
| | | capsulatus | RADGLPPISTRDGVTVANSIVLKDRVANLGARLLRDVAGTM |
| | | | SREAGDGTTTAIVLARHIAREMFKSLAVGADPIALKRGIDR |
| | | | AVARVSEDIGARAWRGDKESVILGVAAVATKGEPGVGRLLL |
| | | | EALDAVGVHGAVSIELGORREDLLDVVDGYRWEKGYLSPYF |
| | | | VTDRARELAELEDVYLLMTDREVVDFIDLVPLLEAVTEAGG |
| | | | SLLIAADRVHEKALAGLLLNHVRGVFKAVAVTAPGFGDKRP |
| | | | NRLLDLAALTGGRAVLEAQGDRLDRVTLADLGRVRRAVVSA |
| | | | DDTALLGIPGTEASRARLEGLRLEAEQYRALKPGQGSATGR |
| | | | LHELEEIEARIVGLSGKSAVYRVGGVTDVEMKERMVRIENA |
| | | | YRSVVSALEEGVLPGGGVGFLGSMPVLAELEARDADEARGI |
| | | | GIVRSALTEPLRIIGENSGLSGEAVVAKVMDHANPGWGYDQ |
| | | | ESGSFCDLHARGIWDAAKVLRLALEKAASVAGTFLTTEAVV |
| | | | LEIPDTDAFAGFSAEWAAATREDPRV |
| 65 | groES1 | Methylococcus | MKIRPLHDRVIIKRLEEERTSAGGIVIPDSAAEKPMRGEIL |
| | | capsulatus | AVGNGKVLDNGEVRALQVKVGDKVLFGKYAGTEVKVDGEDV |
| | | | VVMREDDILAVLES |
| 66 | groES2 | Methylococcus | MKIRPLHDRVVVIRREEEKTSPGGIVIPDTAKEKPIKGEIV |
| | | capsulatus | AVGTGKVLDNGQVRPLAVKAGDTVLFGKYSGTEIKIDGTEY |
| | | | LMLREDDIMGVIES |

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that various changes and modifications may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated. Note that elements recited in means-plus-function format are intended to be construed in accordance with 35 U.S.C. § 112 ¶6. The scope of the invention is therefore defined by the following claims.

All references cited herein are incorporated by reference as if each had been individually incorporated by reference in its entirety. In describing embodiments of the present application, specific terminology is employed for the sake of clarity. However, the invention is not intended to be limited to the specific terminology so selected. Nothing in this specification should be considered as limiting the scope of the present invention.

All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. An engineered microorganism, comprising a CoA-ligase and a PHA polymerase, capable of producing a poly(HIBA) from a feedstock.

2. The engineered microorganism of claim 1, wherein the poly(HIBA) comprises poly(2-HIBA) and/or poly(3-HIBA).

3. The engineered microorganism of claim 1, wherein the CoA-ligase has at least 90% sequence identity to one or more of isocaprenoyl-CoA:2-hydroxyisocaproate CoA-transferase (HadA) from Clostridium difficile (SEQ ID NO: 3), isobutyrate-CoA synthetase (ICS) from Pseudomonas chlororaphis (SEQ ID NO: 10), NMar_1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12).

4. The engineered microorganism of claim 1, wherein the PHA synthase has at least 90% sequence identity to PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

5. The engineered microorganism of any of the preceding claims, wherein the engineered microorganism further comprises an engineered pathway for producing a hydroxyisobutyric acid (HIBA) from the feedstock.

6. The engineered microorganism of claim 5, wherein the feedstock comprises methane, ethane, propane, methanol, ethanol, propanol, glycerol, glucose, succinic acid and combinations thereof.

7. The engineered microorganism of claim 5, wherein the HIBA comprises 2-hydroxyisobutyric acid (2-HIBA) and/or 3-hydroxyisobutyric acid (3-HIBA).

8. The engineered microorganism of claim 5, wherein the engineered pathway comprises or consists of MMO, ADH, ACDH, and/or acetyl-CoA synthase.

9. The engineered microorganism of claim 5, wherein the engineered pathway further comprises or consists of a sleeping beauty mutase (Sbm).

10. The engineered microorganism of claim 9, wherein the engineered pathway further comprises or consists of a methylmalonyl-CoA reductase (mmcr).

11. The engineered microorganism of claim 1, wherein the engineered microorganism is Escherichia coli.

12. A method of producing a poly(hydroxyisobutyric acid) (poly(HIBA)) from a feedstock, the method comprising:
1) providing a nutrient medium comprising the feedstock; and
2) culturing an engineered microorganism in the nutrient medium, the engineered microorganism comprising a CoA-ligase and a polyhydroxyalkanoate (PHA) polymerase.

13. The method of claim 12, wherein the poly(HIBA) comprises or consists of poly(2-hydroxyisobutyric acid) (poly(2-HIBA)) and/or poly(3-hydroxyisobutyric acid) (poly (3-HIBA)).

14. The method of claim 12, wherein the CoA-ligase comprises or consists of one or more of Isocaprenoyl-CoA:2-hydroxyisocaproate CoA-transferase (HadA) from Clostridium difficile (SEQ ID NO: 3), isobutyrate-CoA synthetase from Pseudomonas chlororaphis (SEQ ID NO: 10), NMar_1309 from Nitrosopumilus maritimus SCM1 (SEQ ID NO: 15), HCL from A. tertiaricarbonus L108 (SEQ ID NO: 4), acs from Sulfolobus solfataricus (SEQ ID NO: 14), and/or 3HP-CoA synthetase from Metallosphaera sedula (SEQ ID NO: 12).

15. The method of claim 12, wherein the PHA synthase comprises one or more of PhaC-PhaE from Allochromatium vinosum (SEQ ID NO: 22 and 23), phaC1 from Chromobacterium USM2 (SEQ ID NO: 20), PhaC1437 from Pseudomonas (SEQ ID NO: 21), PHA polymerase 3 from Rhodococcus opacus PD630 (SEQ ID NO: 40), and/or phaC from Betaproteobacterium (SEQ ID NO: 34).

16. The method of claim 12, wherein the engineered microorganism further comprises or consists of an engineered pathway for producing a hydroxyisobutyric acid (HIBA) from the feedstock.

17. The method of claim 16, wherein the feedstock comprises or consists of methane, ethane, propane, methanol, ethanol, propanol, and combinations thereof.

18. The method of claim 16, wherein the HIBA comprises or consists of 2-hydroxyisobutyric acid (2-HIBA) and/or 3-hydroxyisobutyric acid (3-HIBA).

19. The method of claim 16, wherein the engineered pathway comprises or consists of MMO, ADH, ACDH, and/or acetyl-CoA synthase.

20. The method of claim 19, wherein the engineered pathway further comprises or consists of a sleeping beauty mutase (Sbm).

21. The method of claim 20, wherein the engineered pathway further comprises or consists of a methylmalonyl-CoA reductase (mmcr).

22. The method of any of claims 12-21, further comprising (i) separating the microorganism from the nutrient medium; (ii) optionally extracting the poly(HIBA) from the microorganism; and (iii) heating the poly(HIBA) to a temperature in a range from about 150° C. to about 450° C. for a time period from about 0.5 to 120 minutes to produce methacrylic acid (MAA).

23. The method of claim 22, further comprising esterifying the MAA with an alcohol to produce a methacrylate ester (MAE).

24. The method of claim 21, further comprising separating the poly(HIBA) from the nutrient medium; depolymerize the poly(HIBA) to the HIBA; and converting the HIBA using a catalyst to produce a methacrylic acid (MAA).
